(19) United States
(12) Patent Application Publication
(10) Pub. No.: US 2014/0087416 A1
(43) Pub. Date: Mar. 27, 2014

(54) METHODS AND COMPOSITIONS FOR PRODUCING INDUCED HEPATOCYTES

(71) Applicant: Genentech, Inc., South San Francisco, CA (US)

(72) Inventors: Kamen P. Simeonov, San Francisco, CA (US); Hirdesh Uppal, San Ramon, CA (US)

(73) Assignee: Genentech, Inc., South San Francisco, CA (US)

(21) Appl. No.: 14/019,677

(22) Filed: Sep. 6, 2013

Related U.S. Application Data
(60) Provisional application No. 61/698,359, filed on Sep. 7, 2012.
(60) Provisional application No. 61/777,973, filed on Mar. 12, 2013.

(51) Int. Cl. C12N 5/071

(52) U.S. Cl. CPC C12N 5/067
USPC 435/32; 435/377; 435/455; 435/29; 536/23.5

(57) ABSTRACT

The present invention relates to methods and compositions for use in generating induced hepatocytes by reprogramming non-hepatocyte cells.

### RELATED APPLICATIONS

This application claims the benefit of U.S. Provisional Application No. 61/777,973, filed on 12 Mar. 2013, and U.S. Provisional Application No. 61/698,359, filed on 7 Sep. 2012, both of which are incorporated by reference herein in their entireties.

### SEQUENCE LISTING

The instant application contains a Sequence Listing which has been submitted in ASCII format via EFS-Web and is hereby incorporated by reference in its entirety. Said ASCII copy, created on Aug. 15, 2013, is named P4968R1-US_SL.txt and is 47,522 bytes in size.

### FIELD OF THE INVENTION

The present invention relates to methods and compositions for use in producing induced hepatocytes by reprogramming non-hepatocyte cells.

### BACKGROUND OF THE INVENTION

Takahashi and Yamanaka reported in 2006 that introduction of genes encoding four protein factors (Oct3/4, Sox2, c-Myc, and Klf4) into differentiated mouse adult fibroblasts induced these cells to become pluripotent stem cells (induced pluripotent stem cells). (See Takahashi and Yamanaka (2006) Cell 126:663-676.) Following this original report, pluripotent stem cells were also induced in human cells by transforming human somatic cells with genes encoding similar human protein factors (OCT4, SOX2, KLF4, and c-MYC), or by transforming human somatic cells with genes encoding human OCT4 and SOX2 factors plus genes encoding two other human factors (NANOG and LIN28). (See Takahashi et al., (2007) Cell 131:861-872 and Yu et al., (2007) Science 318:1917-1920, respectively; see Huangfu et al., (2008) Nature Biotechnology 26:1269-1275.) These reported methods used retroviruses or lentiviruses to integrate genes encoding the reprogramming factors into the genomes of the transformed cells. The genomes of the resulting reprogrammed cells, however, contained viral DNA, which could result in deleterious genetic consequences. A number of subsequent studies addressed this issue by using non-integrating adenovirus and lentivirus (Sommer 2009 Stem Cells 27:543-549); transient expression vectors (Okita 2008 Science 322; 949-953); and targeted integration and excision of vector sequences (Kaji 2009 Nature 458:771-775; Woltjen 2009 Nature 458:766-770). These approaches, however, still involve the use of viruses, genetic integration, or DNA plasmid vectors, and therefore, present a variety of biological and regulatory obstacles for clinical applications. Several studies have addressed this by describing virus-free and DNA-free pluripotency reprogramming methods. Pluripotency reprogramming factors have been introduced as recombinant proteins, synthetic mRNAs, and synthetic miRNAs. (See, e.g., Zhou et al., (2009) Cell Stem Cell 4:381-384; Warren et al., (2010) Cell Stem Cell 7:618-630; and Miyoshi et al., (2011) Cell Stem Cell 8:633-638.)

Two recent reports described the direct conversion of mouse fibroblasts to hepatocyte-like cells, without first passing through a pluripotent intermediate state, by introducing into mouse fibroblasts combinations of various genes encoding defined transcription factors. (See Huang et al., (2011) Nature 475:386-389; and Sekiya and Suzuki (2011) Nature 475:390-393.) In these reports, Huang identified the combination of Gata4, Hnf1α, and Foxa3 (along with inactivation of p19^{Arf}) as being sufficient to induce murine hepatic conversion; Sekiya and Suzuki identified three specific combinations of two transcription factors, comprising Hnf4α plus Foxa1, Foxa2, or Foxa3, that induced murine hepatic conversion. As in the very earliest reprogramming studies mentioned above, both Huang et al. and Sekiya and Suzuki used lentivirus- or retrovirus-mediated transduction to express transcription factors in mouse fibroblasts. However, while reprogramming to pluripotency has been demonstrated using a variety of non-integrating and non-DNA-based methods, as described in the studies referenced above, a direct conversion of one somatic cell to another has never been described without the use of integrating lentiviral or retroviral-mediated delivery of reprogramming factors, using neither mouse nor human cells.

While direct conversions of fibroblasts have been achieved in mouse cells, including neurons, hematopoietic cells, cardiomyocytes, and hepatocytes, only a few of these have been successfully applied to human cells. (See Vierbuchen et al., (2010) Nature 463:1035-1041; Szabo et al., (2010) Nature 468:521-526; Ieda et al., (2010) 142:375-386; Huang et al., (2011) Nature 475:386-389; Sekiya & Suzuk (2011) Nature 475:390-393; Pang et al., (2011) Nature 476:220-223; and Ieda et al., (2010) Cell 142:375-386.) While recent reports described success at identifying factors and processes necessary and sufficient for reprogramming mouse fibroblasts to mouse hepatocyte-like cells, the same methodologies may not necessarily be successful when applied to human cells. Scientific literature has indicated that factors and processes necessary and sufficient for altering the differentiation state of mouse cells may be distinct from that necessary and sufficient for altering the differentiation state of human cells. (See, e.g., Yi et al., (2012) Cell Research 22:616-619; Gonzalez et al., (2011) Nature Reviews Genetics 12:231-242; Pang et al., (2011) Nature 476:220-223; Vierbuchen & Wernig (2011) Nature Biotechnology 29:892-907.)

The generation of human induced hepatocytes from non-hepatocyte cells, without any risk of genomic alteration, offers great promise as a means for treating disease through cell transplantation. Human induced hepatocytes generated from human non-hepatocyte cells obtained from individual patients may enable development of patient-specific therapies which do not have a risk of immune rejection, thus eliminating the need for immunosuppressive procedures. Additionally, generation of human induced hepatocytes from disease-specific non-hepatocyte cells could provide a means to model and study specific disease states and develop therapeutics useful for the treatment of these diseases.

Therefore, there exists a need in the art to identify the key transcription factors necessary and sufficient to reprogram human non-hepatocyte cells into human induced hepatocytes. The present invention meets this need by providing methods and compositions useful for generating human induced hepatocytes from human non-hepatocyte cells.

### SUMMARY OF THE INVENTION

The present invention provides, in part, methods and compositions for reprogramming non-hepatocyte cells to induced hepatocytes, populations of induced hepatocytes, compositions comprising induced hepatocytes, and uses thereof. In some embodiments, the non-hepatocyte cells are non-hepatocyte somatic cells. In other embodiments, the compositions and methods provided herein are useful for producing human induced hepatocytes by reprogramming human non-hepatocyte cells.

The methods and compositions provided herein are useful for reprogramming a starting cell or starting cell population (e.g., a non-hepatocyte cell or non-hepatocyte cell population) to an induced hepatocyte or a population of induced hepatocytes with high efficiency. In some embodiments, the starting cell or starting cell population is a human non-hepatocyte cell or a human non-hepatocyte cell population (e.g, a non-hepatocyte cell of human origin), and the resulting induced hepatocytes are human induced hepatocytes or a population of human induced hepatocytes.

The present inventors have identified FOXA1, FOXA2, FOXA3, HNF1A, HNF4A, and GATA4 (and various combinations thereof) as key reprogramming factors that can reprogram human non-hepatocyte cells to human induced hepatocytes. The present inventors have also identified CEBPA, GATA6, HHEX, HNF1B, HNF6A, FOXA1, FOXA2, FOXA3, GATA4, HNF1A, and HNF4A as key reprogramming factors that can reprogram human non-hepatocyte cells to human induced hepatocytes.

In some embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, contacting the non-hepatocyte with an agent which increases or induces the expression or activity of one or more reprogramming factors, and culturing the non-hepatocyte cell under conditions suitable for reprogramming a non-hepatocyte cell, thereby producing an induced hepatocyte from the non-hepatocyte cell. In some embodiments, the agent increases or induces the expression or activity of one or more of the following factors: FOXA1, FOXA2, FOXA3, HNF1A, HNF4A, and GATA4. In other embodiments, the agent is a nucleic acid encoding one or more of the following factors: FOXA1, FOXA2, FOXA3, HNF1A, HNF4A, and GATA4. In other embodiments, the agent is one or more of the following factors: FOXA1, FOXA2, FOXA3, HNF1A, HNF4A, and GATA4. In yet other embodiments, the agent increases or induces the expression or activity of one or more of the following factors: CEBPA, GATA6, HHEX, HNF1B, HNF6A, FOXA1, FOXA2, FOXA3, GATA4, HNF1A, and HNF4A. In yet other embodiments, the agent is a nucleic acid encoding one or more of the following factors: CEBPA, GATA6, HHEX, HNF1B, HNF6A, FOXA1, FOXA2, FOXA3, GATA4, HNF1A, and HNF4A. In further embodiments, the agent is one or more of the following factors: CEBPA, GATA6, HHEX, HNF1B, HNF6A, FOXA1, FOXA2, FOXA3, GATA4, HNF1A, and HNF4A. In some embodiments, the nucleic acid is a nucleic acid capable of expressing a reprogramming factor. In some embodiments, the nucleic acid is RNA or mRNA.

In some embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding one or more reprogramming factors, and culturing the non-hepatocyte cell under conditions suitable for reprogramming a non-hepatocyte cell to an induced hepatocyte, thereby producing an induced hepatocyte from the non-hepatocyte cell. In some embodiments, the nucleic acid preparation is a mixture of nucleic acids (e.g., a nucleic acid preparation comprising a mixture of different nucleic acid molecules) encoding one or more reprograming factors.

In some embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules capable of expressing one or more reprogramming factors, and culturing the non-hepatocyte cell under conditions suitable for reprogramming a non-hepatocyte cell to an induced hepatocyte, thereby producing an induced hepatocyte from the non-hepatocyte cell. In some embodiments, the nucleic acid preparation is a mixture of nucleic acids (e.g., a nucleic acid preparation comprising a mixture of different nucleic acid molecules) encoding one or more reprograming factors and capable of expressing said one or more reprogramming factors.

In some embodiments, the nucleic acid preparation useful for reprogramming non-hepatocyte cells to induced hepatocytes comprises nucleic acid molecules encoding FOXA1, FOXA2, FOXA3, HNF1A, HNF4A, and GATA4. In some embodiments, the nucleic acid preparation useful for reprogramming non-hepatocyte cells to induced hepatocytes comprises nucleic acid molecules encoding CEBPA, GATA6, HHEX, HNF1B, HNF6A, FOXA1, FOXA2, FOXA3, GATA4, HNF1A, and HNF4A. In some embodiments, the nucleic acid preparation useful for reprogramming non-hepatocyte cells to induced hepatocytes comprises nucleic acid molecules encoding FOXA1, FOXA2, FOXA3, HNF1A, and HNF4A. In some embodiments, the nucleic acid preparation useful for reprogramming non-hepatocyte cells to induced hepatocytes comprises nucleic acid molecules encoding FOXA1, FOXA3, HNF1A, and HNF4A, nucleic acid molecules encoding FOXA1, FOXA2, HNF1A, and HNF4A, or nucleic acid molecules encoding FOXA2, FOXA3, HNF1A, and HNF4A. In some embodiments, the nucleic acid preparation useful for reprogramming non-hepatocyte cells to induced hepatocytes comprises nucleic acid molecules encoding FOXA1, HNF1A, and HNF4A. In some embodiments, the nucleic acid preparation useful for reprogramming non-hepatocyte cells to induced hepatocytes comprises nucleic acid molecules encoding FOXA3, HNF1A, and HNF4A. In some embodiments, the nucleic acid preparation useful for reprogramming non-hepatocyte cells to induced hepatocytes comprises nucleic acid molecules encoding FOXA2, HNF1A, and HNF4A. In some embodiments, the nucleic acid preparation useful for reprogramming non-hepatocyte cells to induced hepatocytes comprises nucleic acid molecules encoding FOXA1, FOXA3, and HNF1A, nucleic acid molecules encoding FOXA1, FOXA2, and HNF1A, or nucleic acid molecules encoding FOXA2, FOXA3, and HNF1A. In certain embodiments, the nucleic acid molecules encoding the reprogramming factors are RNA molecules or mRNA molecules.

In some embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, a nucleic acid sequence comprising SEQ ID NO:41, and a nucleic acid sequence comprising SEQ ID NO:43, and culturing the non-hepatocyte cell under conditions suitable for reprogramming, thereby producing an induced hepatocyte from the non-hepatocyte cell. In some embodiments, the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41. In some embodiments, the nucleic acid preparation comprises nucleic acid molecules comprising a nucleic acid sequence comprising SEQ ID NO:33, a nucleic acid sequence comprising SEQ ID NO:37, a nucleic acid sequence comprising SEQ ID NO:39, and a nucleic acid sequence comprising SEQ ID NO:41. In some embodiments, the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:39, and a nucleic acid sequence comprising SEQ ID NO:41. In some embodiments, the nucleic acid preparation comprises nucleic acid molecules comprising a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41. In some embodiments, the nucleic acid preparation comprises nucleic acid molecules comprising a nucleic acid sequence comprising SEQ ID NO:33, a nucleic acid sequence comprising SEQ ID NO:39, and a nucleic acid sequence comprising SEQ ID NO:41. In some embodiments, the nucleic acid preparation comprises nucleic acid molecules comprising a nucleic acid sequence comprising SEQ ID NO:37, a nucleic acid sequence comprising SEQ ID NO:39, and a nucleic acid sequence comprising SEQ ID NO:41. In some embodiments, the nucleic acid preparation comprises nucleic acid molecules comprising a nucleic acid sequence comprising SEQ ID NO:35, a nucleic acid sequence comprising SEQ ID NO:39, and a nucleic acid sequence comprising SEQ ID NO:41. In some embodiments, the nucleic acid preparation comprises nucleic acid molecules comprising a nucleic acid sequence comprising SEQ ID NO:33, a nucleic acid sequence comprising SEQ ID NO:37, and a nucleic acid sequence comprising SEQ ID NO:39. In some embodiments, the nucleic acid preparation comprises nucleic acid molecules comprising a nucleic acid sequence comprising SEQ ID NO:33, a nucleic acid sequence comprising SEQ ID NO:35, and a nucleic acid sequence comprising SEQ ID NO:39. In some embodiments, the nucleic acid preparation comprises nucleic acid molecules comprising a nucleic acid sequence comprising SEQ ID NO:35, a nucleic acid sequence comprising SEQ ID NO:37, and a nucleic acid sequence comprising SEQ ID NO:39.

In some embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:36, an amino acid sequence comprising SEQ ID NO:38, an amino acid sequence comprising SEQ ID NO:40, an amino acid sequence comprising SEQ ID NO:42, and an amino acid sequence comprising SEQ ID NO:44, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte. In some embodiments, the nucleic acid preparation comprises nucleic acid molecules encoding polypeptides encoding an amino acid sequence comprising SEQ ID NO:34, an amino acid sequence comprising SEQ ID NO:36, an amino acid sequence comprising SEQ ID NO:38, an amino acid sequence comprising SEQ ID NO:40, and an amino acid sequence comprising SEQ ID NO:42. In some embodiments, the nucleic acid preparation comprises nucleic acid molecules encoding polypeptides encoding an amino acid sequence comprising SEQ ID NO:34, an amino acid sequence comprising SEQ ID NO:38, an amino acid sequence comprising SEQ ID NO:40, and an amino acid sequence comprising SEQ ID NO:42. In some embodiments, the nucleic acid preparation comprises nucleic acid molecules encoding polypeptides encoding an amino acid sequence comprising SEQ ID NO:34, an amino acid sequence comprising SEQ ID NO:36, an amino acid sequence comprising SEQ ID NO:40, and an amino acid sequence comprising SEQ ID NO:42. In some embodiments, the nucleic acid preparation comprises nucleic acid molecules encoding polypeptides encoding an amino acid sequence comprising SEQ ID NO:36, an amino acid sequence comprising SEQ ID NO:38, an amino acid sequence comprising SEQ ID NO:40, and an amino acid sequence comprising SEQ ID NO:42. In some embodiments, the nucleic acid preparation comprises nucleic acid molecules encoding polypeptides encoding an amino acid sequence comprising SEQ ID NO:34, an amino acid sequence comprising SEQ ID NO:40, and an amino acid sequence comprising SEQ ID NO:42. In some embodiments, the nucleic acid preparation comprises nucleic acid molecules encoding polypeptides encoding an amino acid sequence comprising an amino acid sequence comprising SEQ ID NO:38, an amino acid sequence comprising SEQ ID NO:40, and an amino acid sequence comprising SEQ ID NO:42. In some embodiments, the nucleic acid preparation comprises nucleic acid molecules encoding polypeptides encoding an amino acid sequence comprising an amino acid sequence comprising SEQ ID NO:36, an amino acid sequence comprising SEQ ID NO:40, and an amino acid sequence comprising SEQ ID NO:42. In some embodiments, the nucleic acid preparation comprises nucleic acid molecules encoding polypeptides encoding an amino acid sequence comprising SEQ ID NO:34, an amino acid sequence comprising SEQ ID NO:38, and an amino acid sequence comprising SEQ ID NO:40. In some embodiments, the nucleic acid preparation comprises nucleic acid molecules encoding polypeptides encoding an amino acid sequence comprising an amino acid sequence comprising SEQ ID NO:34, an amino acid sequence comprising SEQ ID NO:36, and an amino acid sequence comprising SEQ ID NO:40. In some embodiments, the nucleic acid preparation comprises nucleic acid molecules encoding polypeptides encoding an amino acid sequence comprising an amino acid sequence comprising SEQ ID NO:36, an amino acid sequence comprising SEQ ID NO:38, and an amino acid sequence comprising SEQ ID NO:40.

In some embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a protein preparation comprising one or more reprogramming factors, culturing the non-hepatocyte cell under conditions suitable for reprogramming a non-hepatocyte cell to an induced hepatocyte, thereby producing an induced hepatocyte from the non-hepatocyte cell. In some embodiments, the protein preparation is a mixture of one or more reprograming factor proteins or polypeptides. In some embodiments, the protein preparation for use in producing an induced hepatocyte from a non-hepatocyte cell (e.g., for use in reprogramming a non-hepatocyte cell to an induced hepatocyte) comprises FOXA1, FOXA2, FOXA3, HNF1A, HNF4A, and GATA4. In some embodiments, the protein preparation for use in producing an induced hepatocyte from a non-hepatocyte cell (e.g., for use in reprogramming a non-hepatocyte cell to an induced hepatocyte) comprises CEBPA, GATA6, HHEX, HNF1B, HNF6A, FOXA1, FOXA2, FOXA3, GATA4, HNF1A, and HNF4A. In other embodiments, the protein preparation comprises FOXA1, FOXA2, FOXA3, HNF1A, and HNF4A. In other embodiments, the protein preparation comprises FOXA1, FOXA3, HNF1A, and HNF4A. In other embodiments, the protein preparation comprises FOXA1, FOXA2, HNF1A, and HNF4A. In other embodiments, the protein preparation comprises FOXA2, FOXA3, HNF1A, and HNF4A. In other embodiments, the protein preparation comprises FOXA1, HNF1A, and HNF4A. In other embodiments, the protein preparation comprises FOXA3, HNF1A, and HNF4A. In other embodiments, the protein preparation comprises FOXA2, HNF1A, and HNF4A. In other embodiments, the protein preparation comprises FOXA1, FOXA3, and HNF1A. In other embodiments, the protein preparation comprises FOXA1, FOXA2, and HNF1A. In other embodiments, the protein preparation comprises FOXA2, FOXA3, and HNF1A.

The present invention also provides a population of induced hepatocytes, wherein the induced hepatocytes are obtained using any of the methods and compositions described herein. In some embodiments, the population of induced hepatocytes provided by the present invention is a homogeneous population of induced hepatocytes. In some aspects, a homogeneous population of induced hepatocytes provided by the present invention is a population of cells wherein a significant portion of the cell population comprises induced hepatocytes, such as, for example, a population of cells in which more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99% of the cells in the population are induced hepatocytes produced by the present methods and compositions.

In other aspects, the present invention also provides nucleic acid compositions useful for reprogramming a non-hepatocyte cell to an induced hepatocyte. In some embodiments, the present invention provides nucleic acid compositions useful for reprogramming a human non-hepatocyte cell to a human induced hepatocyte. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA1, FOXA2, FOXA3, HNF1A, HNF4A, and GATA4. In some embodiments, the present invention provides a composition comprising a nucleic acid preparation useful for reprogramming a non-hepatocyte cell to an induced hepatocyte, wherein the nucleic acid preparation comprises nucleic acid molecules encoding CEBPA, GATA6, HHEX, HNF1B, HNF6A, FOXA1, FOXA2, FOXA3, GATA4, HNF1A, and HNF4A. In some embodiments, the composition comprising a nucleic acid preparation comprises nucleic acid molecules encoding FOXA1, FOXA2, FOXA3, HNF1A, and HNF4A. In some embodiments, the composition comprising a nucleic acid preparation comprises nucleic acid molecules encoding FOXA1, FOXA3, HNF1A, and HNF4A. In some embodiments, the composition comprising a nucleic acid preparation comprises nucleic acid molecules encoding FOXA1, FOXA2, HNF1A, and HNF4A. In some embodiments, the composition comprising a nucleic acid preparation comprises nucleic acid molecules encoding FOXA2, FOXA3, HNF1A, and HNF4A. In some embodiments, the composition comprising a nucleic acid preparation comprises nucleic acid molecules encoding FOXA1, HNF1A, and HNF4A. In some embodiments, the composition comprising a nucleic acid preparation comprises nucleic acid molecules encoding FOXA3, HNF1A, and HNF4A. In some embodiments, the composition comprising a nucleic acid preparation comprises nucleic acid molecules encoding FOXA2, HNF1A, and HNF4A. In some embodiments, the composition comprising a nucleic acid preparation comprises nucleic acid molecules encoding FOXA1, FOXA3, and HNF1A. In some embodiments, the composition comprising a nucleic acid preparation comprises nucleic acid molecules encoding FOXA1, FOXA2, and HNF1A. In some embodiments, the composition comprising a nucleic acid preparation comprises nucleic acid molecules encoding FOXA2, FOXA3, and HNF1A. In certain embodiments, the nucleic acid molecules encoding the reprogramming factors are RNA molecules or mRNA molecules.

In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, a nucleic acid sequence of SEQ ID NO:41, and a nucleic acid sequence comprising SEQ ID NO:43. In some embodiments, the composition comprising a nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41. In some embodiments, the composition comprising a nucleic acid preparation comprises nucleic acid molecules comprising a nucleic acid sequence comprising SEQ ID NO:33, a nucleic acid sequence comprising SEQ ID NO:37, a nucleic acid sequence comprising SEQ ID NO:39, and a nucleic acid sequence comprising SEQ ID NO:41. In some embodiments, the composition comprising a nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:39, and a nucleic acid sequence comprising SEQ ID NO:41. In some embodiments, the composition comprising a nucleic acid preparation comprises nucleic acid molecules comprising a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41. In some embodiments, the composition comprising a nucleic acid preparation comprises nucleic acid molecules comprising a nucleic acid sequence comprising SEQ ID NO:33, a nucleic acid sequence comprising SEQ ID NO:39, and a nucleic acid sequence comprising SEQ ID NO:41. In some embodiments, the composition comprising a nucleic acid preparation comprises nucleic acid molecules comprising a nucleic acid sequence comprising SEQ ID NO:37, a nucleic acid sequence comprising SEQ ID NO:39, and a nucleic acid sequence comprising SEQ ID NO:41. In some embodiments, the composition comprising a nucleic acid preparation comprises nucleic acid molecules comprising a nucleic acid sequence comprising SEQ ID NO:35, a nucleic acid sequence comprising SEQ ID NO:39, and a nucleic acid sequence comprising SEQ ID NO:41. In some embodiments, composition comprising a nucleic acid preparation comprises nucleic acid molecules comprising a nucleic acid sequence comprising SEQ ID NO:33, a nucleic acid sequence comprising SEQ ID NO:37, and a nucleic acid sequence comprising SEQ ID NO:39. In some embodiments, the composition comprising a nucleic acid preparation comprises nucleic acid molecules comprising a nucleic acid sequence comprising SEQ ID NO:33, a nucleic acid sequence comprising SEQ ID NO:35, and a nucleic acid sequence comprising SEQ ID NO:39. In some embodiments, the composition comprising a nucleic acid preparation comprises nucleic acid molecules comprising a nucleic acid sequence comprising SEQ ID NO:35, a nucleic acid sequence comprising SEQ ID NO:37, and a nucleic acid sequence comprising SEQ ID NO:39.

In various aspects of the methods provided herein, the non-hepatocyte cell is a human non-hepatocyte cell, and the resulting induced hepatocyte is a human induced hepatocyte. In some aspects of the methods provided herein, the non-hepatocyte cell is not a stem cell. In other aspects of the methods provided herein, the non-hepatocyte cell is not a pluripotent cell. In still other aspects of the methods provided herein, the non-hepatocyte cell is a somatic cell, including a human somatic cell.

In various aspects, the induced hepatocytes obtained by the methods of the present invention express hepatocyte genes, including albumin, α-fetoprotein, α1-anti-trypsin, cytokeratin 18, and delta-like 1.

The present invention also provides cell compositions comprising induced hepatocytes produced by the methods of the present invention. In some embodiments, various compositions of induced hepatocyte, such as cell cultures or cell populations which are substantially free of cells other than hepatocytes are provided by the present invention. In addition, compositions, such as cell cultures or cell populations that are enriched, isolated, or purified for hepatocytes are provided.

Compositions of induced hepatocytes provided by the present invention may comprise a homogeneous population of induced hepatocytes. In some aspects, the present invention provides a composition comprising induced hepatocytes, wherein the composition comprises a homogeneous population of induced hepatocytes. In some embodiments, the present invention provides a composition of induced hepatocytes, wherein the percentage of cells within the composition comprises about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% induced hepatocytes. In some embodiments, the percentage of cells in a composition comprising induced hepatocytes is 100%.

The present invention provides induced hepatocytes or populations of induced hepatocytes useful in various research and therapeutic applications. In some embodiments, the induced hepatocytes of the present invention are used as a model of various human diseases and disorders. In other embodiments, the induced hepatocytes of the present invention are used as models for hepatitis and other liver disease studies. The present invention provides induced hepatocytes useful for treating various degenerative liver diseases or inherited or acquired deficiencies of liver function. For example, the present invention provides methods for providing a cell-based therapy to a patient in need thereof, by administering to the patient a population of induced hepatocytes obtained by the methods disclosed herein. In certain embodiments, the patient in need has liver disease or a liver disorder such as, for example, liver fibrosis, cirrhosis, liver failure, hepatitis, liver cancer, etc.

The present invention also provides methods for screening a drug candidate for toxicity (e.g., hepatotoxicity) using the induced hepatocytes obtained by the methods described herein. In some embodiments, the present invention provides methods of screening a drug candidate for toxicity or hepatotoxicity by contacting a population of induced hepatocytes with the drug candidate and monitoring the induced hepatocytes for toxicity or hepatotoxicity, thereby identifying whether the drug candidate is toxic.

The present invention further provides a reprogramming cell culture media, wherein the reprogramming cell culture media comprises DMEM/F12+Glutamax media, 10% fetal bovine serum (FBS), 1% Insulin-Transferrin-Selenium, 1% MEM Non-Essential Amino Acids, 5 mM HEPES buffer, 20 ng/ml human hepatocyte growth factor (HGF), 20 ng/ml epidermal growth factor (EGF), 20 ng/ml fibroblast growth factor 2 (FGF2), 200 ng/mL B18R, and 0.1 μM dexamethasone. In various aspects of the present methods, the non-hepatocyte cells are cultured under conditions suitable for reprogramming a non-hepatocyte cell to an induced hepatocyte, wherein the cell culture media is the reprogramming media described above.

In other aspects, the present invention provides methods for identifying a factor that promotes reprogramming of non-hepatocyte cells into induced hepatocytes.

### BRIEF DESCRIPTION OF THE DRAWINGS

FIG. 1 sets forth data showing expression and cellular localization of green fluorescent protein (GFP) and nuclear GFP (NLS GFP) in human neonatal foreskin fibroblasts transfected with various amounts of mRNA encoding GFP or nuclear GFP.

FIG. 2 sets forth data showing reprogramming factor expression is maintained in human neonatal foreskin fibroblasts transfected daily for 9 days with a mixture of mRNAs encoding FOXA1, FOXA2, FOXA3, HNF4A, HNF1A, and GATA4.

FIG. 3 sets forth data showing protein expression and nuclear localization of FOXA1, FOXA2, FOXA3, HNF4A, HNF1A, and GATA4 in human neonatal foreskin fibroblasts transfected with a mixture of mRNAs encoding FOXA1, FOXA2, FOXA3, HNF4A, HNF1A, and GATA4.

FIG. 4 sets forth data showing the induction of albumin and α-fetoprotein (AFP) gene expression in human neonatal foreskin fibroblasts transfected with a mixture of mRNAs encoding FOXA1, FOXA2, FOXA3, HNF4A, HNF1A, and GATA4.

FIG. 5 sets forth data showing FOXA2 protein expression and nuclear localization correlated with α-fetoprotein (AFP) expression and cytoplasmic localization in human neonatal foreskin fibroblasts transfected with a mixture of mRNAs encoding FOXA1, FOXA2, FOXA3, HNF4A, HNF1A, and GATA4.

FIG. 6 sets forth data showing albumin protein expression and proper cytoplasmic localization in human neonatal foreskin fibroblasts transfected with a mixture of mRNAs encoding FOXA1, FOXA2, FOXA3, HNF1A, HNF4A, and GATA4.

FIG. 7 sets forth data showing the induction of alpha1-anti-trypsin (A1AT), cytokeratin 18 (CK18), and delta-like 1 protein (DLK1) gene expression in human neonatal foreskin fibroblasts transfected with a mixture of mRNAs encoding FOXA1, FOXA2, FOXA3, HNF1A, HNF4A, and GATA4.

FIG. 8 sets forth data showing bi-nucleation in human neonatal foreskin fibroblasts transfected with a mixture of mRNAs encoding FOXA1, FOXA2, FOXA3, HNF1A, HNF4A, and GATA4.

FIG. 9 sets forth data showing lipid droplets in human neonatal foreskin fibroblasts transfected with a mixture of mRNAs encoding FOXA1, FOXA2, FOXA3, HNF1A, HNF4A, and GATA4.

FIG. 10 sets forth data showing the induction of albumin and α-fetoprotein (AFP) gene expression in human fetal lung fibroblasts transfected with a mixture of mRNAs encoding FOXA1, FOXA2, FOXA3, HNF4A, HNF1A, and GATA4.

FIGS. 11A and 11B set forth data showing induction of albumin and α-fetoprotein (AFP) gene expression in human neonatal foreskin fibroblasts transfected with various mixtures of mRNAs encoding FOXA1, FOXA2, FOXA3, HNF1A, HNF4A, and GATA4.

FIGS. 12A and 12B set forth data showing induction of albumin and α-fetoprotein (AFP) gene expression in human neonatal foreskin fibroblasts transfected with various mixtures of mRNAs encoding FOXA1, FOXA2, FOXA3, HNF1A, and HNF4A.

FIGS. 13A and 13B set forth data showing induction of albumin and α-fetoprotein (AFP) gene expression in human neonatal foreskin fibroblasts transfected with various mixtures of mRNAs encoding FOXA1, FOXA3, HNF1A, and HNF4A.

FIGS. 14A and 14B set forth data showing induction of albumin and α-fetoprotein (AFP) gene expression in human neonatal foreskin fibroblasts transfected with mRNA encoding HNF1A and with mRNA encoding FOXA1, FOXA2, FOXA3, HNF4A, or GATA4.

FIG. 15 set forth the nucleic acid sequence of human FOXA1 (SEQ ID NO:33).

FIG. 16 sets forth the amino acid sequence of human FOXA1 (SEQ ID NO:34).

FIG. 17 sets forth the nucleic acid sequence of human FOXA2 (SEQ ID NO:35).

FIG. 18 sets forth the amino acid sequence of human FOXA2 (SEQ ID NO:36).

FIG. 19 sets forth the nucleic acid sequence of human FOXA3 (SEQ ID NO:37).

FIG. 20 sets forth the amino acid sequence of human FOXA3 (SEQ ID NO:38).

FIG. 21 sets forth the nucleic acid sequence of human HNF1A (SEQ ID NO:39).

FIG. 22 sets forth the amino acid sequence of human HNF1A (SEQ ID NO:40).

FIG. 23 sets forth the nucleic acid sequence of human HNF4A (SEQ ID NO:41).

FIG. 24 sets forth the amino acid sequence of human HNF4A (SEQ ID NO:42).

FIG. 25 sets forth the nucleic acid sequence of human GATA4 (SEQ ID NO:43).

FIG. 26 sets forth the amino acid sequence of human GATA4 (SEQ ID NO:44).

FIG. 27 sets forth data showing the induction of albumin and α-fetoprotein (AFP) gene expression in human fetal lung fibroblasts transfected with a mixture of mRNAs encoding C/EBPα, FOXA1, FOXA2, FOXA3, GATA4, GATA6, HHEX, HNF1A, HNF1B, HNF4A, and HNF6A.

FIG. 28 sets forth data showing the induction of albumin and α-fetoprotein (AFP) gene expression in human fetal lung fibroblasts transfected with a mixture of mRNAs encoding C/EBPα, FOXA1, FOXA2, FOXA3, GATA4, GATA6, HHEX, HNF1A, HNF1B, HNF4A, and HNF6A (11TF) or with a mixture of mRNAs encoding FOXA1, FOXA2, FOXA3, HNF1A, HNF4A, and GATA4 (6TF).

FIGS. 29A and 29B set forth data showing expression levels of various hepatocyte genes in human neonatal foreskin fibroblasts (FIG. 29A) and human fetal lung fibroblasts (FIG. 29B) transfected with a mixture of mRNAs encoding FOXA1, FOXA2, FOXA3, HNF1A, HNF4A, and GATA4 compared to that observed in primary hepatocytes.

FIG. 30 sets forth data showing expression levels of various cell surface markers in human fibroblasts transfected with a mixture of mRNAs encoding FOXA1, FOXA2, FOXA3, HNF1A, HNF4A, and GATA4.

FIG. 31 sets forth data showing the effect of various concentrations of dexamethasone on albumin gene expression in human fibroblasts transfected with a mixture of mRNAs encoding C/EBPα, FOXA1, FOXA2, FOXA3, GATA4, GATA6, HHEX, HNF1A, HNF1B, HNF4A, and HNF6A (11TF) or with a mixture of mRNAs encoding FOXA1, FOXA2, FOXA3, HNF1A, HNF4A, and GATA4 (6TF).

FIG. 32 sets forth data showing the ability of various transcription factors encoding FOXA1, FOXA2, FOXA3, HNF1A, HNF4A, and GATA4 to induce expression of transcription factors encoding FOXA1, FOXA2, FOXA3, HNF1A, HNF4A, and GATA4 in human fibroblasts.

FIG. 33 sets forth data showing gene cluster analysis of human fibroblasts transfected with various combinations of transcription factors encoding FOXA1, FOXA2, FOXA3, HNF1A, HNF4A, and GATA4 compared to that observed in human hepatocytes.

FIGS. 34A and 34B set forth data showing gene expression levels of 33 hepatocyte genes in human fetal fibroblasts and in human embryonic stem cells, respectively, transfected with 6TF mRNA mixture compared to that observed in primary hepatocytes.

FIGS. 35A and 35B set forth data showing comparison of global gene transcriptome expression similarity and global small RNA expression similarity, respectively, plotted as log 2 ratios human fetal fibroblasts transfected with 6TF mRNA mixture, 11TF mRNA mixture, and vehicle control by RNA sequencing.

FIG. 36 sets forth data showing log 2 ratios of the top 25 up-regulated genes in human fetal hepatocytes transfected with 6TF mRNA mixture above vehicle-treated control cells (red, liver associated genes; blue, other endodermal genes; green, histone genes).

FIG. 37 sets forth data showing up-regulation of histones in human fetal fibroblasts transfected with 6TF mRNA mixture compared to that observed in vehicle-treated control cells, plotted as RPKM log 2.

FIG. 38 sets forth data showing log 2 ratios of tissue-specific genes which are up-regulated or down-regulated more than 2-fold above control of human fetal fibroblasts transfected with 6TF mRNA mixture.

### DETAILED DESCRIPTION OF THE INVENTION

The present invention provides, inter alia, methods and compositions useful for efficient reprogramming of non-hepatocyte cells to induced hepatocytes, populations of induced hepatocytes, compositions comprising induced hepatocytes, and uses thereof. The methods and compositions described herein are useful for reprogramming a starting cell population (e.g., a non-hepatocyte cell population) to a population of induced hepatocytes with high efficiency. The present invention also comprises compositions comprising nucleic acids encoding reprogramming factors useful for reprogramming non-hepatocyte cells to induced hepatocytes. The present invention further comprises compositions comprising reprogramming factor polypeptides useful for reprogramming non-hepatocyte cells to induced hepatocytes. In various aspects, the non-hepatocyte cells are of human origin, and the induced hepatocytes are human induced hepatocytes.

It is understood that reference to a population of induced hepatocytes described herein contemplates and includes an isolated population of induced hepatocytes.

### General Methods

The practice of the present invention will employ, unless otherwise indicated, conventional techniques of cell biology, cell culture, molecular biology (including recombinant techniques), microbiology, biochemistry, and immunology, which are known and available to one of skill in the art. Such techniques are described in the literature, such as, Molecular Cloning: A laboratory Manual, third edition (Sambrook et al., 2001) Cold Spring Harbor Press; Oligonucleotide Synthesis (P. Herdewijn, ed., 2004); Animal Cell Culture (R. I. Freshney, ed., 1987); Methods in Enzymology (Academic Press, Inc.); Current Protocols in Molecular Biology (F. M. Ausubel et al., eds., 1987); PCR: The Polymerase Chain Reaction (Mullis et al., eds., 1994); Current Protocols in Immunology (J. E. Coligan et al., eds., 1991); and Short Protocols in Molecular Biology (Wiley and Sons, 1999).

Unless defined otherwise, all technical and scientific terms used herein have the same meaning as is commonly understood by one of ordinary skill in the art to which the invention belongs.

### DEFINITIONS

The term “non-hepatocyte cell” encompasses a cell of non-hepatocyte origin or of a non-hepatocyte differentiation state or lineage. Non-hepatocyte cells may include somatic cells. Non-limiting examples of non-hepatocyte cells include fibroblasts, bone marrow cells, cord blood cells, endothelial cells (e.g., human umbilical vein endothelial cells), keratinocytes, mesangial cells, embryonic stem cells, etc.

The term “induced hepatocyte” encompasses a hepatocyte (e.g., a hepatocyte cell) that arises or is obtained from a non-hepatocyte cell by experimental manipulation. Induced hepatocytes express markers indicative of cells of hepatocyte lineage, such as, for example, albumin, α-fetoprotein, α1-anti-trypsin, etc. Induced hepatocytes may have characteristics of functional hepatocytes, including functional immature hepatocytes, hepatocyte precursors, or mature hepatocytes, such as, for example, bi-nucleation, neutral lipid droplets, glycogen storage, and expression of, for example, albumin, α-fetoprotein, alpha-1-antitrypsin, cytokeratin 18, Delta-like 1 (DLK1), CD133, N-cadherin (NCAD), etc.

The term “reprogramming” refers to the process of altering the differentiation state of a cell to a different differentiation state. Reprogramming also refers to the process of altering the differentiation state of terminally-differentiated cell (e.g., a terminally-differentiated somatic cell) to a different differentiation state.

The term “somatic cell” encompasses any cell in an organism that cannot give rise to all types of cells in an organism, i.e., a cell that is not pluripotent. In other words, somatic cells are cells that have differentiated sufficiently that they will not naturally generate cells of all three germ layers of the body, i.e., ectoderm, mesoderm, and endoderm.

The term “introducing” as used herein refers to the process of bringing an agent, a protein, a polypeptide, or a nucleic acid into a living cell, including, but not limited to, by an introducing means as described herein.

The term “contacting” as used herein refers to the process of bringing an agent, a protein, a polypeptide, or a nucleic acid in contact with living cell, including, but not limited to, by a contacting means as described herein.

The term “reprogramming factor” as used herein refers to a protein, polypeptide, polynucleotide, nucleic acid, or other biomolecule that, when used alone or in combination with other factors or conditions, causes a change in the state of differentiation of a cell in which the reprogramming factor is introduced or expressed. In some embodiments of the present invention, a reprogramming factor is a protein or polypeptide that is encoded by a nucleic acid (e.g., mRNA), for which the nucleic acid encoding the reprogramming factor is introduced into a cell, thereby generating a cell that exhibits a changed state of differentiation compared to the cell in which the nucleic acid encoding the reprogramming factor was not introduced. In certain instances, a reprogramming factor is a transcription factor.

Reference to culturing a non-hepatocyte cell “under conditions suitable for reprogramming a non-hepatocyte cell to an induced hepatocyte” refers to growth conditions that will support the growth and health of the target cell (i.e., induced hepatocyte) and the starting cell type (e.g., neonatal fibroblast, fetal fibroblasts, keratinocyte, mesenchymal stem cell, hematopoietic stem cell, etc.).

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, “an effective amount” refers to an amount effective to achieve a goal (e.g., the desired goal) of any of the methods described herein.

As used herein, the singular form of “a”, “an”, and “the” includes the plural references unless indicated otherwise.

Reference to “about” a value or parameter herein refers to the usual error range for the respective value readily known to the skilled person in this technical field. Reference to “about” a value or parameter herein includes (and describes) aspects that are directed to that value or parameter, per se. For example, description referring to “about X” includes description of “X.”

### Methods of Producing Induced Hepatocytes

The present invention provides methods for efficient reprogramming of non-hepatocyte cells to induced hepatocytes, populations of induced hepatocytes produced by the present methods, compositions comprising induced hepatocytes produced by the present methods, and uses thereof. The methods and compositions described herein are useful for reprogramming a starting cell or starting cell population (e.g., a non-hepatocyte cell or non-hepatocyte cell population) to an induced hepatocyte or a population of induced hepatocytes with high efficiency. In some embodiments, the starting cell or starting cell population is a human non-hepatocyte cell or a human non-hepatocyte cell population (e.g, a non-hepatocyte cell of human origin), and the resulting induced hepatocytes are human induced hepatocytes or a population of human induced hepatocytes.

The methods of the present invention can be practiced using a starting cell population of non-hepatocyte cells of various types, including, for example, fibroblasts, mesenchymal cells, keratinocytes, hematopoietic cells, etc. The non-hepatocyte cells can be of adult or non-adult origin, including neonatal, fetal, and embryonic non-hepatocyte cells. Starting cells useful for the present methods also include primary embryonic cells, fetal cells, neonatal cells, somatic cells, blood cells (e.g., hematopoietic cells), non-adult cells, as well as cells derived from adult tissue, umbilical cord tissue, placental tissue, bone marrow, and other cell sources. In certain embodiments, the starting cell or starting cell population of non-hepatocyte cells are of human origin (i.e., human non-hepatocyte cells), and the induced hepatocytes produced therefrom according to the present methods are human induced hepatocytes.

The present inventors have identified key reprogramming factors that are sufficient to reprogram human non-hepatocyte cells to human induced hepatocytes. The identified reprogramming factors useful for reprogramming human non-hepatocyte cells to human induced hepatocytes include forkhead box protein A1 (FOXA1), also known as hepatocyte nuclear factor 3-alpha (HNF3A); forkhead box protein A2 (FOXA2), also known as hepatocyte nuclear factor 3-beta (HNF3B) or transcription factor 3B (TCF3B); forkhead box protein A3 (FOXA3), also known as hepatocyte nuclear factor 3-gamma (HNF3G) or transcription factor 3G (TCF3G); hepatocyte nuclear factor 1 homeobox A (HNF1A); hepatocyte nuclear factor 4 alpha (HNF4A), also known as nuclear receptor subfamily 2, group A, member 1 (NR2A1); and transcription factor GATA binding protein 4 (GATA4). Other identified reprogramming factors useful for reprogramming human non-hepatocyte cells to human induced hepatocytes include CEBPA, GATA6, HHEX, HNF1B, and HNF6A. The present invention discloses that various combinations of the reprogramming factors are important for reprogramming human non-hepatocyte cells to human induced hepatocytes.

In some embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding one or more reprogramming factors, and culturing the non-hepatocyte cell under conditions suitable for reprogramming a non-hepatocyte cell to an induced hepatocyte, thereby producing an induced hepatocyte from the non-hepatocyte cell. In some embodiments, the nucleic acid preparation is a mixture of nucleic acids (e.g., a nucleic acid preparation comprising a mixture of nucleic acid molecules) encoding one or more reprograming factors. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA1, FOXA2, FOXA3, HNF1A, HNF4A, and GATA, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, a nucleic acid sequence comprising SEQ ID NO:41, and a nucleic acid sequence comprising SEQ ID NO:43, and culturing the non-hepatocyte cell under conditions suitable for reprogramming, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:36, an amino acid sequence comprising SEQ ID NO:38, an amino acid sequence comprising SEQ ID NO:40, an amino acid sequence comprising SEQ ID NO:42, and an amino acid sequence comprising SEQ ID NO:44, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA1, FOXA2, FOXA3, HNF1A, and HNF4A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:36, an amino acid sequence comprising SEQ ID NO:38, an amino acid sequence comprising SEQ ID NO:40, and an amino acid sequence comprising SEQ ID NO:42, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA1, FOXA3, HNF1A, and HNF4A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:38, an amino acid sequence comprising SEQ ID NO:40, and an amino acid sequence comprising SEQ ID NO:42, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:37, a nucleic acid sequence comprising SEQ ID NO:39, and a nucleic acid sequence comprising SEQ ID NO:41, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA1, FOXA2, HNF1A, and HNF4A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:36, an amino acid sequence comprising SEQ ID NO:40, and an amino acid sequence comprising SEQ ID NO:42, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:39, and a nucleic acid sequence comprising SEQ ID NO:41, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA2, FOXA3, HNF1A, and HNF4A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:36, an amino acid sequence comprising SEQ ID NO:38, an amino acid sequence comprising SEQ ID NO:40, and an amino acid sequence comprising SEQ ID NO:42, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA1, HNF1A, and HNF4A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:40, and an amino acid sequence comprising SEQ ID NO:42, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:39, and a nucleic acid sequence comprising SEQ ID NO:41, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA3, HNF1A, and HNF4A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising an amino acid sequence comprising SEQ ID NO:38, an amino acid sequence comprising SEQ ID NO:40, and an amino acid sequence comprising SEQ ID NO:42, culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:37, a nucleic acid sequence comprising SEQ ID NO:39, and a nucleic acid sequence comprising SEQ ID NO:41, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA2, HNF1A, and HNF4A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:36, an amino acid sequence comprising SEQ ID NO:40, and an amino acid sequence comprising SEQ ID NO:42, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:35, a nucleic acid sequence comprising SEQ ID NO:39, and a nucleic acid sequence comprising SEQ ID NO:41, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA1, FOXA3, and HNF1A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:38, and an amino acid sequence comprising SEQ ID NO:40, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:37, and a nucleic acid sequence comprising SEQ ID NO:39, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA1, FOXA2, and HNF1A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:34, an amino acid sequence comprising SEQ ID NO:36, and an amino acid sequence comprising SEQ ID NO:40, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:33, a nucleic acid sequence comprising SEQ ID NO:35, and a nucleic acid sequence comprising SEQ ID NO:39, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding FOXA2, FOXA3, and HNF1A, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding polypeptides encoding an amino acid sequence comprising SEQ ID NO:36, an amino acid sequence comprising SEQ ID NO:38, and an amino acid sequence comprising SEQ ID NO:40, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules comprising a nucleic acid sequence comprising SEQ ID NO:35, a nucleic acid sequence comprising SEQ ID NO:37, and a nucleic acid sequence comprising SEQ ID NO:39, and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a nucleic acid preparation, wherein the nucleic acid preparation comprises nucleic acid molecules encoding, CEBPA, GATA6, HHEX, HNF1B, HNF6A, FOXA1, FOXA2, FOXA3, GATA4, HNF1A, and HNF4A and culturing the non-hepatocyte cell under conditions suitable for reprogramming a non-hepatocyte cell to an induced hepatocyte, thereby producing an induced hepatocyte from the non-hepatocyte cell. In particular embodiments, the non-hepatocyte cell is a human non-hepatocyte cell, and the resulting induced hepatocyte is a human induced hepatocyte.

In other embodiments, the present invention provides a method for producing an induced hepatocyte from a non-hepatocyte cell (e.g., a method for reprogramming a non-hepatocyte cell to an induced hepatocyte), the method comprising providing a non-hepatocyte cell, introducing into the non-hepatocyte cell a protein preparation comprising one or more reprogramming factors, culturing the non-hepatocyte cell under conditions suitable for reprogramming a non-hepatocyte cell to an induced hepatocyte, thereby producing an induced hepatocyte from the non-hepatocyte cell. In some embodiments, the protein preparation is a mixture of one or more reprograming factor proteins or polypeptides.

In some embodiments, the protein preparation for use in producing an induced hepatocyte from a non-hepatocyte cell (e.g., for use in reprogramming a non-hepatocyte cell to an induced hepatocyte) comprises FOXA1, FOXA2, FOXA3, HNF1A, HNF4A, and GATA4. In other embodiments, the protein preparation comprises CEBPA, GATA6, HHEX, HNF1B, HNF6A, FOXA1, FOXA2, FOXA3, GATA4, HNF1A, and HNF4A. In other embodiments, the protein preparation comprises FOXA1, FOXA2, FOXA3, HNF1A, and HNF4. In other embodiments, the protein preparation comprises FOXA1, FOXA3, HNF1A, and HNF4A. In other embodiments, the protein preparation comprises FOXA1, FOXA2, HNF1A, and HNF4A. In other embodiments, the protein preparation comprises FOXA2, FOXA3, HNF1A, and HNF4A. In other embodiments, the protein preparation comprises FOXA1, HNF1A, and HNF4A. In other embodiments, the protein preparation comprises FOXA3, HNF1A, and HNF4A. In other embodiments, the protein preparation comprises FOXA2, HNF1A, and HNF4A. In other embodiments, the protein preparation comprises FOXA1, FOXA3, and HNF1A. In other embodiments, the protein preparation comprises FOXA1, FOXA2, and HNF1A. In other embodiments, the protein preparation comprises FOXA2, FOXA3, and HNF1A. In particular embodiments, the protein preparation is useful for reprogramming a non-hepatocyte cell to an induced hepatocyte, wherein the non-hepatocyte cell is a human non-hepatocyte cell, and the resulting induced hepatocyte is a human induced hepatocyte.

In some aspects, the reprogramming factors useful in the methods described herein may comprise one, two, three, four, five, or six reprogramming factors, wherein the reprogramming factors are selected from the group consisting of FOXA1, FOXA2, FOXA3, HNF1A, HNF4A, and GATA4. In other aspects, the reprogramming factors are selected from the group consisting of CEBPA, GATA6, HHEX, HNF1B, HNF6A, FOXA1, FOXA2, FOXA3, GATA4, HNF1A, and HNF4A.

In some embodiments, the reprogramming factors useful for producing an induced hepatocyte from a non-hepatocyte cell comprise FOXA1, FOXA2, FOXA3, HNF1A, HNF4A, and GATA4. In some embodiments, the reprogramming factors useful for producing an induced hepatocyte from a non-hepatocyte cell comprise CEBPA, GATA6, HHEX, HNF1B, HNF6A, FOXA1, FOXA2, FOXA3, GATA4, HNF1A, and HNF4A. In some embodiments, the reprogramming factors useful for producing an induced hepatocyte from a non-hepatocyte cell comprise FOXA1, FOXA2, FOXA3, HNF1A, and HNF4A. In some embodiments, the reprogramming factors useful for producing an induced hepatocyte from a non-hepatocyte cell comprise FOXA1, FOXA3, HNF1A, and HNF4A; FOXA1, FOXA2, HNF1A, and HNF4A; or FOXA2, FOXA3, HNF1A, and HNF4A. In some embodiments, the reprogramming factors useful for producing an induced hepatocyte from a non-hepatocyte cell comprise FOXA1, HNF1A, and HNF4A. In some embodiments, the reprogramming factors useful for producing an induced hepatocyte from a non-hepatocyte cell comprise FOXA3, HNF1A, and HNF4A. In some embodiments, the reprogramming factors useful for producing an induced hepatocyte from a non-hepatocyte cell comprise FOXA2, HNF1A, and HNF4A. In some embodiments, the reprogramming factors useful for producing an induced hepatocyte from a non-hepatocyte cell comprise FOXA1, FOXA3, and HNF1A. In some embodiments, the reprogramming factors useful for producing an induced hepatocyte from a non-hepatocyte cell comprise FOXA1, FOXA2, and HNF1A. In some embodiments, the reprogramming factors useful for producing an induced hepatocyte from a non-hepatocyte cell comprise FOXA2, FOXA3, and HNF1A. In various embodiments, the non-hepatocyte cell is a human non-hepatocyte cell, and the induced hepatocyte is a human induced hepatocyte.

In certain embodiments, a nucleic acid molecule encoding a reprograming factor for use in the present methods is an mRNA molecule. In some embodiments, the mRNA encoding a reprogramming factor is purified mRNA. In some embodiments, the mRNA is produced by in vitro transcription from template DNA encoding a reprogramming factor. In other embodiments, a nucleic acid molecule encoding a reprogramming factor for use in the present methods is a DNA molecule. In some embodiments, the DNA encoding a reprogramming factor is genomic DNA. In other embodiments, the DNA encoding a reprogramming factor is cDNA. In other embodiments, the nucleic acid encoding a reprogramming factor for use in the present methods is contained within a plasmid, a vector, a virus, etc.

The present methods for producing induced hepatocytes from non-hepatocyte cells are not limited to in vitro methodologies or procedures. The present invention also provides in vivo methods for producing induced hepatocytes from non-hepatocyte cells Such in vivo methods include (but are not limited to) the use of retrovirus-based means of introducing nucleic acids encoding one or more reprogramming factors for in vivo administration, and for in vivo delivery of mRNA molecules (e.g., in vitro transcribed mRNA molecules) encoding one or more reprogramming factors. (See, e.g., Qian et al., (2012) Nature 485:593-598 and Song et al., (2012) Nature 485:599-604.) Such methods for producing induced hepatocytes from non-hepatocyte cells in vivo may be applied to the treatment of various diseases and disorders, such as, for example, fibrotic or cirrhotic liver disease by reprogramming endogenous fibroblasts or stellate cells to induced hepatocytes.

In some embodiments the methods of the present invention, the step of introducing a nucleic acid or nucleic acid preparation into a non-hepatocyte cell comprises delivering the nucleic acid into the cell with a transfection agent (e.g., TRANSIT mRNA transfection reagent). However, the invention is not limited by the nature of the means by which a nucleic acid or nucleic acid preparation is introduced into a non-hepatocyte cell, nor is the invention limited by the nature of the transfection method utilized. Any transfection process known, or identified in the future, that is able to deliver or introduce nucleic acid molecules into cells in vitro or in vivo is contemplated herein, including methods that deliver the nucleic acid into cells in culture or in a life-supporting medium, whether such cells comprise isolated cells or cells comprising eukaryotic tissue or organ, or methods that deliver nucleic acid in vivo into cells in an organism, such as a human. Useful transfection reagents include a lipid (e.g., liposomes, micelles, etc.), a nanoparticle or nanotube, a cationic compound (e.g., polyethylene imine or PEI), etc. Other transfection methods can be used, including the use of an electric current to deliver the nucleic acid into the cell (e.g., by electroporation) or the use of bolistics methods to deliver the nucleic acid into the cell (e.g., a “gene gun” or biolistic particle delivery system).

The present invention provides methods for reprogramming a non-hepatocyte cell to an induced hepatocyte by introducing into the non-hepatocyte cell a nucleic acid preparation, wherein the nucleic acid preparation comprises nucleic acid molecules encoding reprogramming factors. In certain aspects of the present methods, introducing nucleic acid molecules encoding a reprogramming factor to non-hepatocyte cells can be performed at various intervals, frequency, and periods of time. For example, nucleic acid molecules can be introduced into non-hepatocyte cells at a frequency of at least once daily, at least once every other day, at least once every third day, at least once every fourth day, at least once every fifth day, at least once every sixth day, at least once every seventh day, at least once every eight day, at least once every ninth day, etc. The introduction of nucleic acid molecules into non-hepatocyte cells can occur at any of these frequencies, and for duration sufficient enough to produce an induced hepatocyte, such as, for example, for 1 day, for 2 days, for 3 days, for 4 days, for 5 days, for 6 days, for 7 days, for 8 days, for 9 days, etc. The frequency and duration of introducing nucleic acid encoding a transcription factor preparation into a non-hepatocyte cell in order to effectively produce induced hepatocytes can be readily determined by one of ordinary skill in the art.

The present invention also shows expression of reprogramming factors in non-hepatocyte cells correlated with the amount of mRNA introduced (e.g., transfected) into the non-hepatocyte cells, indicating that the degree of expression of reprogramming factors encoded by individual mRNAs can be adjusted in a dose-dependent manner. The amount of nucleic acid (e.g., mRNA) encoding one or more transcription factors for use in reprogramming a non-hepatocyte cell in order to effectively produce induced hepatocytes can be readily determined and adjusted by one of ordinary skill in the art. The amount of nucleic acid needed to effectively reprogram a non-hepatocyte cell to an induced hepatocyte may vary based on cell type, cell number, cell culture conditions, etc. It is well within the ordinary skill of one in the art to experimentally determine the amount of nucleic acid (e.g., mRNA) to introduce into the non-hepatocyte cells to effectively produce an induced hepatocyte. The methods of the present invention are not limited to the use of any specific amount of nucleic acid to introduce into a non-hepatocyte cell in order to produce an induced hepatocyte.

The present methods provide various advantages for reprogramming cells over methodologies previously described. In certain aspects of the present invention, the introduction of mRNA into a non-hepatocyte in order to produce an induced hepatocyte provides various advantages over introducing, for example, DNA, plasmids, vectors, viruses, etc., into a non-hepatocyte cell. One advantage of the methods provided by the present invention is that the introduction of nucleic acid into a non-hepatocyte cell does not result in incorporation of the nucleic acid into the genome of the cell. Another advantage of the instant methods is that translation of the nucleic acid (e.g., mRNA) occurs soon after the nucleic acid is introduced into a cell; therefore the expression and appearance of the encoded product is rapid. Another advantage of the instant methods is that the amount of reprogramming factor protein expressed from the nucleic acid (e.g., mRNA) can be adjusted by delivering more or less nucleic acid to the cell. Yet another advantage of the instant methods is that repeated delivery of nucleic acid to a cell does not induce an immune response. Another advantage of the present methods is a lack of the requirement that the mRNA molecules introduced into the non-hepatocyte cells enter the nucleus of the cells, providing more rapid and efficient reprogramming.

According to one embodiment of the present invention, introduction of nucleic acid molecules into a population of non-hepatocyte cells occurs without genetic modification of the cells, such as, for example, without nucleic acid incorporation into the genome of the cells. Lack of or without genetic modification in this context will be understood to mean the absence of heterologous nucleic acid sequences (e.g., those nucleic acid sequences encoding transcription factors) stably introduced in the genome of the induced hepatocyte but not found in the starting non-hepatocyte cells. By heterologous is meant that the nucleic acid in question has been introduced into the cell or an ancestor thereof using genetic engineering or manipulation. A heterologous nucleic acid may normally be absent from the cell of that type or may be additional to an endogenous gene of the cell (e.g., an additional copy of a reprogramming factor, where the endogenous copy has been inactivated or silenced) but in each case the heterologous nucleic acid is introduced by human intervention.

The present invention also provides methods for producing an induced hepatocyte from a non-hepatocyte cell using methods and/or agents that modulate the expression or activity of one or more reprogramming factors identified herein. Modulation of the expression or activity of one or more reprogramming factors can be by direct activation (e.g., direct activation of the expression or activity one or more reprogramming factors identified herein) or by indirect activation (e.g., indirect activation of the expression or activity of one or more reprogramming factors identified herein), wherein the direct activation or the indirect activation produces an induced hepatocyte from a non-hepatocyte cell.

Direct or indirect activation as used herein can occur in which an agent or combination of agents induces, activates, or increases expression or activity of one or more of the reprogramming factors described herein (i.e., induces, activates, or increases the expression or activity of one or more of FOXA1, FOXA2, FOXA3, HNF1A, HNF4A, and GATA4). Such direct or indirect activation can occur by contacting a non-hepatocyte cell with one or more agents effective at inducing, activating, or increasing the expression or activity of one or more of FOXA1, FOXA2, FOXA3, HNF1A, HNF4A, and GATA4. Such agents can include any agent that modulates the expression or activity of a reprogramming factor, such as, for example, small molecules, growth factors, and other agents.

Recent reports have shown that the small molecule kenpaullone was effective at reprogramming murine fibroblasts to induced pluripotent cells, essentially replacing the reprogramming factor Klf4. (See Lyssiotis et al., (2009) PNAS 106:8912-8917.) This report indicated that non-transcription factor agents, which effectively reprogram cells of one differentiation state to another, could be identified. The present invention provides methods for identifying one or more agents capable of direct or indirect reprogramming of non-hepatocyte cells to induced hepatocytes. For each of these methods, the starting cell culture or cell population of non-hepatocyte cells is contacted with one or more candidate agents, and the cell or cell population is monitored for reprogramming to induced hepatocytes (e.g., by detecting, measuring, or quantitating gene or protein expression, activity, or phenotypic changes indicative of an induced hepatocyte). Other methods for determining whether one or more candidate agents can affect the direct or indirect reprogramming (or differentiation) of non-hepatocyte cells to induced hepatocytes have been described previously. (See, e.g., WO 2007/127454.)

### Population of Hepatocytes

The present invention also provides a population of induced hepatocytes, wherein the induced hepatocytes are obtained using any of the methods and compositions described herein. In certain embodiments, the population of induced hepatocytes provided by the present invention is a homogeneous population of induced hepatocytes. In some aspects, a homogeneous population of induced hepatocytes provided by the present invention is a population of cells wherein a significant portion of the cell population comprises induced hepatocytes. As used herein, a significant portion refers to a population of cells in which more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99% of the cells in the population are induced hepatocytes.

In some instances, it may be desirable to enrich for induced hepatocytes from a population of cells comprising both non-hepatocyte cells and induced hepatocytes. Induced hepatocytes of the present invention can be enriched from a mixture of both non-hepatocyte cells and induced hepatocytes by various techniques and methodologies for enriching cells well-known in the art. For example, cell sorting methodologies, such as fluorescence activated cell sorting (FACS), can be used to effectively enrich for induced hepatocytes.

The presence of any one or more of hepatocyte cell markers can be used to distinguish, identify, and enrich for an induced hepatocyte or a population of induced hepatocytes obtained by the methods of the present invention from a population of non-hepatocyte cells. Hepatocyte cell markers can be detected using standard methods known and available in the art, including, but not limited to, immunohistochemistry, flow cytometry, fluorescence imaging, PCR, Western blot, northern blot, etc. For example, various hepatocyte cell markers can be cell surface markers expressed on induced hepatocytes but not expressed on non-hepatocyte cells, or cell surface markers specific to cells of hepatocyte lineage. Cell surface hepatocyte markers include, for example, E-cadherin, DLK1, CD133, etc. Hepatocyte cell markers are well-known to one of skill in the art. (See, e.g., http://www.stembook.org/node/512.) Hepatocyte cell markers can be measured at different time points of culture of the cells or following the introduction of reprogramming factors (or nucleic acid encoding reprogramming factors) to the non-hepatocyte cells, such as, for example, at 1 day, 2 days, 3 days, 4 days, 5 days, 6 days, 7 days, 8 days, 9 days, 10 days, or more after one or more transfection rounds.

With respect to certain embodiments of the present invention, induced hepatocytes can be enriched, isolated, or purified for use in various research and therapeutic applications. For example, induced hepatocytes of the present inventions can be enriched, isolated, or purified by contacting a cell population that includes induced hepatocytes of the present invention with a reagent that binds to or otherwise interacts with a marker which is expressed in or on the induced hepatocyte but which is not substantially expressed in or on non-hepatocyte cells, and separating cells bound by the reagent from cells that are not bound by the reagent. In some embodiments for enrichment, isolation, or purification of induced hepatocytes, the marker can be any marker that is expressed in or on the induced hepatocytes but not substantially expressed in or on non-hepatocyte cells. Such markers include, but are not limited to, albumin, α-fetoprotein, alpha-1-antitrypsin, cytokeratin 18, DLK1, CD133, N-cadherin (NCAD), etc.

Any population of induced hepatocytes obtained using the methods described herein can be cryogenically preserved in the form of a cell bank of induced hepatocytes. Such cell banks can be thawed for subsequent therapeutic or experimental use. The cell banks of induced hepatocytes of the present invention can be prepared for cryogenic storage and cryogenically stored using methods known and available to one of skill in the art.

### Nucleic Acid Compositions

The present invention also provides nucleic acid compositions useful for reprogramming a non-hepatocyte cell to an induced hepatocyte. In some embodiments, the present invention provides nucleic acid compositions useful for reprogramming a human non-hepatocyte cell to a human induced hepatocyte. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA1, FOXA2, FOXA3, HNF1A, HNF4A, and GATA4. In some embodiments, the present invention provides a composition comprising a nucleic acid preparation useful for reprogramming a non-hepatocyte cell to an induced hepatocyte, wherein the nucleic acid preparation comprises nucleic acid molecules encoding CEBPA, GATA6, HHEX, HNF1B, HNF6A, FOXA1, FOXA2, FOXA3, GATA4, HNF1A, and HNF4A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA1, FOXA2, FOXA3, HNF1A, and HNF4A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA1, FOXA3, HNF1A, and HNF4A; or, wherein the nucleic acid preparation comprises nucleic acid molecules encoding FOXA1, FOXA2, HNF1A, and HNF4A; or wherein the nucleic acid preparation comprises nucleic acid molecules encoding FOXA2, FOXA3, HNF1A, and HNF4A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA1, HNF1A, and HNF4A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA3, HNF1A, and HNF4A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA2, HNF1A, and HNF4A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA1, FOXA3, and HNF1A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA1, FOXA2, and HNF1A. In some embodiments, the present invention provides a composition comprising a nucleic acid preparation useful for reprogramming a non-hepatocyte cell to an induced hepatocyte, wherein the nucleic acid preparation comprises nucleic acid molecules encoding FOXA2, FOXA3, and HNF1A. In various embodiments, the nucleic acid molecules useful for reprogramming a non-hepatocyte cell to an induced hepatocyte are RNA molecules, mRNA molecules, DNA molecules, cDNA molecules, etc. In particular embodiments, the nucleic acid molecules encoding the reprogramming factors identified above are purified or substantially purified nucleic acid molecules. In other particular embodiments, the nucleic acid molecules comprise modified nucleotides or modified nucleosides, such as, for example, mRNA molecules transcribed using modified nucleosides such as methyl-CTP and pseudo-UTP. Methods for preparing modified RNA molecules have been described previously. (See, e.g., International Application Publication Nos. WO 2011/071931 and WO 2007/024708, and U.S. Application Publication No. US 2009/0286852, the contents of which are incorporated by reference herein in their entirety.)

In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, a nucleic acid sequence of SEQ ID NO:41, and a nucleic acid sequence comprising SEQ ID NO:43. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:37, a nucleic acid sequence comprising SEQ ID NO:39, and a nucleic acid sequence comprising SEQ ID NO:41. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:39, and a nucleic acid sequence comprising SEQ ID NO:41. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:35, a nucleic acid sequence comprising SEQ ID NO:37, a nucleic acid sequence comprising SEQ ID NO:39, and a nucleic acid sequence comprising SEQ ID NO:41. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:39, and a nucleic acid sequence comprising SEQ ID NO:41. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:37, a nucleic acid sequence comprising SEQ ID NO:39, and a nucleic acid sequence comprising SEQ ID NO:41. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:35, a nucleic acid sequence comprising SEQ ID NO:39, and a nucleic acid sequence comprising SEQ ID NO:41. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:37, and a nucleic acid sequence comprising SEQ ID NO:39. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:33, a nucleic acid sequence comprising SEQ ID NO:35, and a nucleic acid sequence comprising SEQ ID NO:39. In some embodiments, the present invention provides a composition comprising a nucleic acid preparation useful for reprogramming a non-hepatocyte cell to an induced hepatocyte, wherein the nucleic acid preparation comprises nucleic acid molecules comprising a nucleic acid sequence comprising SEQ ID NO:35, a nucleic acid sequence comprising SEQ ID NO:37, and a nucleic acid sequence comprising SEQ ID NO:39. In various embodiments, the nucleic acids useful for reprogramming a non-hepatocyte cell to an induced hepatocyte are DNA molecules or mRNA molecules, preferably purified DNA molecules or purified mRNA molecules, encoding the reprogramming factors.

### Characterization and Identification of Induced Hepatocytes

Induced hepatocytes of the present invention can be characterized and identified according to a number of criteria. These criteria include, but are not limited to, the detection or quantitation of expressed hepatocyte cell markers, enzymatic activity, hepatocyte function, characterization of hepatocyte morphological features, etc. In some aspects, non-hepatocyte cells to be reprogrammed to induced hepatocytes may contain reporter genes comprising hepatocyte-specific transcriptional control elements, such as hepatocyte-specific gene promoters, which can be used for identification of an induced hepatocyte.

In certain aspects, induced hepatocytes of the present invention have morphological features characteristic of hepatocytes, such as that observed in primary hepatocytes obtained from organ sources. Morphological features characteristic of hepatocytes are readily appreciated by those skilled in the art, and may include any or all of the following: a polygonal cell shape, a bi-nucleated phenotype, the presence of rough endoplasmic reticulum for synthesis of secreted protein, relatively abundant or extensive vacuoles, lipid droplets, the presence of Golgi-endoplasmic reticulum lysosome complex for intracellular protein sorting, the presence of peroxisomes and glycogen storage granules, relatively abundant mitochondria, and the ability to form tight intercellular junctions resulting in creation of bile canalicular spaces. One or more of these morphological features present in a single cell are consistent with the cell being one of hepatocyte lineage. The induced hepatocytes of the present invention can have any one or more of these morphological features characteristic of hepatocytes and can thus be characterized or identified according to any one or more of these morphological features.

In other aspects, induced hepatocytes of the present invention can be characterized or identified according to the expression or induction of various phenotypic markers characteristic of hepatocytes. Non-limiting examples of phenotypic markers characteristic of hepatocytes and useful to identify induced hepatocytes include, for example, albumin, α-fetoprotein, alpha-1-anti-trypsin, cytokeratin 18, and DLK1. Other examples of phenotypic markers characteristic of hepatocytes include tryptophan 2,3-dioxygenase (Tdo2), transferrin, E-cadherin, tight junction protein (Tjp1), asialoglycoprotein receptor, apoE, arginase 1, apoAI, apoAII, apoB, apoCIII, apoCII, alsolase B, alcohol dehydrogenase 1, catalase, glucose-6-phosphatase, γ-glutamyl transpeptidase, production of urea, synthesis of triglyceride, cytochrome p450 activity, etc. The induced hepatocytes of the present invention can have any one or more of these phenotypic markers characteristic of hepatocytes and can thus be characterized or identified based on the expression or activity of any one or more of these phenotypic markers.

In some embodiments, the expression of certain markers is determined by detecting the presence or activity of a marker or, in some instances, the absence of a marker. The expression of markers characteristic of hepatocytes can be determined by detecting the presence of a marker within, on the surface of, or secreted by a cell or cells of the cell culture or cell population. The detection can be quantitative or qualitative. Protein expression can be determined by any suitable immunological means, such as, for example, flow cytometry, immunohistochemistry, Western blot analysis, enzyme-linked immunoassay (ELISA), etc.

Gene expression of hepatocyte markers can be determined by PCR (including quantitative PCR, real time PCR, etc.), northern blot analysis, in situ hybridization, etc. Methods for performing such techniques are well known and available to one of skill in the art. Other methods, which are known in the art, can also be used to quantitate hepatocyte marker gene expression. For example, the expression of a marker gene product can be detected by using antibodies specific to the marker gene product of interest. Nucleic acid hybridization or amplification techniques can be used to detect the presence, absence, and/or level of expression of one or more markers of hepatocytes. Nucleic acid hybridization techniques include, for example, Northern blots, slot blots, RNase protection, in situ hybridization, and the like. Nucleic acid amplification techniques include PCR, real time PCR, quantitative PCR, etc. Techniques for the detection and quantitation of specific nucleic acids are well-known to those skilled in the art and are described in, for example, Ausubel, etc. (See, e.g., International Application Publication No. WO 2007/127454)

In certain aspects, the expression of marker genes characteristic of hepatocytes as well as the lack of significant expression of marker genes characteristic of non-hepatocyte cells (e.g., fibroblasts, keratinocytes, etc.) is determined.

### Compositions Comprising Induced Hepatocytes

The present invention provides cell compositions comprising induced hepatocytes produced by the methods of the present invention. In some embodiments, various compositions of induced hepatocytes, such as cell cultures or cell populations, which are substantially free of cells other than hepatocytes, are provided by the present invention. In addition, compositions, such as cell cultures or cell populations that are enriched, isolated, or purified for induced hepatocytes are provided.

Compositions of induced hepatocytes provided by the present invention may comprise a homogeneous population of induced hepatocytes. In some aspects, the present invention provides a composition comprising induced hepatocytes, wherein the composition comprises a homogeneous population of induced hepatocytes. In some embodiments, the present invention provides a composition of induced hepatocytes, wherein the percentage of cells within the composition comprises about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% induced hepatocytes. In some embodiments, the percentage of cells in a composition comprising induced hepatocytes is 100% induced hepatocytes.

### Methods of Using Induced Hepatocytes

The present invention provides induced hepatocytes or populations of induced hepatocytes useful in various research and therapeutic applications. Such applications include transplantation or implantation of the induced hepatocytes in vivo; screening cytotoxic compounds, carcinogens, mutagens, growth/regulatory factors, pharmaceutical compounds, etc., in vitro; elucidating the mechanism of various liver diseases and liver infections; production of biologically active products, etc. For example, the induced hepatocytes of the present invention can be used to further studies in cell and tissue differentiation. The induced hepatocytes described herein can also be used in toxicity assays for testing new drug and therapeutic candidates. Moreover, the induced hepatocytes of the present invention can be used for regenerative medicine and therapeutic use.

Induced hepatocytes of the present invention can be used for screening various factors (such as solvents, therapeutics, peptides, and polynucleotides) or environmental conditions (such as culture conditions or manipulations), which may affect various characteristics of induced hepatocytes, as provided herein.

In some aspects, various screening applications of the present invention relate to the testing of pharmaceutical compounds or agents in drug research. (See, e.g., Castell et al., In vitro Methods in Pharmaceutical Research, Academic Press, 1997; and U.S. Pat. No. 5,030,015.) In certain aspects, the induced hepatocytes of the present invention are useful for standard drug screening and toxicity assays, as have been performed previously on hepatocyte cell lines or primary hepatocytes. Assessment of the activity of candidate pharmaceutical compounds or agents generally involves combining the induced hepatocytes with the candidate compound, determining any change in the morphology, marker phenotype, or metabolic activity of the cells that is attributable to the compound or agent (compared with untreated cells or cells treated with an inert compound or an inert agent), and then correlating the effect of the compound or agent with the observed change. The screening may be done because the compound or agent is designed to have a pharmacological effect on hepatocytes, or because the compound or agent may be designed to have effects elsewhere (e.g., designed to have effects on non-hepatocyte cells) and thus may have unintended hepatocyte side effects. Two or more compounds or agents (e.g., two or more drugs) can be tested in combination (by combining with the induced hepatocytes either simultaneously or sequentially), to detect possible drug-drug interaction effects.

In some aspects, the induced hepatocytes of the present invention are useful to screen a compound for potential cytotoxicity or hepatotoxicity. (See Castell et al., (1997) supra.)

Cytotoxicity or hepatotoxicity can be determined by the effect of a compound on cell viability, survival, morphology, and leakage of enzymes and other factors into the culture medium, or by determining the effects of the compound on hepatocyte function (such as, for example, effects on gluconeogenesis, ureagenesis, plasma protein synthesis, and the like).

Other methods to evaluate cytotoxicity or hepatotoxicity include determination of the effect of a compound on the synthesis and secretion of albumin, cholesterol, and lipoproteins; transport of conjugated bile acids and bilirubin; ureagenesis; cytochrome p450 levels and activity; glutathione levels; release of a-glutathione s-transferase; ATP, ADP, and AMP metabolism; intracellular K+ and Ca2+ concentrations; the release of nuclear matrix proteins or oligonucleosomes; and induction of apoptosis. Effects of a compound on DNA synthesis or structure can be determined by measuring DNA synthesis or repair.

The induced hepatocyte cells of the present invention can also be used as a tool for drug testing and development process. For example, the cells can be used to assess changes in gene expression patterns caused by a drug candidate (e.g., a therapeutic drug candidate) being considered for development. The changes in gene expression pattern from potential drugs can be compared with those caused by control drugs known to affect the liver. This allows one to screen compounds and drugs for their effects on the liver earlier in the drug development process without using animals, thereby saving time and money. In some embodiments, the induced hepatocytes of the present invention are used in a high throughput drug screening, such as in the manner described in U.S. Pat. No. 7,282,366.

The induced hepatocytes of the present invention can also be used to assess toxicity of various compounds or compositions of interest, e.g., chemical, pharmaceutical, cosmetic, biocidal or biological compounds, food additives or compositions, or other biological agents. The use of induced hepatocyte may be preferred in such assays of toxicity, as the induced hepatocytes more closely resemble the cell types present in the liver of an organism. For example, a particular compound or composition is considered toxic or likely toxic, if it shows a detrimental effect on the viability of cells or on one or more aspect of cellular metabolism or function. The viability of cells in vitro may be measured using techniques known in the art, including colorimetric assays, such as the MTT (or MTT derivative) assays or LDH leakage assays, or using fluorescence-based assays, such as, e.g., the Live/Dead assay, CyQuant cell proliferation assay, or assays of apoptosis. Other useful assays include those that measure particular aspects of cellular metabolism, protein expression and secretion, function, etc., as described herein.

In some embodiments, the induced hepatocytes of the present invention can be used as a model of various human diseases and disorders. In many aspects, the induced hepatocytes of the present invention provide a cell-based in vitro model of human disease, and can be used for various experimental and research purposes. In certain aspects of this use, non-hepatocyte cells for use in these methods are obtained from an individual having a disease or disorder. For example, some human disorders, such as certain hepatic disorders, have a monogeneic or a polygeneic basis, and non-hepatocyte cells obtained from individuals having such disorders can be reprogrammed to induced hepatocytes using methods provided herein. In other aspects of this use, non-hepatocyte cells for use in these methods can be obtained from various individuals, allowing for the production of induced hepatocytes of broad genetic diversity, thus allowing for modeling human disease in cells obtained from a large number and variety of donors. Such use provides an advantage over current methods for modeling human disease in which the cell source is of limited diversity (e.g., primary hepatocytes, cell lines, hepatocytes derived from pluripotent cells).

An advantage to such a therapeutic approach is not having the requirement of obtaining a liver biopsy. The use of induced pluripotent stem cells derived from patients and differentiated into disease-relevant cell types has been described, and the present invention contemplates the use of induced hepatocytes provided herein for such disease modeling applications. (See, e.g., Grskovic et al., (2011) Nature Reviews Drug Discovery 10:915-929; Rashid et al., (2010) J Clinical Investigation 120:3127-3136.)

In other embodiments, the induced hepatocytes of the present invention can be used as models for hepatitis and other liver disease studies. Such models can be useful in the development of therapeutics, such as anti-viral therapeutics, etc. The induced hepatocytes of the present invention provide a constant source of hepatocytes for research use. Hepatitis virus-infected induced hepatocytes can be cultured in vitro and researches can study the progression of the disease and virus life-cycle. Virus-infected induced hepatocytes can also be used to screen for pharmaceutical agents and compounds that, for example, halt the virus life-cycle, kill the virus, or eradicate the virus from the induced hepatocytes; such screenings can also provide information on cytotoxicity of various pharmaceutical agents and compounds.

In other embodiments, the induced hepatocytes of the present invention can be used for genetic correction by generating induced hepatocytes for autologous cell-based therapies. (See, e.g., Yusa et al., (2011) Nature 478:391-394

Induced hepatocytes derived from a population of non-hepatocyte cells provided by the present invention can find advantageous use in a variety of research and clinical applications, including, e.g., absorption, distribution, metabolism, excretion, and toxicity studies and therapeutic liver regeneration. The present invention provides populations of induced hepatocytes that can be used to treat various degenerative liver diseases or inherited or acquired deficiencies of liver function. Because the liver controls the clearance and metabolism of drugs (e.g., small molecule drugs), the induced hepatocytes provided by the present invention can be used to evaluate and/or model the in vivo effects of candidate drugs and therapeutics on liver cells.

In other embodiments, the induced hepatocytes of the present invention can be used for biomarker identification. Such uses include the use of known biomarkers for predicting hepatotoxicity or efficacy, identification of novel biomarkers for predicting hepatotoxicity or efficacy, and the application of such novel biomarkers in various clinical studies for identifying those patients or groups of patients (e.g., subpopulations of individuals) who will respond, or will likely respond, to various treatments by identification and correlation of biomarker expression by the induced hepatocytes, wherein one or more biomarkers can identify whether an individual will respond positively or negatively to a treatment. In various aspects of this use, the induced hepatocytes are produced from non-hepatocyte cells derived from the patient for which treatment is contemplated.

### Cell-Based Therapies

The induced hepatocytes or population of induced hepatocytes of the present invention can be used in various methods of treatment, such as, for example, the treatment of patients having damaged or dysfunctional hepatic tissue or liver disease.

The induced hepatocytes or population of induced hepatocytes of the present invention can also be used in the manufacture of a medicament for use in the treatment of damaged or dysfunctional hepatic tissue. An individual with damaged or dysfunctional hepatic tissue or liver disease may have hepatitis, liver fibrosis, cirrhosis, hepatocellular carcinoma, non-alcohol fatty liver disease, drug-induced liver injury, alcoholic liver disease, autoimmune liver disease, or an inherited metabolic disorder of the liver, such as α1-antitrypsin deficiency, glycogen storage disease, familial hypercholesterolemia, etc. The present invention contemplates herein the use of induced hepatocytes for treatment of these hepatic disorders.

The use of induced hepatocytes for therapeutic liver regeneration offers a vast improvement over current cell therapy procedures that utilize donor livers (i.e., liver transplant) or cells obtained from donor livers for the treatment of liver disease. The present invention provides a source of induced hepatocytes that can be developed for such treatments. One advantage of using induced hepatocytes produced from non-hepatocytes cells of the obtained from the patient is the elimination immune-mediated rejection by the recipient.

In some embodiments, the present invention provides methods for the treatment of damaged or dysfunctional hepatic tissue or liver disease, the method comprising administering to a patient in need thereof a population of induced hepatocytes obtained by the methods described herein, thereby providing treatment of damaged or dysfunctional hepatic tissue or liver disease in the patient. The population of induced hepatocytes may be administered to the patient in need thereof by transplantation, infusion, or otherwise administration into the liver of the patient.

### EXAMPLES

The following are examples of methods and compositions of the invention. It is understood that various other embodiments may be practiced, given the general description provided above.

### Methods and Materials

### Cell Culture

BJ fibroblasts (human primary foreskin fibroblasts, Stemgent, Cambridge, Mass.) and MRC-5 fibroblasts (human fetal lung fibroblasts, ATCC Cat. No. CCL-171) were cultured in DMEM/F12+Glutamax media (Invitrogen, Carlsbad, Calif.) containing 10% HyClone FBS (Thermo Scientific, Waltham, Mass.), 1% Insulin-Transferrin-Selenium (Invitrogen), 1% MEM Non-Essential Amino Acids (Invitrogen), and 5 mM HEPES buffer. After a two day expansion, fibroblasts were dissociated with 0.5% Trypsin-EDTA (Invitrogen) and plated at 1000 cells/cm² on Collagen-I coated tissue culture plates (BD Biosciences, San Jose, Calif.) for subsequent reprogramming experiments. For reprogramming experiments, cell culture media was supplemented with 20 ng/ml human hepatocyte growth factor (HGF), 20 ng/ml epidermal growth factor (EGF), 20 ng/ml fibroblast growth factor 2 (FGF2) (Peprotech, Rocky Hill, N.J.), 200 ng/mL B18R (eBioscience, San Diego, Calif.), and 0.1 μM dexamethasone (Sigma). All cell culture was performed in antibiotic-free culture media.

### DNA Template Construction for In Vitro Transcription (IVT)

DNA template construction for use in in vitro transcription reactions was performed using a modification of PCR amplification and DNA ligation methods previously described. (See Warren et al. (2010) Cell Stem Cell 7:618-630.) Oligonucleotides, including primers, splints, and UTRs, were synthesized at the Genentech oligonucleotide synthesis core facility. Forward ORF primers were 3′ phosphorylated, and the 3′ UTR was 5′ phosphorylated upon oligonucleotide synthesis to maximize ligation efficiency.

Open reading frame (ORF) PCR amplifications of DNA encoding Foxa1, Foxa2, Foxa3, Hnf4α, Hnf1α, and Gata4 were templated from DNA plasmids containing each of the respective human ORFs (Origene, Rockville, Md.). Sequences of oligonucleotide primers used for each ORF PCR amplification are shown in Table 1 below.

TABLE 1

| | ORF Forward Primer | ORF Reverse Primer |
|---|---|---|
| FOXA1 | TTAGGAACTGTGAAGATGGAAGG | CTAGGAAGTGTTTAGGACGGGTCT |
| | (SEQ ID NO: 1) | (SEQ ID NO: 2) |
| FOXA2 | CACTCGGCTTCCAGTATGCT | TTAAGAGGAGTTCATAATGGGC |
| | (SEQ ID NO: 3) | (SEQ ID NO: 4) |
| FOXA3 | CTGGGCTCAGTGAAGATGGA | CTAGGATGCATTAAGCAAAGAGC |
| | (SEQ ID NO: 5) | (SEQ ID NO: 6) |
| HNF4A | CGACTCTCCAAAACCCTCGT | CTAGATAACTTCCTGCTTGGTGA |
| | (SEQ ID NO: 7) | (SEQ ID NO: 8) |
| HNF1A | GTTTCTAAACTGAGCCAGCTGC | TTACTGGGAGGAAGAGGCC |
| | (SEQ ID NO: 9) | (SEQ ID NO: 10) |
| GATA4 | TATCAGAGCTTGGCCATGG | TTACGCAGTGATTATGTCCCC |
| | (SEQ ID NO: 11) | (SEQ ID NO: 12) |
| NLS- | CCTAAGAAGAAGCGTAAGGAGAGCGACGAGAGCG | CTATTCTTCACCGGCATCTG |
| GFP | (SEQ ID NO: 13) | (SEQ ID NO: 14) |
| CEBPA | GAGTCGGCCGACTTCTACG | TCACGCGCAGTTGCCCAT |
| | (SEQ ID NO: 45) | (SEQ ID NO: 46) |
| GATA6 | GCCTTGACTGACGGCGG | TCAGGCCAGGGCCAGGG |
| | (SEQ ID NO: 47) | (SEQ ID NO: 48) |
| HHEX | CAGTACCCGCACCCCG | TCATCCAGCATTAAAATAGCTTTTATC |
| | (SEQ ID NO: 49) | (SEQ ID NO: 50) |
| HNF1B | GTGTCCAAGCTCACGTCG | CCAGGCTTGTAGAGGACACTG |
| | (SEQ ID NO: 51) | (SEQ ID NO: 52) |
| HNF6A | AACGCGCAGCTGACCAT | TCATGCTTTGGTACAAGTGCT |
| | (SEQ ID NO: 53) | (SEQ ID NO: 54) |

Following ORF PCR amplifications, oligonucleotides containing untranslated regions (UTRs) were then joined to the top strand of the ORF PCR amplification products by DNA ligase (ampligase), mediated by annealing the amplification products to splint oligonucleotides which bring the desired single-strand DNA ends together. Sequences of splint oligonucleotides used for DNA ligations are shown in Table 2 below.

TABLE 2

| | 5′ Splint Oligonucleotide | 3′ Splint Oligonucleotide |
|---|---|---|
| FOXA1 | CCTTCCATCTTCACAGTTCCTAACATGGTGGCTCTTATATTTCTTCTT | CCCGCAGAAGGCAGCCTAGGAAGTGTTTAGGACG |
| | (SEQ ID NO: 15) | (SEQ ID NO: 16) |
| FOXA2 | AGCATACTGGAAGCCGAGTGCATGGTGGCTCTTATATTTCTTCTT | CCCGCAGAAGGCAGCTTAAGAGGAGTTCATAATGGGC |
| | (SEQ ID NO: 17) | (SEQ ID NO: 18) |
| FOXA3 | CCATCTTCACTGAGCCCAGCATGGTGGCTCTTATATTTCTTCTT | CCCGCAGAAGGCAGCCTAGGATGCATTAAGCAAAGAG |
| | (SEQ ID NO: 19) | (SEQ ID NO: 20) |
| HNF4A | CGAGGGTTTTGGAGAGTCGCATGGTGGCTCTTATATTTCTTCTT | CCCGCAGAAGGCAGCCTAGATAACTTCCTGCTTGG |
| | (SEQ ID NO: 21) | (SEQ ID NO: 22) |
| HNF1A | GCAGCTGGCTCAGTTTAGAAACCATGGTGGCTCTTATATTTCTTCTT | CCCGCAGAAGGCAGCTTACTGGGAGGAAGAGGC |
| | (SEQ ID NO: 23) | (SEQ ID NO: 24) |
| GATA4 | CCATGGCCAAGCTCTGATACATGGTGGCTCTTATATTTCTTCTT | CCCGCAGAAGGCAGCTTACGCAGTGATTATGTCCCC |
| | (SEQ ID NO: 25) | (SEQ ID NO: 26) |
| NLS- | CTCTCCTTACGCTTCTTCTTAGGCATGGTGGCTCTTATATTTCTTCTT | CCCGCAGAAGGCAGCCTATTCTTCACCGGCATCTG |
| GFP | (SEQ ID NO: 27) | (SEQ ID NO: 28) |
| CEBPA | CGTAGAAGTCGGCCGACTCCATGGTGGCTCTTATATTTCTTCTT | CCCGCAGAAGGCAGCTCACGCGCAGTTGCCCAT |
| | (SEQ ID NO: 55) | (SEQ ID NO: 56) |
| GATA6 | CCGCCGTCAGTCAAGGCCATGGTGGCTCTTATATTTCTTCTT | CCCGCAGAAGGCAGCTCAGGCCAGGGCCAGGG |
| | (SEQ ID NO: 57) | (SEQ ID NO: 58) |
| HHEX | CGGGCCCCACGCCCATGACCATGGTGGCTCTTATATTTCTTCTT | CCCGCAGAAGGCAGCTCATCCAGCATTAAAATAGC |
| | (SEQ ID NO: 59) | (SEQ ID NO: 60) |
| HNF1B | CGACGTGAGCTTGGACACCATGGTGGCTCTTATATTTCTTCTT | CCCGCAGAAGGCAGCCCAGGCTTGTAGAGGACACT |
| | (SEQ ID NO: 61) | (SEQ ID NO: 62) |
| HNF6A | ATGGTCAGCTGCGCGTTCATGGTGGCTCTTATATTTCTTCTT | CCCGCAGAAGGCAGCTCATGCTTTGGTACAAGTGC |
| | (SEQ ID NO: 63) | (SEQ ID NO: 64) |

The sequences of the 5′-UTR and 3′-UTR used were as follows: (See Warrant et al., (2010) Cell Stem Cell 7:618-630.)

5′-UTR:
(SEQ ID NO: 29)
TTGGACCCTCGTACAGAAGCTAATACGACTCACTATAGGGAAATAAGAGA
GAAAAGAAGAGTAAGAAGAAATATAAGAGCCACCATG;
3′-UTR:
(SEQ ID NO: 30)
GCTGCCTTCTGCGGGGCTTGCCTTCTGGCCATGCCCTTCTTCTCTCCCTT
GCACCTGTACCTCTTGGTCTTTGAATAAAGCCTGAGTAGGAAGTGAGGGT
CTAGAACTAGTGTCGACGC.

The ligation products obtained using the above methods were then PCR amplified using forward and reverse tailing oligonucleotide primers, which amplify the above ligation product and simultaneously add the polyA+ tail. Forward and reverse tailing primer sequences used are shown in Table 3 below.

TABLE 3

| Forward Tailing Primer | Reverse Tailing Primer |
|---|---|
| TTGGACCCTCGTACAGAAGC | T₁₂₀ CTTCCTACTCAGGCTTTATTCAAA |
| (SEQ ID NO: 31) | (SEQ ID NO: 32) |

ORF PCR amplifications for NLS-GFP (green fluorescent protein (GFP) engineered to contain a nuclear localization sequence (NLS)) were templated from pturboGFP plasmid (Evrogen through Axxora, Richmond, Va.). A nuclear localization sequence for NLS-GFP was added to the N-terminal end of GFP using a modified forward primer, as shown in Table 2 above, SEQ ID NO:27.

Intermediate ORF PCR amplification products and ligation products were purified using QIAquick PCR purification columns (Qiagen, Valencia, Calif.). Final template PCR amplification products were separated on 1.2% Agarose SYBR E-Gels (Invitrogen). Bands of the correct length were excised from the gels and purified sequentially using QIAquick Gel Extraction and QIAquick PCR purification columns (Qiagen). Isolated and purified templates were then used for synthesis of modified mRNA as described below.

mRNA Synthesis

mRNA was synthesized by in vitro transcription (IVT) using a MEGAscript T7 kit (Ambion, Austin, Tex.), with 1.5 μg DNA template used in each 40 μl reaction. In order to synthesize mRNA molecules having reduced immunogenicity and increased stability, a modified ribonucleoside mixture was used in the IVT reactions, as previously described (Warren et al. (2010) supra). Final concentrations of ribonucleosides in the IVT reaction mixtures were as follows: 7.5 mM ATP, 7.5 mM pseudo-UTP, 7.5 mM methyl-CTP, 1.5 mM GTP, and 6 mM ARCA. Pseudo-UTP, methyl-CTP, and ARCA were obtained from TriLink BioTechnologies (San Diego, Calif.); ATP and GTP were obtained from Affymetrix (Santa Clara, Calif.).

In vitro transcription reactions were allowed to proceed either for 14-16 hrs at 30° C. or for 3-6 hrs at 37° C. and subsequently treated with DNase as described by the manufacturer. The resulting mRNA thus contained a polyA+ tail of at least 120 As (SEQ ID NO: 66) (incorporated by using a T₁₂₀-heeled primer; see Table 3, SEQ ID NO:32; “T₁₂₀” disclosed as SEQ ID NO: 65)) and a 5′ anti-reverse cap analog (ARCA) to assist in recruiting the ribosomal initiation complex. Both a polyA+ tail of at least 120 As (SEQ ID NO: 66) and ARCA protect the resulting mRNA from endonucleases and degradation following transfection into cells. RNA was purified, treated with phosphatase to remove residual 5′ phosphates, and then re-purified. A MEGAclear Kit (Ambion)w was used for all IVT mRNA purifications. RNA length and purity was assessed using an RNA 6000 Pico Kit with an Agilent 2100 Bioanalyzer (Agilent Technologies, Santa Clara, Calif.). RNA concentration was determined by Nanodrop (Thermo Scientific) and adjusted to a stock concentration of 200 ng/μl by addition of Nuclease-free water (Ambion). A GFP-encoding mRNA was used for non-nuclear GFP transfection (Maxcyte, Gaithersburg, Md.).

### RNA Transfection

TransIT-mRNA (Minis Bio, Madison, Wis.) cationic lipid reagent was used for transfection of mRNA into cells. Before transfection, mRNA was diluted 20-fold in Opti-MEM Reduced Serum Media (Invitrogen), and BOOST reagent was added at 2 μl per microgram of RNA, after which TransIT-mRNA reagent was added at a ratio of 2 μl TransIT-mRNA reagent per microgram of RNA. The resulting RNA-lipid complexes were incubated at room temperature for 3 minutes and then delivered to the cells. Cell culture media was changed immediately prior to transfection of the mRNA into cells.

### Immunostaining

Cells were fixed in 4% formaldehyde for 15 minutes and washed 3 times for 5 minutes with PBS. The cells were then blocked for 1 hour at room temperature in 5% goat serum (Cell Signaling, Dansvers, Mass.) or 5% donkey serum (Sigma, St. Louis, Mo.) containing 0.3% Triton X-100 (Sigma). The cells were incubated with primary antibodies for 2 hours at room temperature and with secondary antibodies for 1 hour at room temperature in 1% BSA (Sigma) and 0.3% Triton X-100. Cells were washed 3 times for 5 minutes with PBS after primary antibody incubation.

The antibodies used for immunostaining were as follows: Foxa1 (Abcam, Cambridge, Mass.), Foxa2 (Cell Signaling), and Foxa3 (Santa Cruz Biotech, Santa Cruz, Calif.) primary antibodies were used at 1:50 dilutions; Hnf4α (Cell Signaling), Hnf1α (BD Biosciences), and albumin (Abnova, Walnut, Calif.) primary antibodies were used at 1:100 dilutions; Gata4 (BD Biosciences) and α-fetoprotein (AFP) (Sigma) primary antibodies were used at 1:200 dilutions. Anti-mouse IgG, anti-rabbit IgG, and anti-goat IgG Alexa Fluor 488 and 555 secondary antibodies (Invitrogen) were used at 1:1000 dilutions. HCS LipidTOX Neutral Lipids Stain (Invitrogen) was used for lipid droplet staining as directed by the manufacturer. Hoechst 33342 (Invitrogen) was used at 1 μg/ml for nuclear staining. Images were acquired using an IX81 Inverted microscope (Olympus, Center Valley, Pa.).

qPCR

Quantitative PCR (qPCR) was performed as follows. A Cells-to-Ct kit (Ambion) was used for RNA extraction according to the manufacturer's instructions, and 22.5 μl of this was carried over to 50 μl qPCR reactions. For the experiments described in Example 8, RNA was isolated directly from cell culture wells using a miRNeasy Mini kit (Qiagen). 1 μg RNA from each sample was used in 50 μl qPCR reactions from a Cells-to-Ct kit (Ambion). Before qPCR reactions, RNA samples were treated with DNase according to the respective manufacturers' instructions. For qPCR, 4 μl of each qPCR was used in 20 μl reactions with Taqman Universal Master Mix, no UNG (Applied Biosystems, Foster City, Calif.). Primer/probes used were 20× Taqman Gene Expression Assays (Applied Biosystems) as follows: ABCB1 (Hs01067802_ml), ABCB11 (Hs00184824_ml), ABCG2 (Hs01053790_ml), AFM (Hs00265717_ml), AFP (α-fetoprotein; Hs01040601_ml), ALB (albumin; Hs00910225_ml), ALB (albumin; Hs00609411_m), B2M (Hs00984230_ml), CD106 (Hs01003372_ml), CD13 (Hs00174265_ml), CD133 (Hs01009250_ml), CD26 (Hs00175210_ml), CD29 (Hs00559595_ml), CD36 (Hs00169627_ml), CD44 (Hs01075861_ml), CD73 (Hs00159686_ml), CD9 (Hs00233521_ml), CD90 (Hs00174816_ml), CDH2 (Hs00983056_ml), CXCR4 (Hs00237052_ml), CYP1A2 (Hs00167927_ml), CYP2C19 (Hs00426380_ml), CYP2C8 (Hs02383390_sl), CYP2C9 (Hs00426397_ml), CYP2D6 (Hs00164385_ml), CYP2E1 (Hs00559368_ml), CYP3A4 (Hs00256159_ml), CYP3A7 (Hs00426361_ml), CYP7A1 (Hs00167982_ml), DES (Hs00157258_ml), DLK1 (Hs00171584_ml), EGFR (Hs00193306_ml), FABP1 (Hs00155026_ml), FGFR4 (Hs00242558_ml), FOXA1 (Hs04187555_ml), FOXA2 (Hs00232764_ml), FOXA3 (Hs00270130_ml), GAPDH (Hs03929097_gl), GATA4 (Hs00171403_ml), GPC3 (Hs00170471_ml), GSTA1 (Hs00275575_ml), HNF1A (Hs00167041_ml), HNF4A (Hs00230853_ml), ICAM1 (Hs00164932_ml), IGF2 (Hs01005970_ml), IL6R (Hs00794121_ml), KRT15 (Hs00267035_ml), KRT18 (cytokeratin-18, ck18; Hs01941416_gl), KRT19 (Hs00761767_sl), KRT8 (Hs01630795_sl), MET (Hs00179845_ml), NNMT (Hs00196287_ml), OSMR (Hs00384276_ml), RPL19 (Hs02338565_gH), SERPINA1 (alpha1-anti-trypsin; Hs01097800_ml), SERPINA3 (Hs00153674_ml), SLCO1B3 (Hs00251986_ml), SLCO2B1 (Hs00200670_ml), TTR (Hs00174914_ml), and UGT2B4 (Hs02383831_sl).

RPL19 (Hs02338565_gH) was used for endogenous control assays. The ViiA 7 Real Time PCR System (Applied Biosystems) was used to perform and analyze qPCRs. If gene expression was not detectable in a sample, an artificial cycle threshold of 41 was applied to allow for comparison.

### Reprogramming Experiments

Reprogramming experiments were generally performed as follows. Initial reprogramming experiments used mRNA pools containing six different mRNAs encoding the following six transcription factors: FOXA1, FOXA2, FOXA3, HNF4A, HNF1A, and GATA4. This six transcription factor pool containing mRNAs encoding FOXA1, FOXA2, FOXA3, HNF4A, HNF1A, and GATA4 is herein referred to as 6TF. The 6TF mRNA mixture used in these studies contained the mRNA molecules at a molar ratio of 1:1:1:1:1:1. A total dose of approximately 1,200 ng mRNA per 6-well plate was transfected into the cultured cells once daily at the same time each day for the indicated number of days, unless otherwise indicated. (See Table 4 below.)

In experiments using various combinations of transcription factor mixtures, the final amount of each individual mRNA encoding a transcription factor (but not the final total mRNA content) remained constant in Examples 11 and 14. The final total mRNA content (but not the final amount of each individual factor) remained constant in Examples 12 and 13. The exact transcription factor compositions as well as the amounts of mRNA used for the daily transfections of the mRNA pools used for reductive and additive experiments are shown in the Examples below. (See Tables 5, 6, 7, and 8 below.)

### Example 1

### Protein Expression Correlates with Dose of Transfected of mRNA

Initial experiments were performed to examine the effect of various doses of transfected mRNA on respective protein expression and localization following transfection into human fibroblasts. Various amounts of IVT mRNA encoding green fluorescent protein (GFP) or nuclear localization-GFP (NLS-GFP) (approximately 80 ng, 200 ng, 500 ng, and 1,000 ng, prepared as described above) were transfected into BJ fibroblasts grown in 24-well tissue culture plates, as described above. Cells were transfected one time with the indicated amounts of RNA and cultured for an additional 24 hours. The expression of GFP was determined using methods described above.

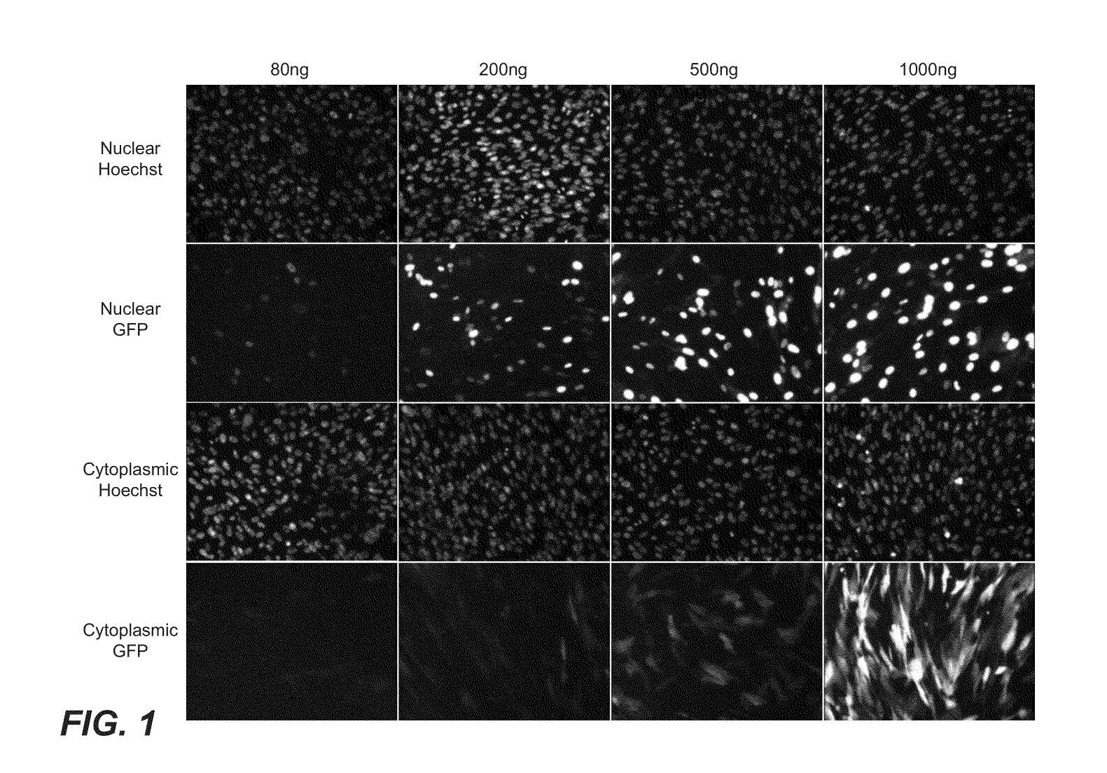

As shown in FIG. 1, expression of GFP in human fibroblasts transfected with mRNA encoding GFP (labeled as Cytoplasmic GFP in FIG. 1) or GFP containing a nuclear localization sequence (NLS-GFP, labeled as Nuclear GFP in FIG. 1) showed a dose-response of protein expression that correlated with the amount of mRNA transfected. Further, the addition of a nuclear localization sequence to the N-terminus of GFP mRNA resulted in proper nuclear localization of the expressed GFP protein. These results indicated that the degree of expression of transcription factors encoded by individual mRNAs can be adjusted in a dose-dependent fashion.

### Example 2

### Transcription factor Expression Following Daily Transfection of Fibroblasts with Six Transcription Factor mRNA Mixture

To examine the extent to which transcription factor expression is maintained following mRNA transfection into human fibroblasts, the following studies were performed. An mRNA mixture was prepared by pooling 6 transcription factor (6TF) mRNAs (encoding FOXA1, FOXA2, FOXA3, HNF4A, HNF1A, and GATA4) transcribed in vitro as described above, at a molar ratio of 1:1:1:1:1:1. This 6TF mRNA mixture was transfected (containing approximately 1,200 ng total mRNA/transfection/6-well culture plate) once daily into BJ fibroblasts cultured in 6-well culture plates for 9 days, as described above. (See Table 4 below, listing the amounts of each mRNA within the 6TF mRNA mixture.) After 9 days, the expression of each transcription factor was measured by qPCR and compared to the expression of each transcription factor in non-transfected BJ fibroblasts (e.g., lipid-only control transfected cells), using methods described above.

TABLE 4

| Transcription Factor | ng mRNA |
|---|---|
| FOXA1 | 200 |
| FOXA2 | 197 |
| FOXA3 | 156 |
| HNF4A | 201 |
| HNF1A | 257 |
| GATA4 | 189 |
| Total (ng) transfected mRNA/well | 1,200 |

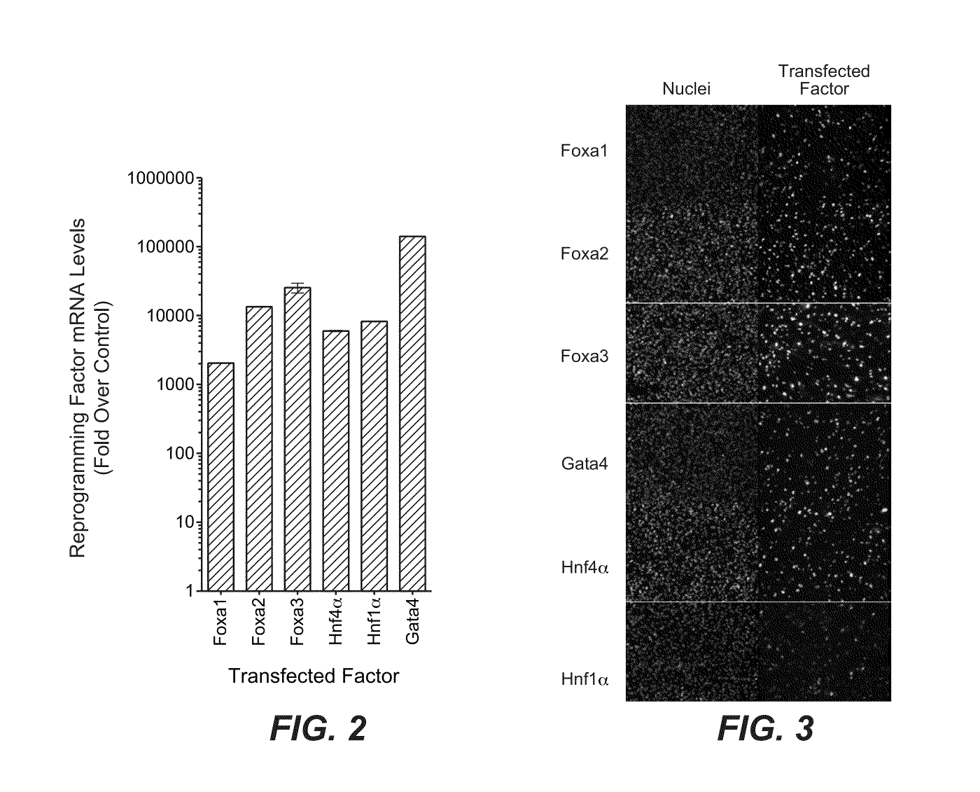

As shown in FIG. 2, elevated transcription factor mRNA levels encoding the various reprogramming factors were maintained in cells transfected once-daily with 6TF mRNA mixture containing equimolar amounts of each transcription factor mRNA. These results indicated that the 6TF mRNA mixture (as described above) was consistently delivered to the cultured human BJ fibroblasts. No loss of cell viability and no immunogenicity against the sustained mRNA delivery and exposure were observed over the 9-day course of these experiments (data not shown).

### Example 3

### Transcription Factors Localize to the Nucleus in Human Fibroblasts Transfected with Six Transcription Factor mRNA Mixture

In order to examine the cellular localization of the transcription factors within cells transfected with 6TF mRNA mixture, the following studies were performed. BJ fibroblasts were transfected once with 6TF mRNA mixture as described above. After 24 hours, the cellular localization and qualitative protein expression of each transcription factor was determined by immunostaining, using methods described above. FIG. 3 shows immunostaining of GATA4, HNF1A, HNF4A, FOXA3, FOXA2, and FOXA1 following transfection of 6TF mRNA mixture into BJ fibroblasts. As shown in FIG. 3, GATA4, HNF1A, HNF4A, FOXA3, FOXA2, and FOXA1 protein expression was detected and localized to the nuclei of transfected cells. Similar localization results were obtained in cells transfected once-daily for 5 days. These results indicated that the transcription factors were expressed and correctly localized to the nuclei of the transfected cells.

### Example 4

### Induction of Albumin and α-Fetoprotein in Human Fibroblasts Transfected with Six Transcription Factor mRNA Mixture

Albumin (ALB) and α-fetoprotein (AFP) are genes expressed almost exclusively in cells of hepatocyte lineage. The induction of albumin or α-fetoprotein expression in non-hepatocyte cells (e.g., in human fibroblasts) can thus be used as a measure to indicate that such cells are developing or being reprogrammed to an induced hepatocyte phenotype. In order to examine the effect of 6TF mRNA transfection of human fibroblasts on the induction of albumin and α-fetoprotein gene expression, the following studies were performed. BJ fibroblasts were transfected once daily with 6TF mRNA mixture as described above. After 5 days and 9 days, gene expression levels for albumin and α-fetoprotein in the cells were determined by qPCR using methods as described above.

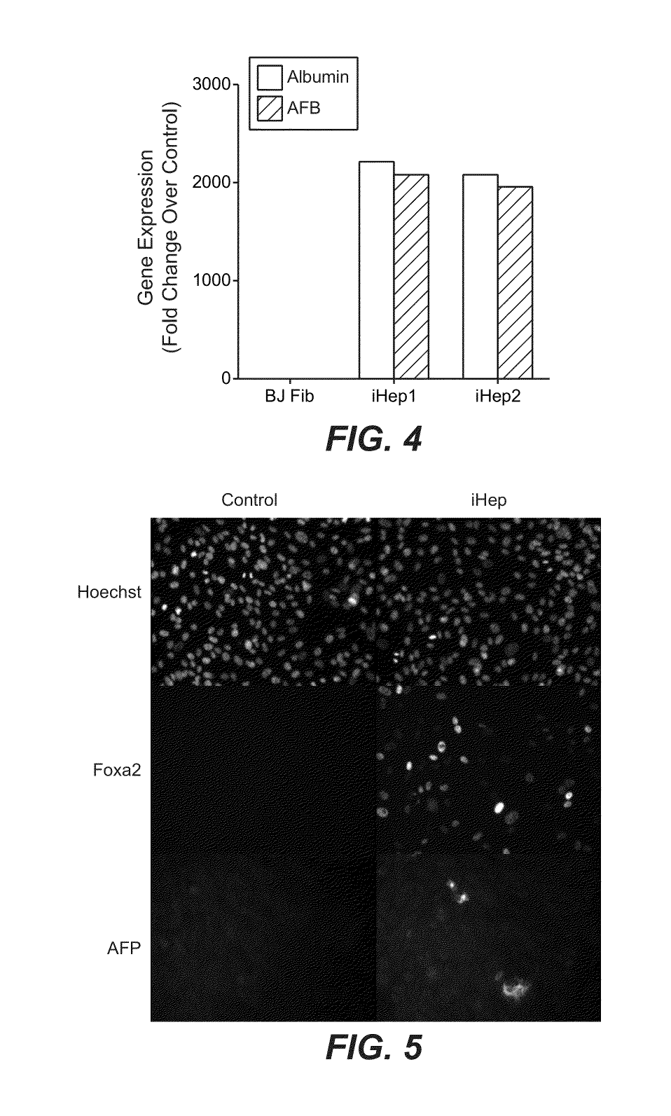

The results of a 9-day transfection experiment are shown in FIG. 4, which shows changes in albumin and α-fetoprotein gene expression in biological duplicate (from two separate tissue culture wells, labeled as iHep1 and iHep2). The results shown in FIG. 4 are presented as fold-increase in gene expression of albumin and α-fetoprotein over that observed in non-transfected control cells, where α-fetoprotein was not detected at all in control cells and an artificial cycle threshold was applied. (Control cells labeled as Fib in FIG. 4.)

As shown in FIG. 4, gene expression levels of albumin and α-fetoprotein were greatly induced in human fibroblasts transfected with 6TF mRNA mixture compared to the expression levels of these genes in non-transfected control fibroblasts. Albumin and α-fetoprotein gene expression were very low or not-detectable, respectively, in non-transfected control cells. These results showed that transfection of non-hepatocyte cells (e.g., human fibroblasts) with an mRNA mixture containing mRNAs encoding FOXA1, FOXA2, FOXA3, HNF4A, HNF1A, and GATA4 was sufficient to induce the expression of hepatocyte-specific genes (e.g., albumin and α-fetoprotein) in human fibroblasts. These results also indicated that the 6TF mRNA mixture containing mRNAs encoding FOXA1, FOXA2, FOXA3, HNF4A, HNF1A, and GATA4 contained at least one reprogramming factor or a combination of reprogramming factors capable of inducing the expression hepatocyte-specific genes in non-hepatocyte human fibroblasts and thus capable of reprogramming human fibroblasts to induced hepatocytes.

Similar results were observed in cells transfected once-daily with 6TF mRNA mixture for 5 days (data not shown).

### Example 5

### Induction of Albumin and α-Fetoprotein in Transfected Human Fibroblasts Correlates with Expression of Reprogramming Factors

To examine the correlation between expression of reprogramming factors and induction of hepatocyte-specific gene expression, the following studies were performed. BJ fibroblasts were transfected once daily with 6TF mRNA mixture as described above. After 6 days, the transfected cells were evaluated for expression of FOXA2 and α-fetoprotein using immunostaining as described above.

FIG. 5 shows immunostaining results of FOXA2 and α-fetoprotein (AFP) in human fibroblasts transfected with 6TF mRNA mixture for 6 days. As shown in FIG. 5, the majority of the transfected cells showed nuclear expression of FOXA2 protein. Within the FOXA2-positive population of cells, a population of cells existed which showed cytoplasmic expression of α-fetoprotein. Vehicle transfected fibroblasts showed no FOXA2 or α-fetoprotein expression. These results indicated that induction of α-fetoprotein following 6TF mRNA transfection is associated with a distinct population of transfected cells and correlated with the expression of FOXA2, suggesting that the induction of α-fetoprotein was specific to a true reprogramming event.

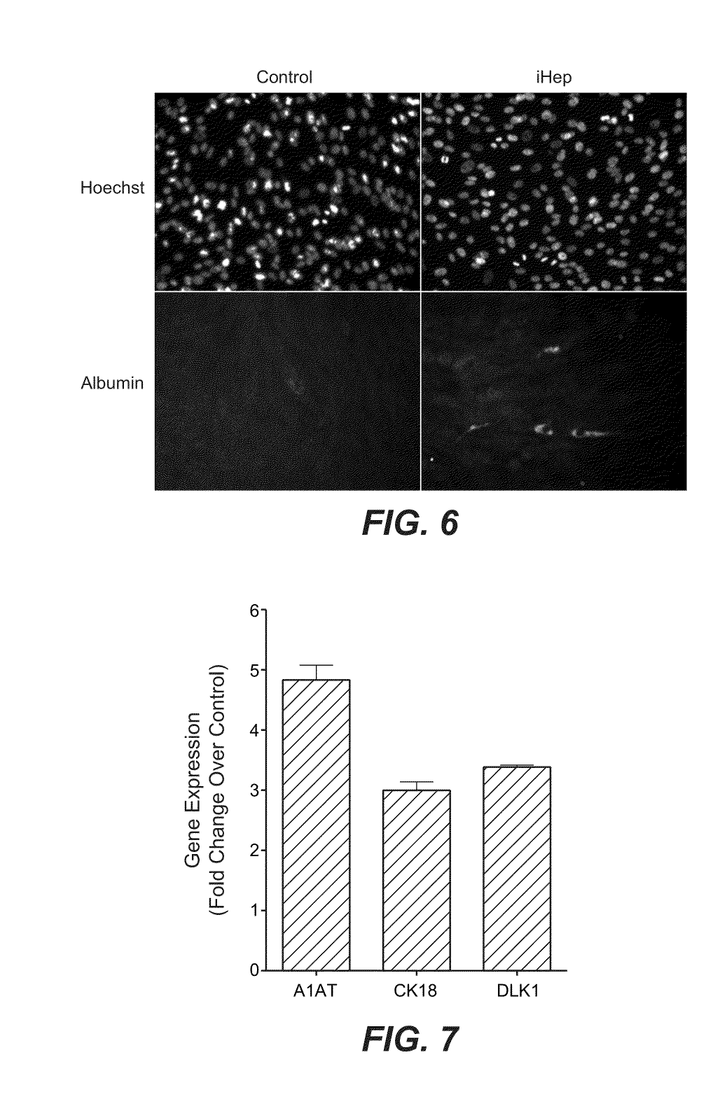

FIG. 6 shows immunostaining results of albumin in human fibroblasts transfected with 6TF mRNA mixture for 5 days. As shown in FIG. 6, a population of cells existed which showed cytoplasmic expression of albumin. Vehicle transfected fibroblasts showed no albumin expression Induced albumin expression in human fibroblasts after once-daily transfection of 6TF mRNA mixture for 5 days is indicative of a rapid reprogramming of the human fibroblasts to induced hepatocytes.

Taken together, these results suggested that induction of α-fetoprotein and albumin expression observed was not due to an overall basal up-regulation, but rather due to a distinct population of induced hepatocytes. In order for α-fetoprotein and albumin protein expression to occur at such levels in distinct cells, a discreet population of cells must have activated endogenous hepatocyte-specific transcriptional circuits, indicating that true and stable reprogramming events occurred in the epigenome of the fibroblasts, leading to the activation of hepatocyte-specific promoters and transcriptional control elements, such as those associated with expression of α-fetoprotein and albumin.

### Example 6

### Induction of α1-Anti-Trypsin, Cytokeratin 18, Delta-Like 1 Gene Expression in Human Fibroblasts Transfected with Six Transcription Factor mRNA Mixture

Alpha1-anti-trypsin (A1AT) is a protease inhibitor expressed nearly exclusively in hepatocytes. A1AT has been used as a marker for mature, functional hepatocytes in embryonic stem cell (ESC) hepatocyte differentiation. Cytokeratin 18 (CK18) is a cytoskeletal structural protein highly expressed in liver compared to other tissues. Delta-like 1 (DLK1) is a cell surface protein whose expression is highly specific to fetal liver and has been shown to be a marker for hepatic stem cells. Additionally, DLK1 and CK18 may be useful as surface markers for enrichment of induced hepatocytes. The induction of A1AT, CK18, and DLK1 expression in non-hepatocyte cells can thus be used as a measure to indicate that non-hepatocyte cells are developing or being reprogrammed into an induced hepatocyte phenotype.

In order to examine the effect of 6TF mRNA transfection of human fibroblasts on the induction or increase in gene expression of A1AT, CK18, and DLK1, the following studies were performed. BJ fibroblasts were transfected once daily with 6TF mRNA mixture as described above. After 9 days, gene expression levels for A1AT, CK18, and DLK1 in the cells were determined using qPCR as described above. As shown in FIG. 7, gene expression levels of A1AT, CK18, and DLK1 were increased in human fibroblasts transfected with 6TF mRNA mixture compared to that observed in non-hepatocyte control cells. Specifically, A1AT gene expression levels increased approximately 5-fold, while CK18 and DLK1 gene expression levels increased approximately 3-fold, over their respective gene expression levels in non-transfected control fibroblasts. These results indicated that transfection of human fibroblasts with a mixture of six transcription factor mRNAs (which includes mRNAs encoding FOXA1, FOXA2, FOXA3, HNF4A, HNF1A, and GATA4) resulted in a phenotypic change in the fibroblasts, including the induction of key hepatocyte functional (A1 AT), structural (CK18), and surface marker (DLK1) genes.

These results also indicated that the 6TF mRNA mixture containing mRNAs encoding FOXA1, FOXA2, FOXA3, HNF4A, HNF1A, and GATA4 contained at least one reprogramming factor or a combination of reprogramming factors capable of inducing the expression of hepatocyte-specific genes in non-hepatocyte human fibroblasts and thus capable of reprogramming human fibroblasts to induced hepatocytes.

### Example 7

### Induction of Hepatocyte Morphology in Human Fibroblasts Transfected with Six Transcription Factor mRNA Mixture

Multi-nucleation is observed in only a few mammalian cell types, including hepatocytes, megakaryocytes, and muscle cells. While both megakaryocytes and muscle cells often have more than 2 nuclei present in a single cell, bi-nucleation (i.e., 2 nuclei present in a single cell) is a distinctive morphological feature of hepatocytes. In contrast, fibroblasts are not bi-nucleated, unless under very specific conditions (e.g., if cytoplasmic division is blocked with cytochalasin B).

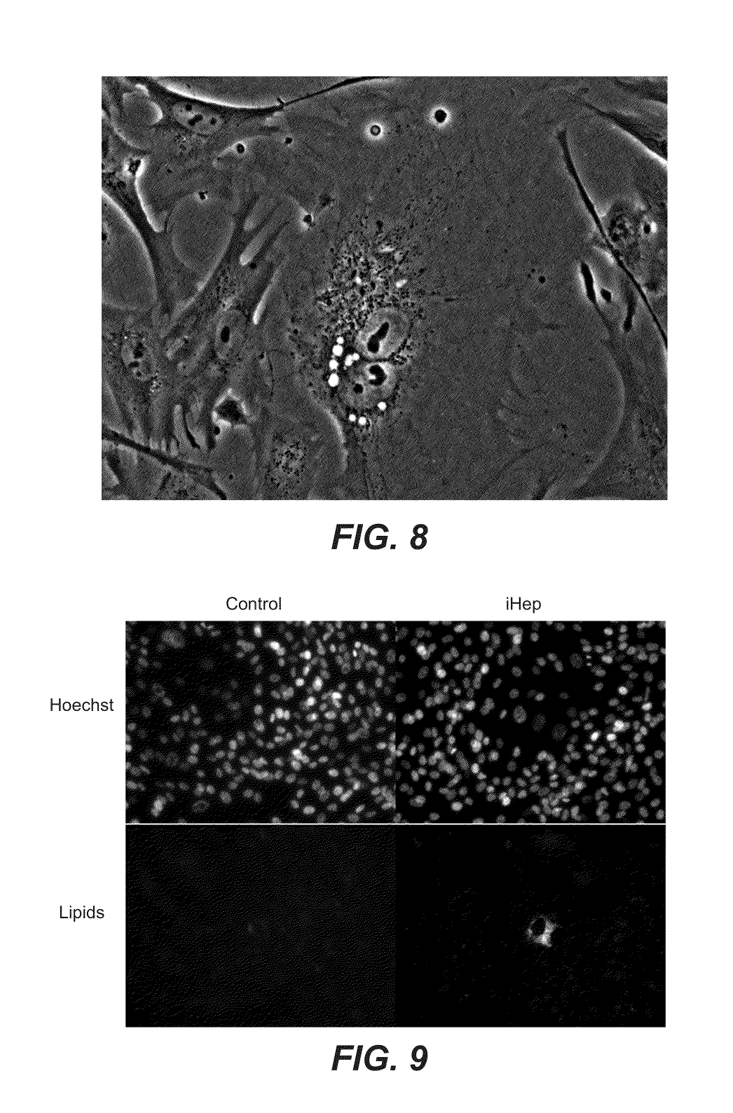

To examine morphological changes and development of multi-nucleation in human fibroblasts transfected with 6TF mRNA mixture, the following experiments were performed. BJ fibroblasts were transfected once daily with 6TF mRNA mixture as described above. After 9 days, the transfected cells were examined for changes in cell morphology. As shown in FIG. 8, BJ fibroblasts displayed a hepatocyte-like morphology following their transfection with 6TF mRNA mixture. Specifically, the transfected fibroblasts displayed bi-nucleation (i.e., the cells became bi-nucleated), a distinctive morphological feature of hepatocytes and not of fibroblasts.

Additionally, human fibroblasts transfected with 6TF mRNA mixture showed extensive vacuoles, a morphological feature common for hepatocytes but not for fibroblasts. These morphological changes suggested that the transfected human fibroblasts were reprogrammed to an induced hepatocyte phenotype. Taken together, these results showed that transfection of human fibroblasts with a mixture of six transcription factor mRNAs (encoding FOXA1, FOXA2, FOXA3, HNF4A, HNF1A, and GATA4) resulted in the fibroblasts undergoing morphological and phenotypic change in the fibroblasts, specifically bi-nucleation and extensive vacuole formation.

These results also indicated that the 6TF mRNA mixture containing mRNAs encoding FOXA1, FOXA2, FOXA3, HNF4A, HNF1A, and GATA4 at least one reprogramming factor or a combination of reprogramming factors capable of inducing hepatocyte-specific morphological and phenotypic changes in non-hepatocyte human fibroblasts and thus capable of direct reprogramming human fibroblasts to induced hepatocytes.

### Example 8

### Formation of Lipid Droplets in Human Fibroblasts Transfected with Six Transcription Factor mRNA Mixture

Lipid droplets are cellular organelles for storage of neutral lipids. Storage of neutral lipids in lipid droplets is a functional feature of mature hepatocytes and not a functional feature of fibroblasts. To investigate the appearance of lipid droplets in human fibroblasts transfected with 6TF mRNA mixture, the following experiments were performed. BJ fibroblasts were transfected once daily with 6TF mRNA mixture as described above. After 6 days, the transfected cells were stained for the presence of lipid droplets.

FIG. 9 shows distinct cells having high neutral lipid content appear as lipid droplets in human fibroblasts transfected with 6TF mRNA mixture. Lipid droplets were not observed in non-transfected human fibroblasts (vehicle control). These results indicated transfection of human fibroblasts with 6TF mRNA mixture resulted in reprogramming of the fibroblasts to an induced hepatocyte functional phenotype. These results also indicated that the 6TF mRNA mixture containing mRNAs encoding FOXA1, FOXA2, FOXA3, HNF4A, HNF1A, and GATA4 contained at least one reprogramming factor or a combination of reprogramming factors capable of inducing a hepatocyte functional phenotype in non-hepatocyte human fibroblasts and thus capable of direct reprogramming somatic cells to induced hepatocytes.

### Example 9

### Induction of Albumin and α-Fetoprotein in Human Fibroblasts Transfected with Six Transcription Factor mRNA Mixture

In order to examine the effect of 6TF mRNA transfection of human fetal lung fibroblasts on the induction of albumin and AFP gene expression, the following studies were performed. MRC-5 fibroblasts were transfected once daily for 7 days with 6TF mRNA mixture. Table 5 below provides the ng amounts of each mRNA contained in the 6TF mRNA mixture. After 7 days, gene expression levels for albumin and AFP in the cells were determined by qPCR using methods as described above.

TABLE 5

| Transcription Factor | ng mRNA |
|---|---|
| FOXA1 | 83 |
| FOXA2 | 82 |
| FOXA3 | 65 |
| HNF4A | 84 |
| HNF1A | 107 |
| GATA4 | 79 |
| Total (ng) transfected mRNA/well | 500 |

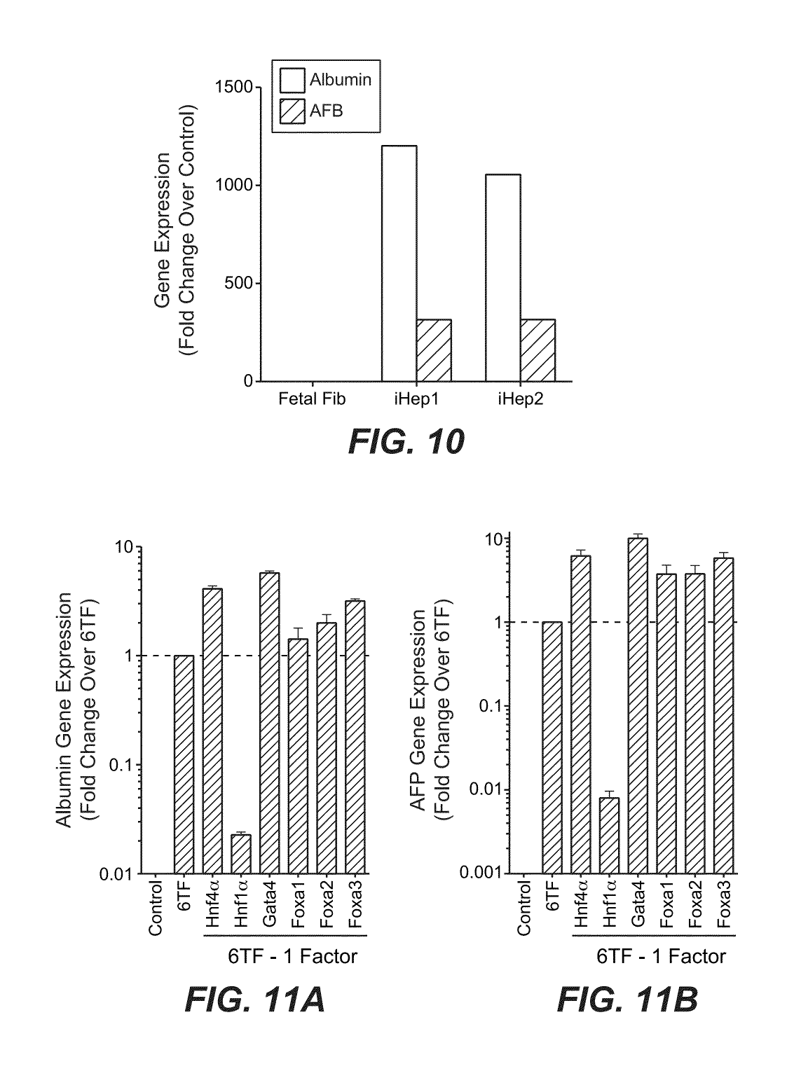

The results of these experiments are shown in FIG. 10, which shows changes in albumin and AFP gene expression. The results in FIG. 10 are presented as fold-increase in gene expression of albumin and AFP over that observed in non-transfected control cells. (Control cells labeled as Fetal Fib in FIG. 10.)

As shown in FIG. 10, gene expression levels of albumin and AFP were induced in human fetal lung fibroblasts transfected with 6TF mRNA mixture compared to the expression levels of these genes in non-transfected control fibroblasts. As observed in BJ fibroblasts (see Example 4 above), albumin and AFP gene expression were very low or not-detectable in non-transfected control MRC-5 fibroblasts. Taken together, these results showed that transfection of non-hepatocyte cells (e.g., human fetal lung fibroblasts and human foreskin fibroblasts) with an mRNA mixture containing mRNAs encoding FOXA1, FOXA2, FOXA3, HNF4A, HNF1A, and GATA4 was sufficient to induce the expression of hepatocyte-specific genes (e.g., albumin and AFP) in various human fibroblasts. These results also indicated that the 6TF mRNA mixture containing mRNAs encoding FOXA1, FOXA2, FOXA3, HNF4A, HNF1A, and GATA4 contained at least one reprogramming factor or a combination of reprogramming factors capable of inducing the expression hepatocyte-specific genes in non-hepatocyte human fibroblasts and thus capable of reprogramming human fibroblasts to induced hepatocytes.

### Example 10

### HNF1A is a Necessary Factor for Reprogramming Human Fibroblasts to Induced Hepatocytes

In order to further explore transcription factors necessary for or associated with reprogramming of human fibroblasts to induced hepatocytes, the following set of reductive experiments was performed. Various transcription factor mRNA mixtures were prepared in which one transcription factor of the 6TF mRNA mixture (6TF mRNA mixture contains mRNA encoding FOXA1, FOXA2, FOXA3, HNF4A, HNF1A, and GATA4) was not included in each of the final five transcription factor (5TF) mRNA mixtures. The 5TF mRNA mixtures used in this series of experiments contained the 6TF mixture minus FOXA1 (6TF-FOXA1), minus FOXA2 (6TF-FOXA2), minus FOXA3 (6TF-FOXA3), minus HNF4A (6TF-HNF4A), minus HNF1A (6TF-HNF1A), or minus GATA4 (6TF-GATA4). See Table 6 below for a listing of each 5TF mRNA mixture and the ng amounts of mRNA contained within each mRNA mixture.

TABLE 6
Table 6: Composition of pools and RNA transfection amounts for this series of
reductive experiments. All numbers represent ng amounts of each mRNA for each
daily transfection dose in one 6-well plate. Cells were transfected for 5 consecutive days.

| | | 6TF − | 6TF − | 6TF − | 6TF − | 6TF − | 6TF − |
|---|---|---|---|---|---|---|---|
| Transcription Factor | 6TF | FOXA1 | FOXA2 | FOXA3 | HNF4A | HNF1A | GATA4 |
| FOXA1 | 85 | 0 | 85 | 85 | 85 | 85 | 85 |
| FOXA2 | 85 | 85 | 0 | 85 | 85 | 85 | 85 |
| FOXA3 | 70 | 70 | 70 | 0 | 70 | 70 | 70 |
| HNF4A | 260 | 260 | 260 | 260 | 0 | 260 | 260 |
| HNF1A | 335 | 335 | 335 | 335 | 335 | 0 | 335 |
| GATA4 | 165 | 165 | 165 | 165 | 165 | 165 | 0 |
| Total transfected | 1,000 | 915 | 915 | 930 | 740 | 665 | 835 |
| mRNA/well (ng) | | | | | | | |

BJ fibroblasts were transfected once daily with 6TF mRNA mixture (See Table 6 above for 6TF mRNA cocktail composition) or each of the 5TF mRNA mixtures (described above and shown in Table 6) for 5 consecutive days, as described above. After 5 days, gene expression levels for albumin and AFP in the cells were determined by qPCR using methods as described above. Data is presented as fold-change in albumin or AFP gene expression levels measured in cells transfected with each mRNA mixture (see Table 6, in which mRNA encoding one transcription factor is not included in each mRNA mixture) compared to that measured in cells transfected with the complete 6TF mRNA mixture.

As shown in FIGS. 11A and 11B, human fibroblasts transfected with mRNA mixtures 6TF-FOXA1, 6TF-FOXA2, 6TF-FOXA3, 6TF-HNF4α, or 6TF-GATA4 showed albumin and AFP gene expression levels comparable or higher than that observed in cells transfected with complete 6TF mRNA mixture.

FIGS. 11A and 11B also show that human fibroblasts transfected with 6TF-HNF1A mRNA mixture showed lower gene expression levels for albumin and AFP compared to the expression levels of these genes in cells transfected with the complete 6TF mRNA mixture. This data suggested that HNF1A is a necessary transcription factor for reprogramming human fibroblasts to induced hepatocytes.

These results differ from those previously reported for reprogramming mouse fibroblasts to induced hepatocytes, indicating that the mechanisms associated with and the factors necessary and sufficient for reprogramming human fibroblasts to induced hepatocytes are different from that associated with reprogramming mouse fibroblasts to induced hepatocytes. (See Huang et al., (2011) Nature 475:386-389; and Sekiya and Suzuki (2011) Nature 475:390-393) Huang identified the combination of Gata4, Hnf1α, and Foxa3 (along with inactivation of p19^{Arf}) as being sufficient to induce murine hepatic conversion; Sekiya and Suzuki identified three specific combinations of two transcription factors, comprising Hnf4α plus Foxa1, Foxa2, or Foxa3 that induced murine hepatic conversion.

### Example 11

### Albumin and AFP Gene Expression in Human Fibroblasts Transfected with Various Transcription Factors mRNA Mixtures

In order to further explore transcription factors necessary for or associated with reprogramming of human fibroblasts to induced hepatocytes, additional reductive experiments were performed as follows. Various transcription factor mRNA mixtures were prepared in which one transcription factor of the 6TF-GATA4 mRNA mixture (a 5TF mRNA mixture containing mRNA encoding FOXA1, FOXA2, FOXA3, HNF4A, and HNF1A) was not included in each of the final four transcription factor (4TF) mRNA mixtures. The 4TF mRNA mixtures used in this series of experiments contained the 5TF mRNA mixture minus FOXA1 (5TF-FOXA1), minus FOXA2 (5TF-FOXA2), minus FOXA3 (5TF-FOXA3), minus HNF4α (5TF-HNF4α), or minus HNF1α (5TF-HNF1α). Table 7 below provides a listing of the constituents of each 4TF mRNA mixture and the ng amounts of mRNA contained within each mRNA mixture.

TABLE 7
Table 7: Composition of pools and RNA transfection amounts for this series of
reductive experiments. 5TF mRNA mixture is 6TF without Gata4. All numbers
represent ng amounts of each mRNA for each daily transfection dose in
one well (12-well plate). Cells were transfected for 5 consecutive days.

| | | 6TF − | 5TF − | 5TF − | 5TF − | 5TF − | 5TF − |
|---|---|---|---|---|---|---|---|
| Transcription Factor | 6TF | GATA4 | HNF1A | HNF4A | FOXA1 | FOXA2 | FOXA3 |
| FOXA1 | 83 | 99 | 133 | 123 | 0 | 123 | 117 |
| FOXA2 | 82 | 97 | 131 | 121 | 121 | 0 | 115 |
| FOXA3 | 65 | 77 | 104 | 97 | 96 | 96 | 0 |
| HNF4A | 84 | 99 | 133 | 0 | 124 | 123 | 117 |
| HNF1A | 107 | 127 | 0 | 159 | 158 | 158 | 150 |
| GATA4 | 79 | 0 | 0 | 0 | 0 | 0 | 0 |
| Total mRNA | 500 | 500 | 500 | 500 | 500 | 500 | 500 |
| transfected/well (ng) | | | | | | | |

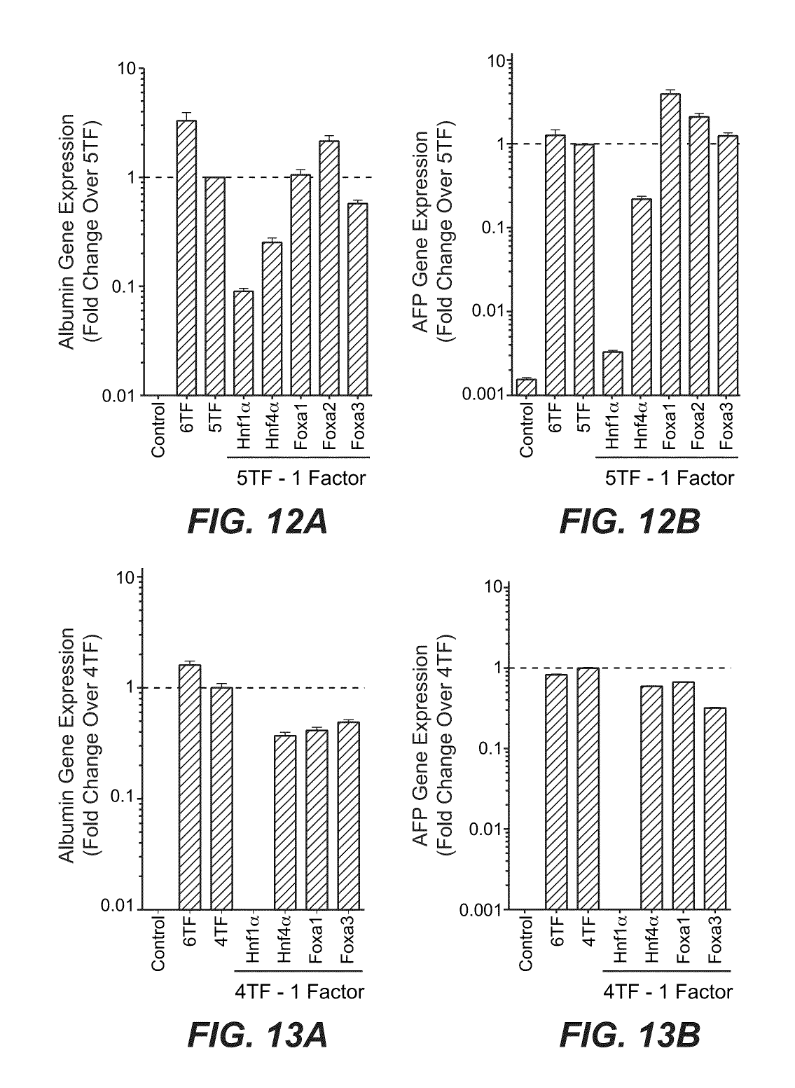

In these experiments, BJ fibroblasts cultured in 12-well tissue culture plates were used for the transfections, and the total amount of mRNA transfected per 12-well tissue culture plate was approximately 500 ng. BJ fibroblasts were transfected once daily with 6TF mRNA mixture, 6TF-GATA4 mRNA mixture, or each of the 4TF mRNA mixture shown in Table 7 for 5 consecutive days, as described above. After 5 days, gene expression levels for albumin and AFP in the transfected and control cells were determined by qPCR using methods as described above. Data is presented as fold-change in albumin or AFP gene expression levels measured in cells transfected with each mRNA mixture compared to that measured in cells transfected with 6TF-GATA4 mRNA mixture (labeled as 5TF in FIGS. 12A and 12B), which contained mRNA encoding FOXA1, FOXA2, FOXA3, HNF1A, and HNF4A, but did not contain mRNA encoding GATA4.

As shown in FIGS. 12A and 12B, the degree of induction of albumin or AFP gene expression was similar in human fibroblasts transfected with 6TF mRNA mixture compared to that observed in human fibroblasts transfected with 6TF-GATA4 mRNA mixture. These results suggested that Gata4 may not be a critical reprogramming factor associated with reprogramming human fibroblasts to induced hepatocytes.

Consistent with the experimental data described in Example 10 above, the results presented in FIGS. 12A and 12B show that HNF1A is a necessary factor for reprogramming human fibroblasts to induced hepatocytes. Specifically, human fibroblasts transfected with 5TF-HNF1A showed lower gene expression levels for both albumin and AFP compared to that observed in human fibroblasts transfected with any of the other 4TF mRNA mixtures (i.e., 5TF-FOXA1, 5TF-FOXA2, 5TF-FOXA3, 5TF-HNF4A, or 5TF-HNF1A). Additionally, albumin and AFP gene expression levels were also lower in human fibroblasts transfected with 5TF-HNF4A compared to cells transfected with the other mRNA mixtures. This data suggested that, at least in the absence of GATA4, HNF4A is a necessary factor for reprogramming human fibroblasts to induced hepatocytes.

These results differ from those previously reported for reprogramming mouse fibroblasts to induced hepatocytes, indicating that the mechanisms associated with and the factors necessary and sufficient for reprogramming human fibroblasts to induced hepatocytes are different from that associated with reprogramming mouse fibroblasts to induced hepatocytes. (See Huang et al., (2011) Nature 475:386-389; and Sekiya and Suzuki (2011) Nature 475:390-393) Huang identified the combination of Gata4, Hnf1α, and Foxa3 (along with inactivation of p19^{Arf}) as being sufficient to induce murine hepatic conversion; Sekiya and Suzuki identified three specific combinations of two transcription factors, comprising Hnf4α plus Foxa1, Foxa2, or Foxa3 that induced murine hepatic conversion.

### Example 12

### Albumin and AFP Gene Expression in Human Fibroblasts Transfected with Various Transcription Factors mRNA Mixtures

The observations described above in Examples 10 and 11 were further investigated by performing additional reductive experiments as follows. Various transcription factor mRNA mixtures were prepared in which one transcription factor of the 5TF-FOXA2 mRNA mixture (a 4TF mRNA mixture containing mRNA encoding FOXA1, FOXA3, HNF4A, and HNF1A) was not included in each of the final three transcription factor (3TF) mRNA mixtures. The 3TF mRNA mixtures used in this series of experiments contained the 5TF-FOXA2 mRNA mixture minus FOXA1 (4TF-FOXA1), minus FOXA3 (4TF-FOXA3), minus HNF4A (4TF-HNF4A), or minus HNF1A (4TF-HNF1A). Table 8 below provides a listing of the constituents of the 6FT, 4TF, and each 3TF mRNA mixture and the ng amounts of mRNA contained within each mRNA mixture.

TABLE 8
Table 8: Composition of pools and RNA transfection amounts for this series of reductive
experiments. 4TF cocktail is 6TF without Gata4 and Foxa2. All numbers represent ng
amounts of each mRNA for each daily transfection dose per 12-well tissue culture plate).
Cells were transfected once daily for 5 consecutive days.

| | | | 4TF | 4TF | 5TF | 5TF |
|---|---|---|---|---|---|---|
| Transcription Factor | 6TF | 4TF | (−HNF1A) | (−HNF4A) | (−FOXA1) | (−FOXA3) |
| FOXA1 | 83 | 123 | 179 | 163 | 0 | 152 |
| FOXA2 | 82 | 0 | 0 | 0 | 0 | 0 |
| FOXA3 | 65 | 96 | 140 | 128 | 127 | 0 |
| HNF4A | 84 | 123 | 180 | 0 | 163 | 153 |
| HNF1A | 107 | 158 | 0 | 209 | 209 | 195 |
| GATA4 | 79 | 0 | 0 | 0 | 0 | 0 |
| Total mRNA | 500 | 500 | 500 | 500 | 500 | 500 |
| transfected/well (ng) | | | | | | |

In these experiments, BJ fibroblasts cultured in 12-well tissue culture plates were used for the transfections, and the total amount of mRNA transfected per 12-well tissue culture plate was 500 ng. BJ fibroblasts were transfected once daily with 6TF mRNA cocktail, 4TF mRNA mixture, or each of the 3TF mRNA cocktails shown in Table 8 for 5 consecutive days, as described above. After 5 days, gene expression levels for albumin and AFP in the transfected cells were determined by qPCR using methods as described above. Data is presented as fold-change in albumin or AFP gene expression levels measured in cells transfected with each mRNA mixture compared to that measured in cells transfected with 4TF mRNA mixture, which contained mRNA encoding FOXA1, FOXA3, HNF1A, and HNF4A, but did not contain mRNA encoding GATA4 or FOXA2.

As shown in FIGS. 13A and 13B, human fibroblasts transfected with 4TF mRNA mixture (containing mRNA encoding FOXA1, FOXA3, HNF1A, and HNF4A) exhibited gene expression levels of albumin and AFP similar to that observed in human fibroblasts transfected with 6TF mRNA mixture (containing mRNA encoding FOXA1, FOXA2, FOXA3, HNF1A, HNF4A, and GATA4). These results suggested that FOXA1, FOXA3, HNF1A, and HNF4A were sufficient for direct reprogramming of human fibroblasts to induced hepatocytes.

Consistent with the experimental data described in Examples 10 and 11 above, the results presented in FIGS. 13A and 13B show that HNF1A is a necessary factor for reprogramming human fibroblasts to induced hepatocytes. Specifically, human fibroblasts transfected with 4TF-HNF1A showed greatly reduced gene expression levels for both albumin and AFP compared to that observed in human fibroblasts transfected with any of the other mRNA mixtures. Additionally, albumin gene expression levels were also lower in human fibroblasts transfected with 4TF-HNF1A, 4TF-FOXA1, or 4TF-FOXA3 mRNA mixtures compared to cells transfected with the other mRNA mixtures. This data suggested that HNF4A, FOXA1, and FOXA3 are important factors for reprogramming human fibroblasts to induced hepatocytes.

These results differ from those previously reported for reprogramming mouse fibroblasts to induced hepatocytes, indicating that the mechanisms associated with and the factors necessary and sufficient for reprogramming human fibroblasts to induced hepatocytes are different from that associated with reprogramming mouse fibroblasts to induced hepatocytes. (See Huang et al., (2011) Nature 475:386-389; and Sekiya and Suzuki (2011) Nature 475:390-393) Huang identified the combination of Gata4, Hnf1α, and Foxa3 (along with inactivation of p19^{Arf}) as being sufficient to induce murine hepatic conversion; Sekiya and Suzuki identified three specific combinations of two transcription factors, comprising Hnf4α plus Foxa1, Foxa2, or Foxa3 that induced murine hepatic conversion.

### Example 13

### Albumin and AFP Gene Expression in Human Fibroblasts Transfected with Various Transcription Factors mRNA Mixtures

The experimental results described above indicated that HNF1A is a necessary factor for reprogramming human fibroblasts to induced hepatocytes. In order to further explore which of the remaining 6TF mRNAs encode transcription factors necessary for reprogramming of human fibroblasts to induced hepatocytes, the following set of additive experiments was performed. In these experiments, mRNA mixtures were prepared containing mRNA encoding HNF1A or containing mRNA encoding HNF1A plus one other mRNA encoding either FOXA1, FOXA2, FOXA3, HNF4A, or GATA4. Table 9 below provides a listing of the constituents of each mRNA mixture and the ng amounts of mRNA contained within each mRNA mixture used for these experiments.

TABLE 9
Table 9: Composition of pools and RNA transfection amounts for this series of additive experiments.
All numbers represent ng amounts of each mRNA for each daily transfection dose per 6-well tissue culture plate.
Cells were transfected once daily for 5 consecutive days.

| | | HNF1A | | | | | |
|---|---|---|---|---|---|---|---|
| Transcription Factor | 6TF | (H1) | H1 + FOXA1 | H1 + FOXA2 | H1 + FOXA3 | H1 + HNF4A | H1 + GATA4 |
| FOXA1 | 85 | 0 | 85 | 0 | 0 | 0 | 0 |
| FOXA2 | 85 | 0 | 0 | 85 | 0 | 0 | 0 |
| FOXA3 | 70 | 0 | 0 | 0 | 70 | 0 | 0 |
| HNF4A | 260 | 0 | 0 | 0 | 0 | 260 | 0 |
| HNF1A | 335 | 335 | 335 | 335 | 335 | 335 | 335 |
| GATA4 | 165 | 0 | 0 | 0 | 0 | 0 | 165 |
| Total mRNA | 1000 | 335 | 420 | 420 | 405 | 595 | 500 |
| transfected/well (ng) | | | | | | | |

BJ fibroblasts in 6-well tissue culture plates were transfected once daily with each of the mRNA cocktails shown in Table 9 for 5 consecutive days, as described above. After 5 days, gene expression levels for albumin and AFP in the transfected cells were determined by qPCR using methods as described above. Data is presented as fold-change in albumin or AFP gene expression levels measured in cells transfected with each mRNA mixture compared to that measured in cells transfected with HNF1A alone.

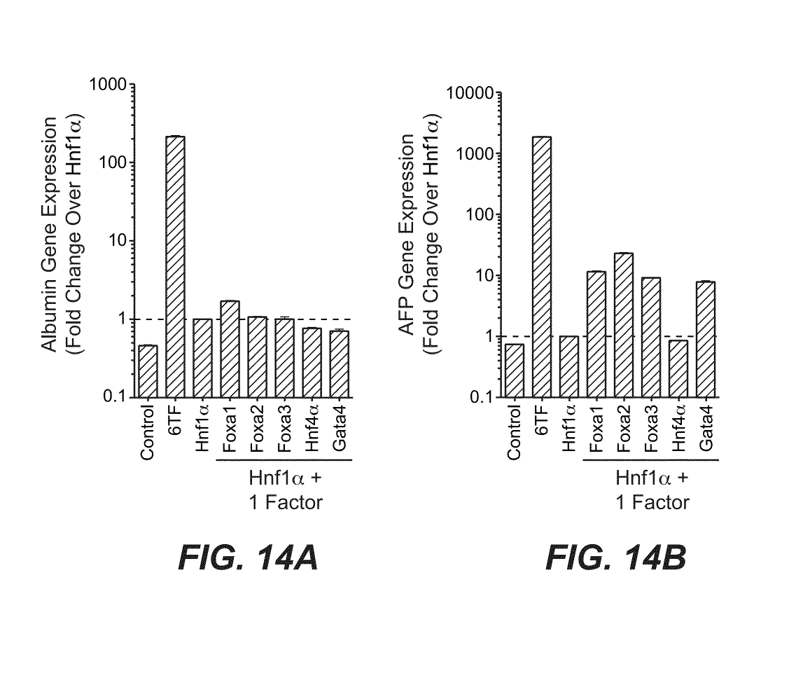

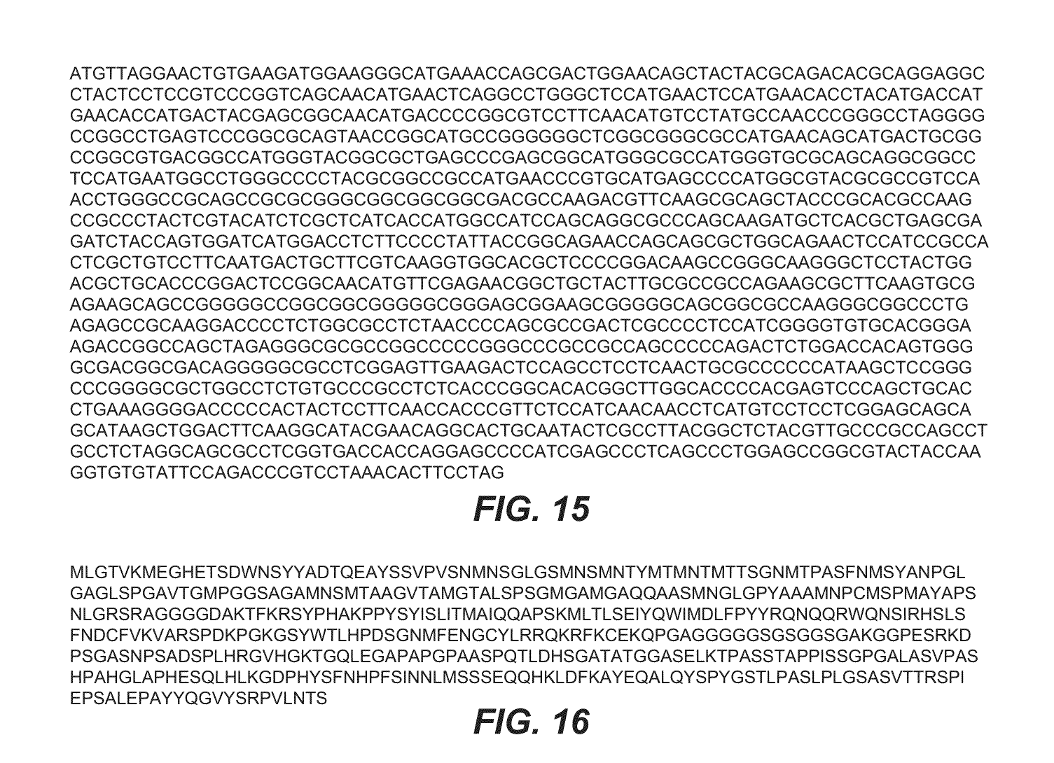

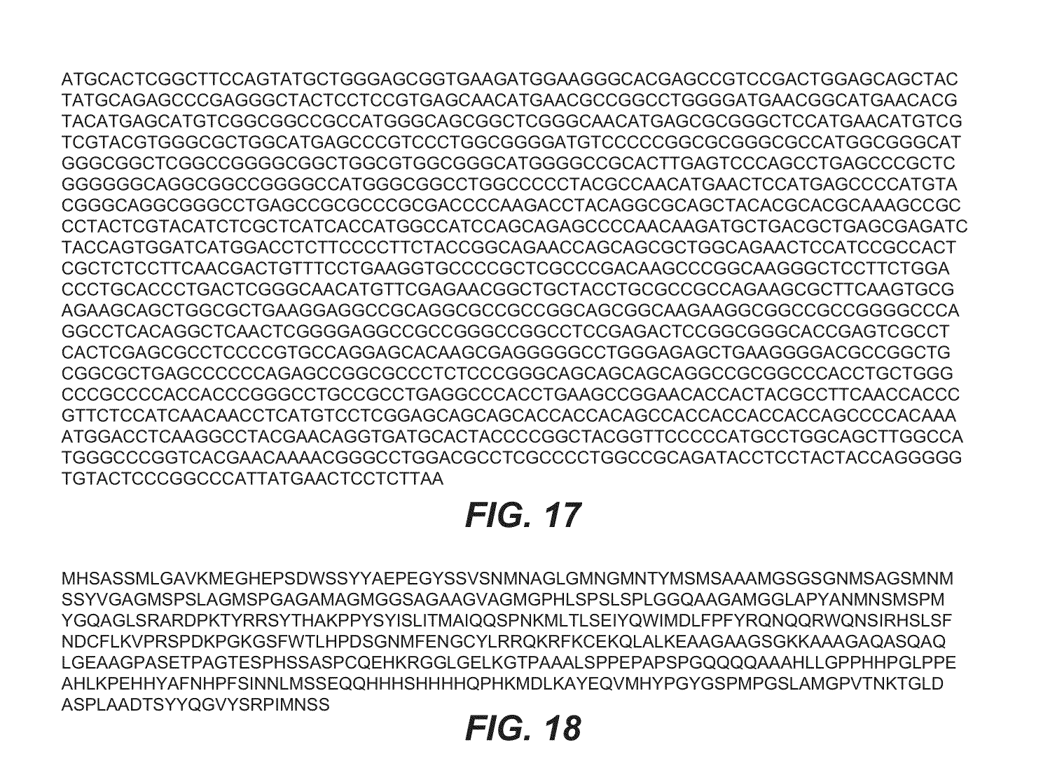

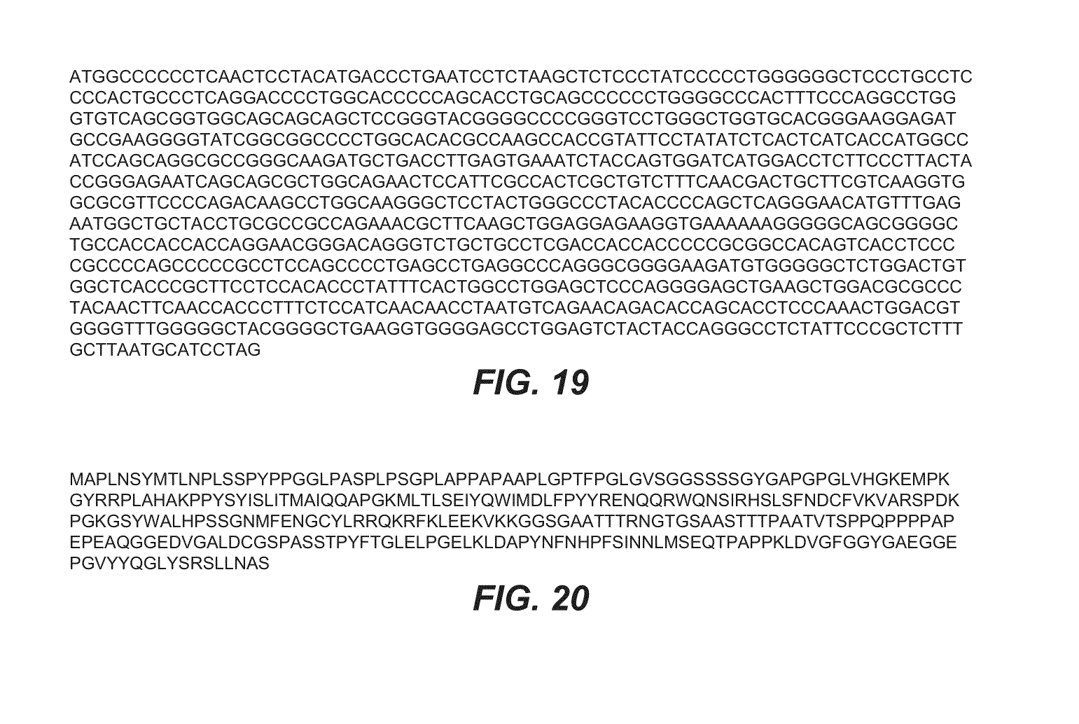

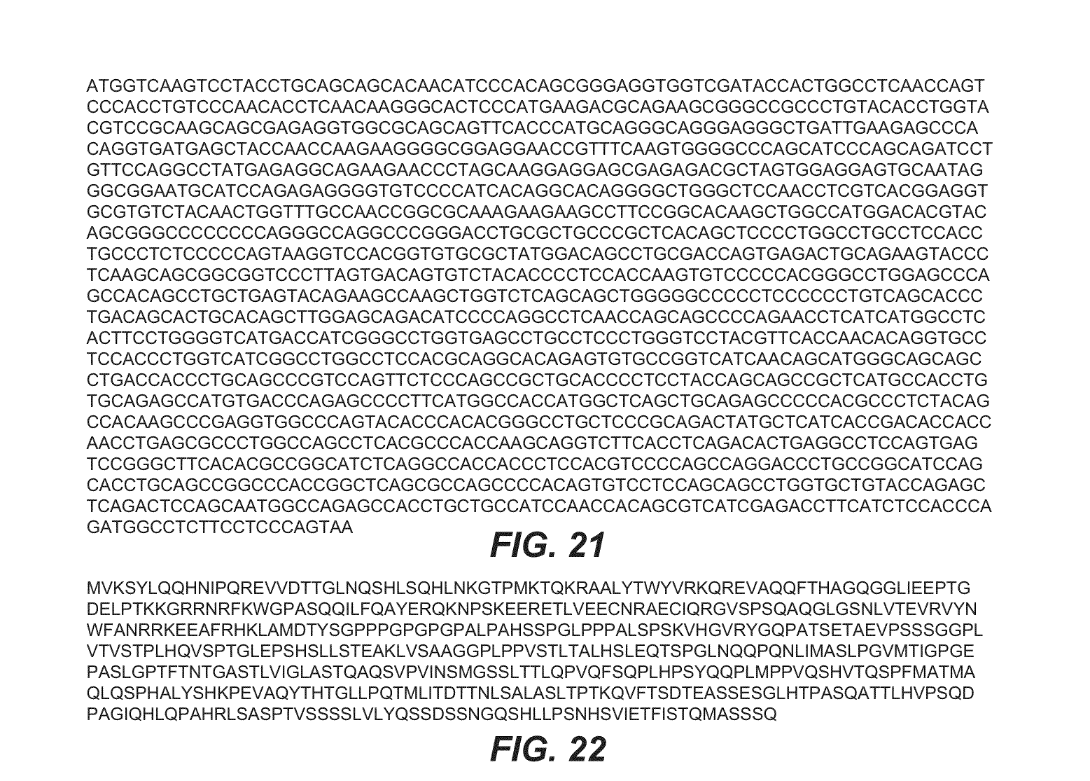

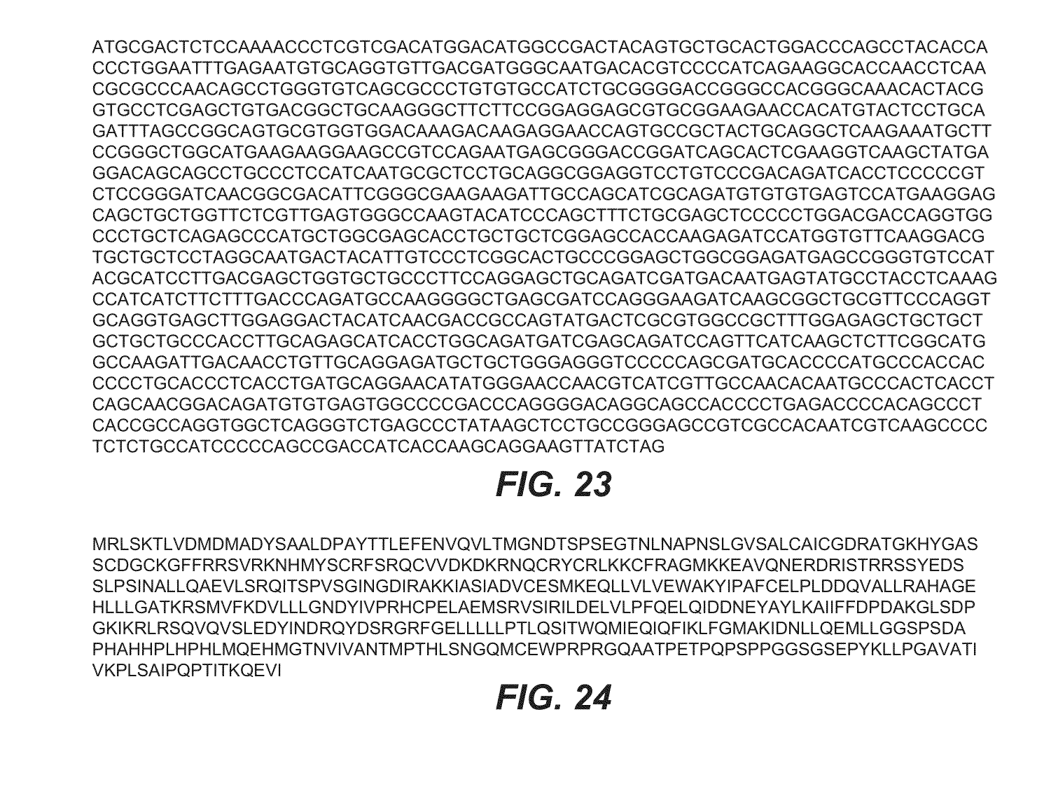

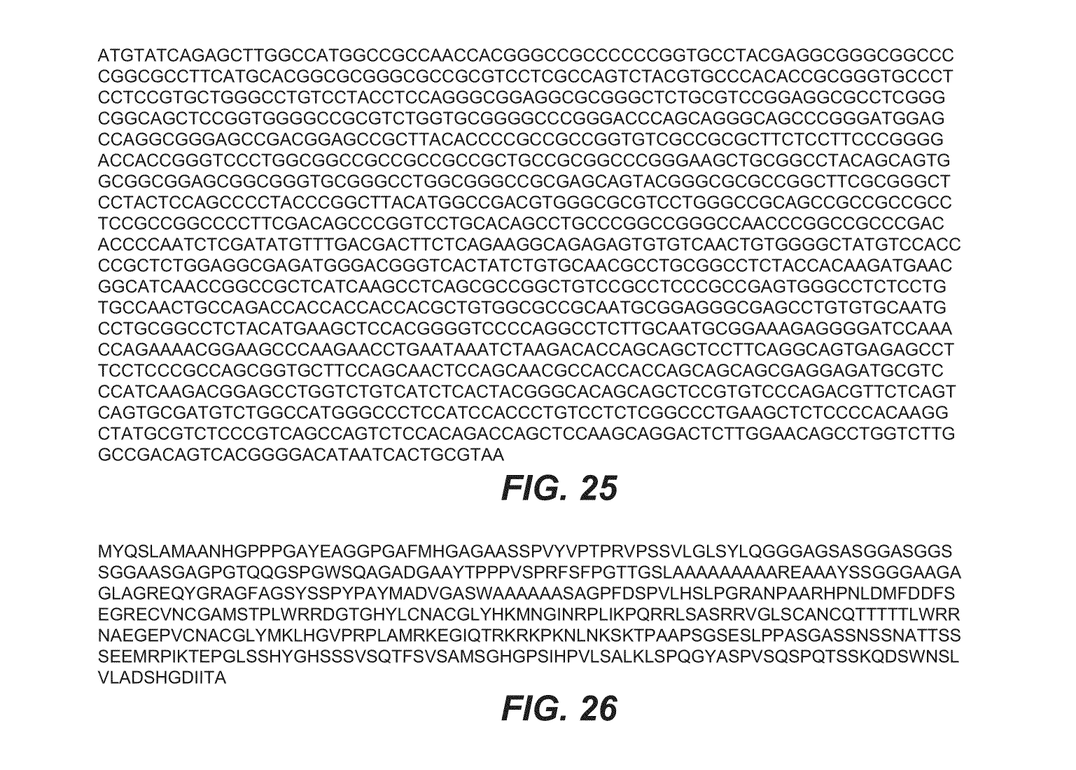

As shown in FIGS. 14A and 14B, human fibroblasts transfected with mRNA encoding only HNF1A exhibited gene expression levels of albumin and AFP slightly above that observed in non-transfected control fibroblasts. FIG. 14B also shows that human fibroblasts transfected with a mixture containing mRNA encoding HNF1A plus mRNA encoding FOXA1, FOXA2, FOXA3, or GATA4 had AFP gene expression levels greater than that observed in cells transfected with mRNA encoding HNF1A alone. Neither a single factor nor combination of two factors of the 6 transcription factors disclosed herein was sufficient to convert these human fibroblasts to induced hepatocytes to any great extent under the culture conditions used herein.

These results differ from those previously reported for reprogramming mouse fibroblasts to induced hepatocytes, indicating that the mechanisms associated with and the factors necessary and sufficient for reprogramming human fibroblasts to induced hepatocytes are different from that associated with reprogramming mouse fibroblasts to induced hepatocytes. (See Huang et al., (2011) Nature 475:386-389; and Sekiya and Suzuki (2011) Nature 475:390-393) Huang identified the combination of Gata4, Hnf1α, and Foxa3 (along with inactivation of p19^{Arf}) as being sufficient to induce murine hepatic conversion; Sekiya and Suzuki identified three specific combinations of two transcription factors, comprising Hnf4α plus Foxa1, Foxa2, or Foxa3 that induced murine hepatic conversion.

### Example 14

### Induction of Albumin and α-Fetoprotein in Human Fibroblasts Transfected with Eleven Transcription Factor mRNA Mixture

In order to examine the effect of 11TF mRNA transfection of human fibroblasts on the induction of albumin and α-fetoprotein, the following study was performed. MRC-5 fibroblasts were transfected once daily with 11TF mRNA mixture for 5 days using methods described above. After 5 days, gene expression levels for albumin and α-fetoprotein (AFP) in the cells were determined by qPCR using methods described above. 11TF mRNA mixture contained mRNA encoding C/EBPα, FOXA1, FOXA2, FOXA3, GATA4, GATA6, HHEX, HNF1α, HNF1β, HNF4α, and HNF6α.

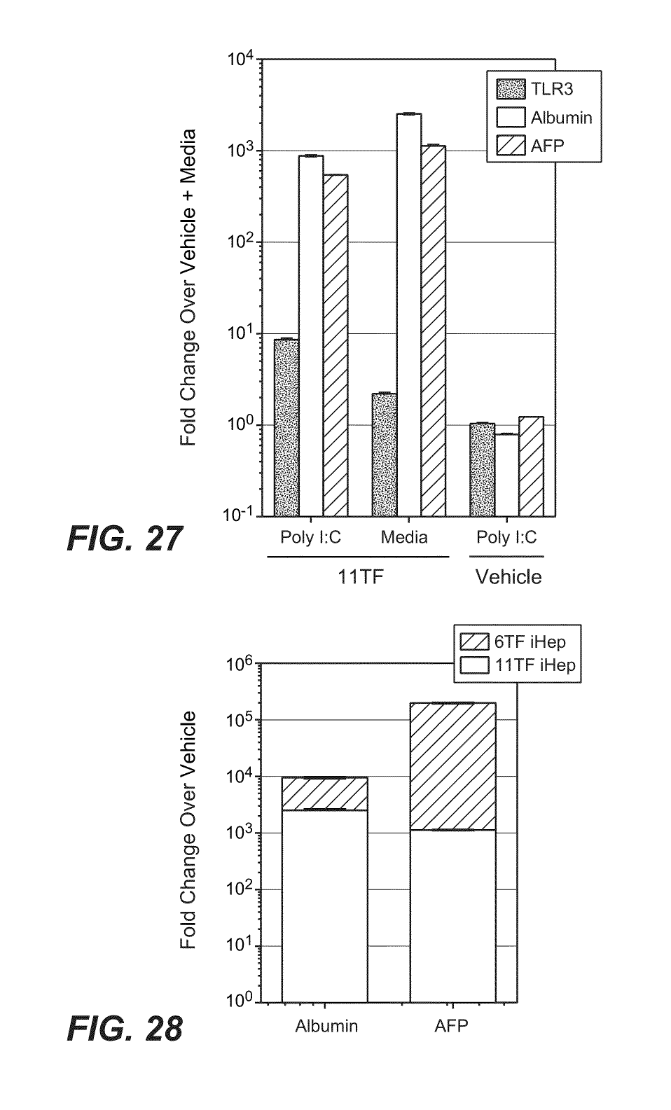

As shown in FIG. 27, gene expression and protein levels of albumin and α-fetoprotein were greatly induced in human fetal fibroblasts transfected with 11TF mRNA mixture compared to the expression levels of these genes (Poly I:C in FIG. 27) and proteins (Media in FIG. 27) in non-transfected vehicle control fibroblasts. These results showed that transfection of non-hepatocyte cells with an mRNA mixture containing mRNAs encoding C/EBPα, FOXA1, FOXA2, FOXA3, GATA4, GATA6, HHEX, HNF1α, HNF1β, HNF4α, and HNF6α was sufficient to induce the expression of hepatocyte-specific genes in human fibroblasts.

A recent report demonstrated that activation of TLR3 (toll-like receptor 3, also known as CD283) is crucial for chromatin remodeling and reprogramming. (See Lee et al., (2012) Cell 151:547-558.) Experiments were performed to examine whether 11TF mRNA mixture could activate TLR3 and to determine whether further activation would increase hepatic induction.

Experiments were performed to test whether daily supplementation with Poly I:C, a known TLR3 agonist, accompanying 11TF mRNA mixture transfection would further increase expression of albumin and AFP. Results showed that 11TF mRNA transfection alone activated TLR3 approximately 2-fold above control. (See FIG. 27.) While Poly I:C alone did not activate TLR3, Poly I:C plus 11TF mRNA transfection resulted in an 8-fold increase in expression of TLR3. This further increase in TLR3 expression, above 11TF mRNA transfection alone, was not accompanied by a corresponding increase in albumin or AFP expression, suggesting that mRNA transfection alone was sufficient to activate TLR3 for reprogramming and no further stimulation was required.

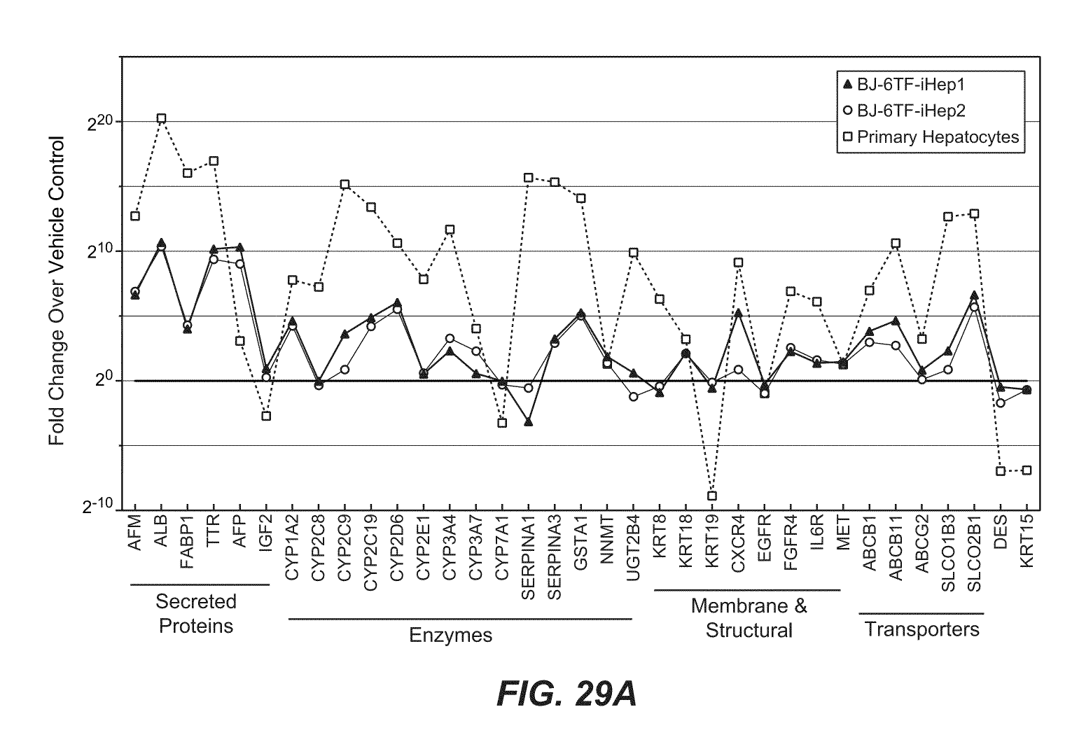

Experiments were performed to compare the reprogramming potential of 11TF mRNA mixture with 6TF mRNA mixture. Upon daily transfection of 11TF mRNA mixture and 6TF mRNA mixture for 5 days into 2 separate samples of human neonatal fibroblasts, up-regulation and activation of both albumin and AFP was observed. (See FIG. 28.) Interestingly, transfection with 6TF mRNA mixture resulted in approximately 3-fold higher albumin expression and nearly 200-fold higher AFP expression than transfection with 11TF mRNA mixture. These results indicated that 6TF mRNA mixture was more effective than 11TF mRNA mixture at activating silenced hepatocyte-specific genes and inducing direct conversion of fibroblasts to a induced hepatocytes. These results also suggested an inhibitory role for some of the 5 transcription factors that were present in 11TF mRNA mixture but not in 6TF mRNA mixture.

### Example 15

### Gene Expression Levels of Various Hepatocyte Proteins

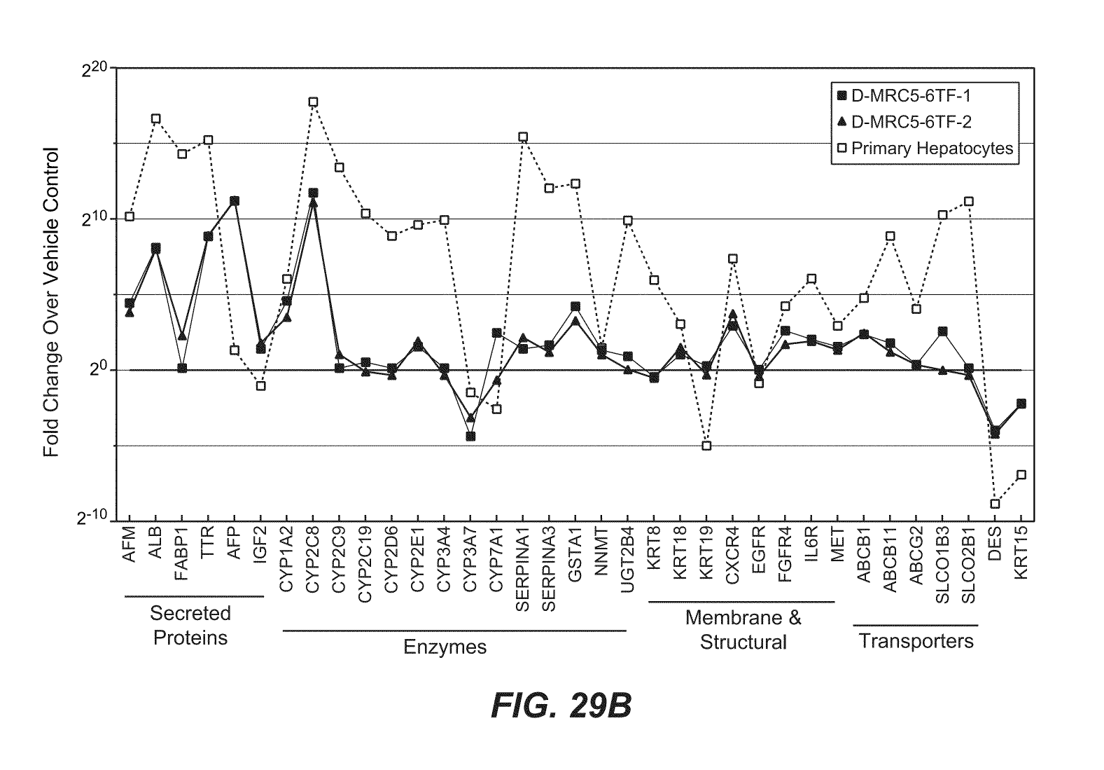

To further characterize the extent of hepatic conversion, changes in gene expression levels of genes coding for several classes of key hepatocyte proteins, including, secreted proteins, CYPs, serpins, membrane proteins, cytokeratins, and transporters were examined in human fibroblasts transfected with 6TF mRNA mixture. The results were compared to expression levels of these genes in primary hepatocytes. Duplicate experiments were performed (iHep1 and iHep 2 in FIGS. 29A and 29B). BJ fibroblasts (FIG. 29A) and MRC-5 human fibroblasts were transfected once daily with 6TF mRNA mixture for 5 days.

Generally, gene expression levels in the transfected cells tracked with that observed in primary hepatocytes. Notably, CYP expression, including CYP3A4, was observed at relatively high levels after only 5 days of transfection with 6TF. (See FIGS. 29A and 29B.) Expression of most genes measured was approximately 1,000-fold higher in pure primary hepatocytes than that observed in 6TF-iHeps, consistent with the prior observation of the frequency of albumin and AFP high-expressing cells being 1:1,000 to 1:10,000.

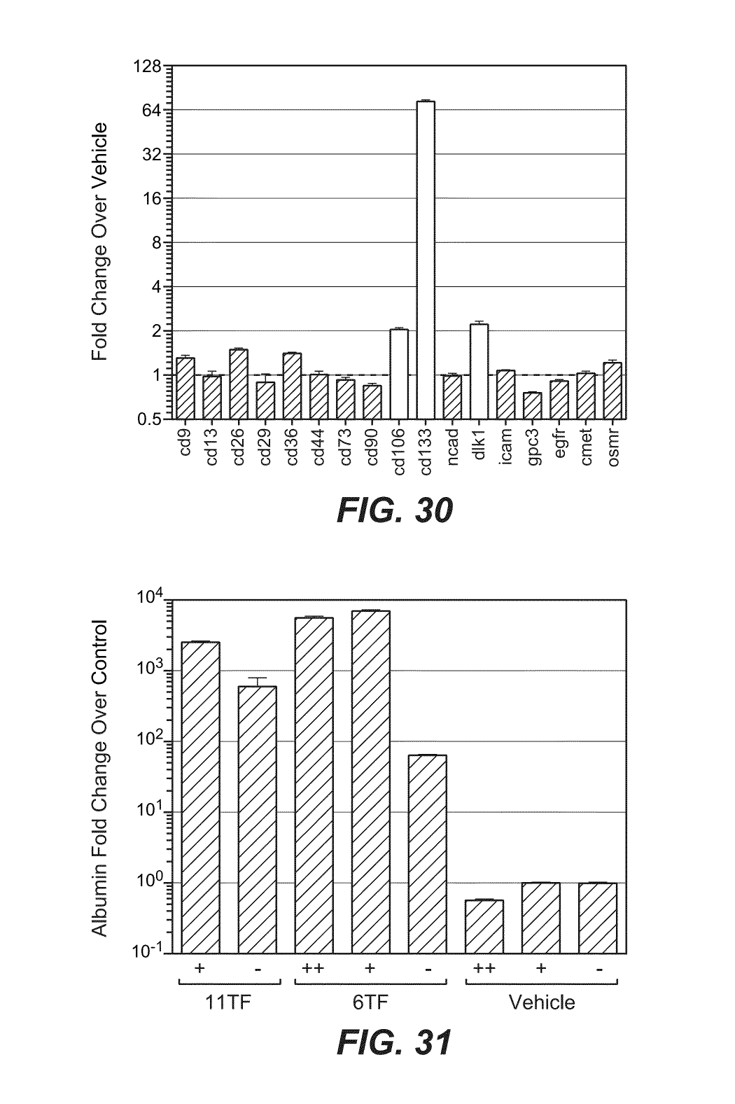

Changes in the expression of cell surface markers were also examined following transfection with 6TF mRNA mixture. As shown in FIG. 30, cell surface proteins CD106, CD133, and DLK1 showed increased gene expression levels compared to that observed in vehicle control-transfected fibroblasts.

### Example 16

### Effect of Glucocorticoid on Direct Reprogramming Human Fibroblasts to Induced Hepatocytes

Many hepatocyte genes can only be activated via simultaneous binding to glucocorticoid response elements (GREs) as well as specific transcription factor binding sites (Phuc et al., (2005) PLoS Genetics 1:e16). Studies were performed to examine whether synergistic stimulation of the glucocorticoid receptor (GR) and transfection of hepatic master transcription factors during reprogramming was crucial to the success of hepatic induction and generation of induced hepatocytes by direct reprogramming. To examine this, the effects of removal of dexamethasone, a strong GR agonist, on reprogramming was investigated.

Human fibroblasts were transfected once daily for 5 days with either 11TF mRNA mixture or 6TF mRNA mixture in the presence or absence of dexamethasone supplementation (either 0.1 μM (+ in FIG. 31), which is the standard concentration used in culture media, or 1.0 μM (++ in FIG. 31)) in the culture media. Either concentration of dexamethasone alone (without concurrent transfection with 11TF or 6TF mRNA mixtures) did not result in hepatic induction, as measured by induction of albumin gene expression. However, when dexamethasone was not included in the culture media during transfection with 6TF mRNA mixture, a reduced induced hepatocyte conversion rate of approximately 100-fold was observed. (See FIG. 31.)

These results suggested that, as expected, glucocorticoid receptor (GR) activation and binding to glucocorticoid response elements is a necessary component of the transcriptional machinery required to cause reprogramming of a non-hepatocyte to an induced hepatocyte. Interestingly, samples reprogrammed with 11TF mRNA mixture were not as dramatically affected by dexamethasone removal as observed with 6TF mRNA mixture, showing only a 5-fold decrease in albumin. These results suggested that one or more of the 5 additional transcription factors present in 11TF mRNA mixture either achieved GR activation indirectly or aided hepatic induction by a GR-independent mechanism. Of note, a 10-fold increase in dexamethasone supplementation (++ in FIG. 31) was not accompanied by a corresponding increase in albumin expression, and in fact resulted in slightly lower albumin expression, possibly due to mild toxicity at such a high concentration. This suggested that dexamethasone was saturating GR at the initial concentration of 0.1 μM and no further supplementation would be beneficial.

### Example 17

### HNF1α is a Master Regulator of Hepatocyte Reprogramming

Examples 10, 11, 12, and 13 above showed the requirement of some transcription factors over others in hepatocyte reprogramming, in particular HNF1α. In particular, we expected that the importance of HNF1a was due to a lack of compensatory activation from the other 5 transcription factors of the 6TF mRNA mixture. To test this, gene expression levels of each of the 6 transcription factors of 6TF mRNA mixture were measured when the transcription factor of interest was left out of the reprogramming cocktail and only the other 5 transcription factors were transfected for reprogramming. This would provide a direct measure of how much each ‘missing’ transcription factor was compensated via activation by the 5 remaining transcription factors.

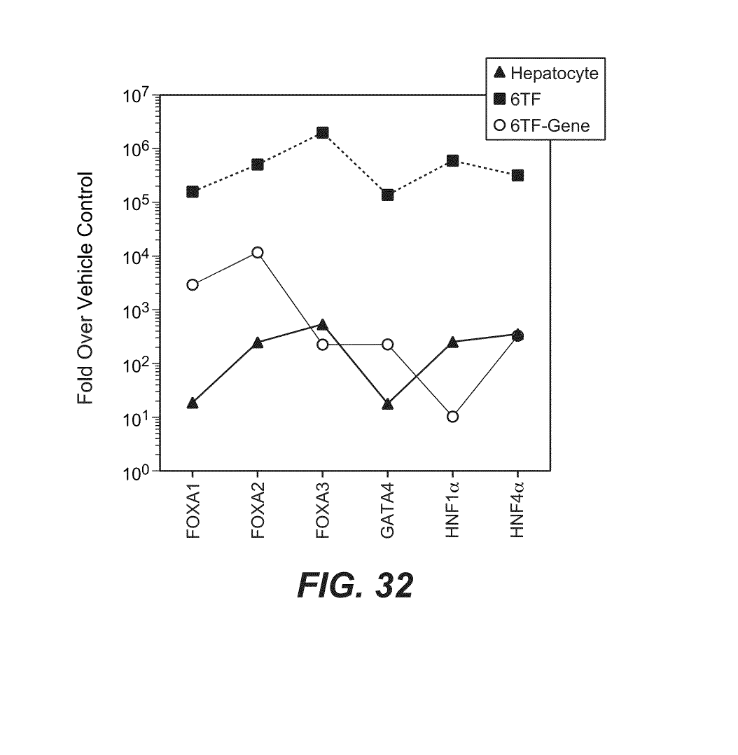

As shown in FIG. 32, HNF1a was the least compensated for among the 6TF transcription factors, the expression of which increased only 10-fold above the endogenous expression level in human fibroblasts and well below the levels observed in human primary hepatocytes. All other transcription factors were compensated by the remaining 5 transcription factors in each respective sample. In fact, all other transcription factors, except for FOXA3, were compensated to such an extent by the other transcription factors that they were elevated either to the levels observed in primary hepatocytes, as was the case for HNF4a, or to much higher levels, as was for FOXA1, FOXA2, and GATA4. (See FIG. 32.) These results indicated that HNF1α is likely a master transcription factor responsible for hepatocyte induction in human cells.

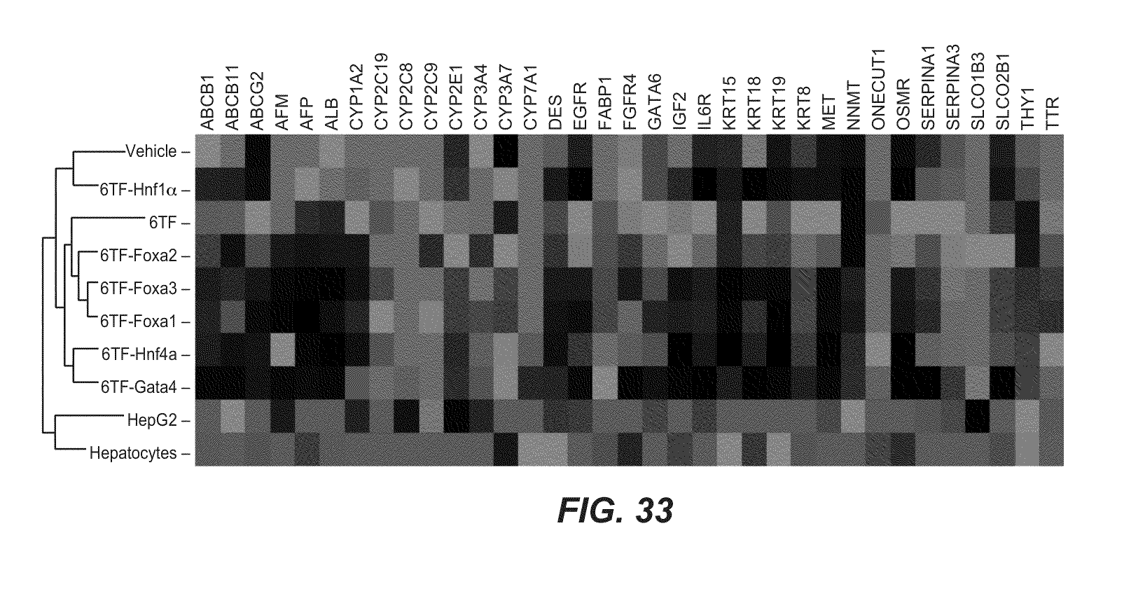

To further examine the importance of HNF1a on hepatocyte reprogramming, a cluster analysis of approximately 35 critical hepatic genes was performed. (See FIG. 33.) Cell samples for this analysis included the fibroblast transcription factor reduction samples, where 1 transcription factor was removed from the 6TF mRNA mixture, fibroblasts transfected with the 6TF mRNA mixture, a vehicle control sample, primary human hepatocytes, and HepG2 human hepatoma cell line. As expected, HepG2 cells clustered closest to the primary hepatocytes, followed by that observed in the heterogeneous 6TF sample and most of the reduction samples that were comprised of a mix of induced hepatocyte-like cells and fibroblasts. Furthest from the hepatocytes were the vehicle control sample and reduction sample missing HNF1a, both of which clustered tightly together, as they were comprised of non-induced fibroblasts. These results further demonstrated the requirement of HNF1a in the activation of this panel of critical hepatic genes, thus leading to hepatocyte induction. (See FIG. 33.)

### Example 18

### Gene Expression Levels of Various Hepatocyte Proteins

Studies were performed to examine the expression of an array of hepatocyte-specific genes as follows. In these experiments, either human fetal BJ-fibroblasts or MRC5 embryonic stem cells were transfected with 6TF mRNA mixture. Without any subsequent enrichment of transformed cells, qPCR was performed on the reprogrammed cells and on primary hepatocytes to examine and compare gene expression levels of 33 hepatocyte-specific genes.

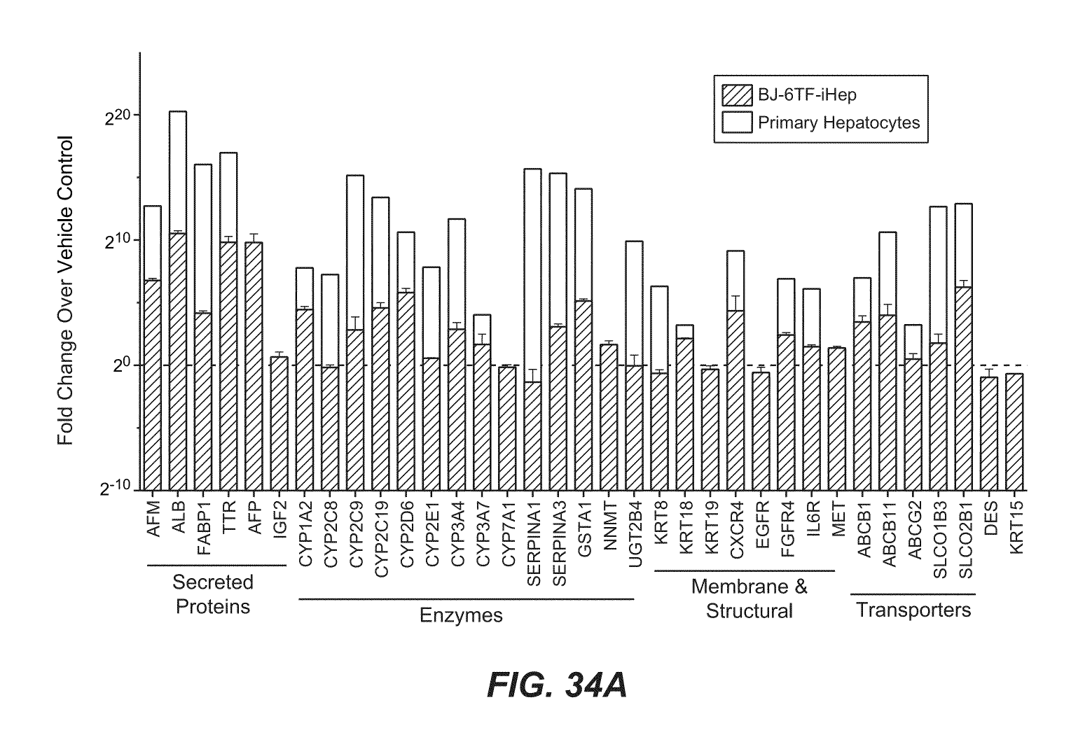

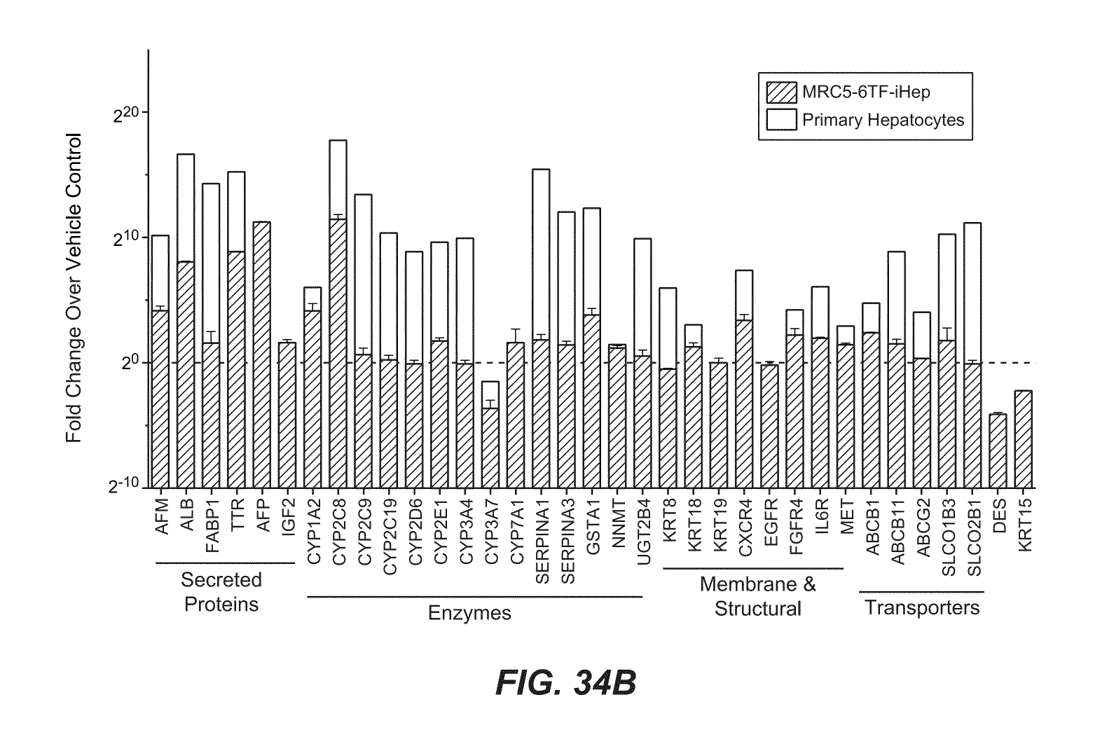

As shown in FIGS. 34A and 34B, numerous hepatocyte-specific genes were up-regulated following transfection with 6TF mRNA mixture in both human fetal fibroblasts and in human embryonic stem cells. These genes included secretory proteins (e.g., FABP1), enzymes (e.g., CYP3A4), and transporters (e.g., ABCB1). While the expression levels of these genes in reprogrammed cells was not as robust as that observed in primary hepatocytes, these results demonstrated that many hepatocyte genes were up-regulated in the reprogrammed cells compared to that observed in control human fetal fibroblasts and human embryonic stem cells. (See FIGS. 34A and 34B, which shows fold-increased in gene expression levels over that observed in vehicle-treated control human fetal fibroblasts and human embryonic stem cells, respectively.)

### Example 19

### Transcriptone and Small RNA Analysis of Reprogrammed Cells

The transcription factors included within 6TF mRNA mixture and 11TF mRNA mixture are not exclusively expressed in hepatocytes; there expression is also associated with other developmentally-related tissues, particularly endodermal tissues. (See, e.g., Stainier (2002) Genes & Development 16:893-907.) In order to examine additional changes occurring during the initial reprogramming stages using the reprogramming methods of the present invention, an analysis of the complete transcriptome and small RNA sequencing on human fetal fibroblasts reprogrammed for 5 days with 11TF mRNA mixture, 6TF mRNA mixture, or vehicle control was performed (without any subsequent enrichment of transformed cells).

Transcriptome and small RNA sequencing was performed as follows. Total RNA samples obtained from transfected cells were extracted by miRNAeasy Mini Kit (Qiagen). Transcriptome sequencing (4G clean data) and small RNA (20 mil clean reads) sequencing were performed by Bejing Genomics Institute Americas (BGI Americas, Cambridge, Mass.). RNA samples were processed using a standard BGI workflow, which included RNA quality assessment, library construction, library validation, clustering, sequencing on Illumina HiSeq™ 2000, and standard bioinformatics analysis. Significance of differentially-expressed genes was determined by BGI by calculating the false discovery rate (FDR) for each gene using Bonferroni correction of p-values. FDR values under 0.001 were deemed significant. In the figures where log 2 ratios are plotted, such values correspond to the log 2 of the appropriate RPKM values. Tissue specific genes were identified using the Tissue-specific Gene Expression and Regulation (TiGER) database.

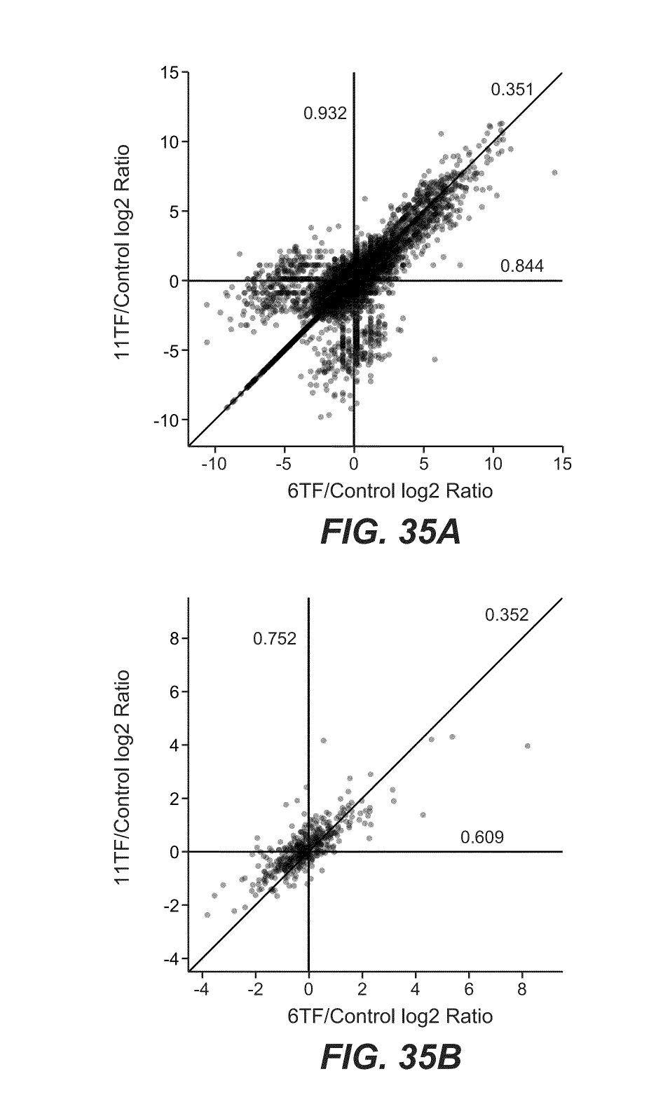

The results showed that mean dispersion (md) of expressed genes away from that of control was directionally similar in cells transfected with 6TF mRNA mixture or 11TF mRNA mixture (md=0.351), but greater in 6TF (md=0.932) than in 11TF (md=0.844) samples (FIG. 35A). The results showed that mean dispersion (md) of expressed small RNAs away from that of control was directionally similar in cells transfected with 6TF or 11TF mRNA mixtures (md=0.352), but greater in 6TF (md=0.752) than in 11TF (md=0.609) samples (FIG. 35B).

These results confirmed our findings (as described above) that 6TF mRNA mixture is more effective than 11TF mRNA mixture for reprogramming human non-hepatocyte cells to a human hepatocyte state (i.e., to an induced hepatocyte). Global gene expression analysis of human fetal fibroblasts transfected with 6TF mRNA mixture compared to that of control fibroblasts showed that hepatocyte-specific genes, such as, for example, FBB, APOA1, and SERPINA1, were dramatically up-regulated from nearly undetectable levels; fibroblast-specific genes, such as, for example, FSP1, DES, and VIM, were down-regulated; and pluripotency genes, such as, for example, OCT4 and NANOG, were unchanged (data not shown). Additionally, genes essential to liver repair during injury, including CXCL9, CXCL10, CXCL11, and ODC1 were also activated (data not shown). (See Wasmuth et al., (2009) Gastroenterology 137:309-319 and Ohtake et al., (2008) Cell Biochemistry and Function 26:259-365.)

Hepatocyte-associated miRNAs were also upregulated in human fetal hepatocytes transfected with 6TF mRNA mixture. Notably, miR-122, which accounts for over 70% of the total miRNA content of hepatocytes (Girard et al., (2008) J Hepatology 48:648-656), was among the most up-regulated miRNA observed in human fetal fibroblasts transfected with 6TF mRNA mixture (data not shown).

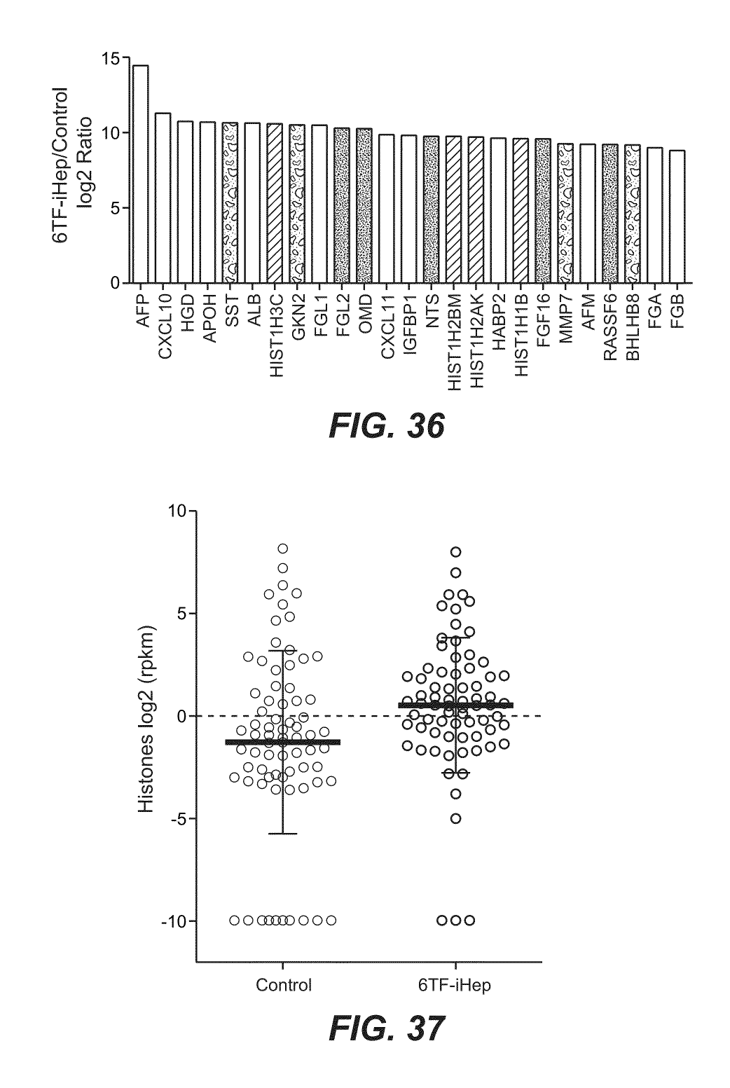

Of the top 25 most up-regulated genes in 6TF mRNA-transfected samples, 12 genes are ascribed as liver-specific or liver injury-associated, whereas only 4 genes were associated with other endodermal tissues. (See FIG. 36) Interestingly, 4 of these 25 most up-regulated genes encoded histones, a trend also observed globally, as genes encoding histones were expressed significantly higher in non-hepatocyte cells transfected with 6TF mRNA mixture than that observed in control cells. (See FIG. 37.)

### Example 20

### Endoderm Gene Expression

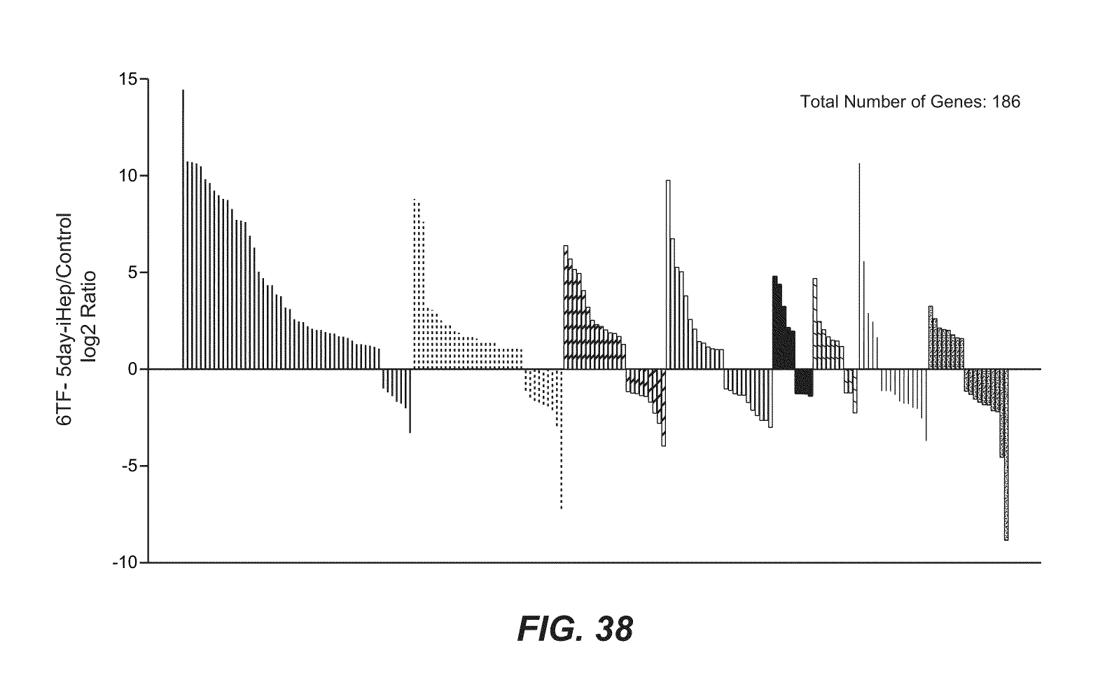

In an effort to understand global changes in tissue-specific gene expression in these early reprogramming events, the Tissue-specific Gene Expression and Regulation (TiGER) database (Liu et al., (2008) BMC Bioinformatics 9:271) was used to annotate genes as specific to a major endodermal tissue (e.g., colon, liver, lung, pancreas, small intestine, and stomach), placental tissue as proxy for cellular immaturity, and soft tissue as proxy for fibroblasts. In human non-hepatocyte cells (human fetal fibroblasts) transfected with 6TF mRNA mixture, expression of genes annotated as liver-specific (R²=0.184) were most divergent from control, expression of pancreas-specific genes a distant second (R²=0.488), and expression of soft tissue-specific genes remained nearly unchanged (R²=0.860). (Data not shown.) Examination of exclusively tissue-specific genes that were up-regulated or down-regulated more than 2-fold revealed that dispersion of liver-specific genes resulted mostly from up-regulation of gene expression, whereas dispersion of genes specific to other endodermal tissue followed no particular direction, and soft tissue genes were mostly down-regulated. (See FIG. 38; gene expression levels from tissues shown in FIG. 38 include, from left to right, liver, placenta, colon, stomach, lung, small intestine, pancreas, soft tissue.) Furthermore, many up-regulated placenta-specific genes could be associated with gene expression patterns of fetal liver. Similar results, although to a lesser extent, were observed in human fetal hepatocytes transfected with 11TF mRNA mixture (data not shown). Taken together, these results showed that transfection of human non-hepatocyte cells with 6TF mRNA mixture reprogrammed the cells preferentially toward a human hepatocyte state over other closely related cell types of endoderm origin and away from a soft tissue state.

### Example 21

### Reprogramming Media

A reprogramming media was developed which showed optimal reprogramming results; this media was used in the reprogramming experiments described herein. The reprogramming media was developed by screening an array of growth factors, small molecules, basal media, and tissue culture dish coatings for the ability to activate hepatic genes in human CD34+ bone marrow cells, which have weak hepatic transdifferentiation potential (data not shown). (See Jang et al. (2004) Nature Cell Biology 6:532-539 and Theise et al. (2000) Hepatology 32:11-16.) The reprogramming media contained DMEM/F12+Glutamax media (Invitrogen, Carlsbad, Calif.) with 10% HyClone FBS (Thermo Scientific, Waltham, Mass.), 1% Insulin-Transferrin-Selenium (Invitrogen), 1% MEM Non-Essential Amino Acids (Invitrogen), and 5 mM HEPES buffer, further supplemented with 20 ng/ml human hepatocyte growth factor (HGF), 20 ng/ml epidermal growth factor (EGF), 20 ng/ml fibroblast growth factor 2 (FGF2) (Peprotech, Rocky Hill, N.J.), 200 ng/mL B18R (eBioscience, San Diego, Calif.), and 0.1 μM dexamethasone (Sigma). All cell culture was performed in antibiotic-free culture media.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for producing an induced hepatocyte from a non-hepatocyte cell, the method comprising providing a non-hepatocyte cell, contacting the non-hepatocyte with an agent which increases or induces the expression or activity of one or more reprogramming factors, and culturing the non-hepatocyte cell under conditions suitable for reprogramming a non-hepatocyte cell to an induced hepatocyte, thereby producing an induced hepatocyte from the non-hepatocyte cell.

2. A method for producing an induced hepatocyte from a non-hepatocyte cell, the method comprising providing a non-hepatocyte cell, introducing into the non-hepatocyte cell a nucleic acid or protein preparation which encodes or provides one or more reprogramming factors, and culturing the non-hepatocyte cell under conditions suitable for reprogramming a non-hepatocyte cell to an induced hepatocyte, thereby producing an induced hepatocyte from the non-hepatocyte cell.

3. The method of claim 1 or claim 2, wherein the one or more reprogramming factors is selected from the group consisting of CEBPA, GATA6, HHEX, HNF1B, HNF6A, FOXA1, FOXA2, FOXA3, GATA4, HNF1A, and HNF4A.

4. The method of claim 1 or claim 2, wherein the one or more reprogramming factors is selected from the group consisting of FOXA1, FOXA2, FOXA3, HNF1A, HNF4A, and GATA4.

5. The method of claim 1 or claim 2, wherein the reprogramming factors comprise CEBPA, GATA6, HHEX, HNF1B, HNF6A, FOXA1, FOXA2, FOXA3, GATA4, HNF1A, and HNF4A.

6. The method of claim 1 or claim 2, wherein the reprogramming factors comprise FOXA1, FOXA2, FOXA3, HNF1A, HNF4A, and GATA4.

7. The method of claim 1 or claim 2, wherein the reprogramming factors comprise FOXA1, FOXA2, FOXA3, HNF1A, and HNF4A.

8. The method of claim 1 or claim 2, wherein the reprogramming factors comprise FOXA1, FOXA3, HNF1A, and HNF4A.

9. The method of claim 1 or claim 2, wherein the reprogramming factors comprise FOXA1, FOXA2, HNF1A, and HNF4A.

10. The method of claim 1 or claim 2, wherein the reprogramming factors comprise FOXA2, FOXA3, HNF1A, and HNF4A.

11. The method of claim 1 or claim 2, wherein the reprogramming factors comprise FOXA1, HNF1A, and HNF4A.

12. The method of claim 1 or claim 2, wherein the reprogramming factors comprise FOXA3, HNF1A, and HNF4A.

13. The method of claim 1 or claim 2, wherein the reprogramming factors comprise FOXA2, HNF1A, and HNF4A.

14. The method of claim 1 or claim 2, wherein the reprogramming factors comprise FOXA1, FOXA3, and HNF1A.

15. The method of claim 1 or claim 2, wherein the reprogramming factors comprise FOXA1, FOXA2, and HNF1A.

16. The method of claim 1 or claim 2, wherein the reprogramming factors comprise FOXA2, FOXA3, and HNF1A.

17. The method of claim 2, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, a nucleic acid sequence of SEQ ID NO:41, and a nucleic acid sequence comprising SEQ ID NO:43.

18. The method of claim 2, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, and a nucleic acid sequence of SEQ ID NO:41.

19. The method of claim 2, wherein the nucleic acid preparation comprises nucleic acid molecules comprising a nucleic acid sequence comprising SEQ ID NO:33, a nucleic acid sequence comprising SEQ ID NO:37, a nucleic acid sequence comprising SEQ ID NO:39, and a nucleic acid sequence of SEQ ID NO:41.

20. The method of claim 2, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:39, and a nucleic acid sequence of SEQ ID NO:41.

21. The method of claim 2, wherein the nucleic acid preparation comprises nucleic acid molecules comprising a nucleic acid sequence comprising SEQ ID NO:35, a nucleic acid sequence comprising SEQ ID NO:37, a nucleic acid sequence comprising SEQ ID NO:39, and a nucleic acid sequence of SEQ ID NO:41.

22. The method of claim 2, wherein the nucleic acid preparation comprises nucleic acid molecules comprising a nucleic acid sequence comprising SEQ ID NO:33, a nucleic acid sequence comprising SEQ ID NO:39, and a nucleic acid sequence of SEQ ID NO:41.

23. The method of claim 2, wherein the nucleic acid preparation comprises nucleic acid molecules comprising a nucleic acid sequence comprising SEQ ID NO:37, a nucleic acid sequence comprising SEQ ID NO:39, and a nucleic acid sequence of SEQ ID NO:41.

24. The method of claim 2, wherein the nucleic acid preparation comprises nucleic acid molecules comprising a nucleic acid sequence comprising SEQ ID NO:35, a nucleic acid sequence comprising SEQ ID NO:39, and a nucleic acid sequence of SEQ ID NO:41.

25. The method of claim 2, wherein the nucleic acid preparation comprises nucleic acid molecules comprising a nucleic acid sequence comprising SEQ ID NO:33, a nucleic acid sequence comprising SEQ ID NO:37, and a nucleic acid sequence comprising SEQ ID NO:39.

26. The method of claim 2, wherein the nucleic acid preparation comprises nucleic acid molecules comprising a nucleic acid sequence comprising SEQ ID NO:33, a nucleic acid sequence comprising SEQ ID NO:35, and a nucleic acid sequence comprising SEQ ID NO:39.

27. The method of claim 2, wherein the nucleic acid preparation comprises nucleic acid molecules comprising a nucleic acid sequence comprising SEQ ID NO:35, a nucleic acid sequence comprising SEQ ID NO:37, and a nucleic acid sequence comprising SEQ ID NO:39.

28. A cell population comprising induced hepatocytes obtained by the method of claim 1 or claim 2.

29. An isolated induced hepatocyte obtained by the method of claim 1 or claim 2.

30. A composition comprising a nucleic acid preparation, wherein the composition is capable of reprogramming a non-hepatocyte cell to an induced hepatocyte when the composition is introduced into a non-hepatocyte cell.

31. The composition of claim 30, wherein the non-hepatocyte cell is a human non-hepatocyte cell and the induced hepatocyte is a human induced hepatocyte.

32. The composition of claim 30, wherein the nucleic acid preparation comprises nucleic acid molecules encoding one or more reprogramming factors selected from the group consisting of CEBPA, GATA6, HHEX, HNF1B, HNF6A, FOXA1, FOXA2, FOXA3, GATA4, HNF1A, and HNF4A.

33. The composition of claim 30, wherein the nucleic acid preparation comprises nucleic acid molecules encoding one or more reprogramming factors selected from the group consisting of FOXA1, FOXA2, FOXA3, HNF1A, HNF4A, and GATA4.

34. The composition of claim 30, wherein the nucleic acid preparation comprises nucleic acid molecules encoding CEBPA, GATA6, HHEX, HNF1B, HNF6A, FOXA1, FOXA2, FOXA3, GATA4, HNF1A, and HNF4A.

35. The composition of claim 30, wherein the nucleic acid preparation comprises nucleic acid molecules encoding FOXA1, FOXA2, FOXA3, HNF1A, HNF4A, and GATA4.

36. The composition of claim 30, wherein the nucleic acid preparation comprises nucleic acid molecules encoding FOXA1, FOXA2, FOXA3, HNF1A, and HNF4A.

37. The composition of claim 30, wherein the nucleic acid preparation comprises nucleic acid molecules encoding FOXA1, FOXA3, HNF1A, and HNF4A.

38. The composition of claim 30, wherein the nucleic acid preparation comprises nucleic acid molecules encoding FOXA1, FOXA2, HNF1A, and HNF4A.

39. The composition of claim 30, wherein the nucleic acid preparation comprises nucleic acid molecules encoding FOXA2, FOXA3, HNF1A, and HNF4A.

40. The composition of claim 30, wherein the nucleic acid preparation comprises nucleic acid molecules encoding FOXA1, HNF1A, and HNF4A.

41. The composition of claim 30, wherein the nucleic acid preparation comprises nucleic acid molecules encoding FOXA3, HNF1A, and HNF4A.

42. The composition of claim 30, wherein the nucleic acid preparation comprises nucleic acid molecules encoding FOXA2, HNF1A, and HNF4A.

43. The composition of claim 30, wherein the nucleic acid preparation comprises nucleic acid molecules encoding FOXA1, FOXA3, and HNF1A.

44. The composition of claim 30, wherein the nucleic acid preparation comprises nucleic acid molecules encoding FOXA1, FOXA2, and HNF1A.

45. The composition of claim 30, wherein the nucleic acid preparation comprises nucleic acid molecules encoding FOXA2, FOXA3, and HNF1A.

46. The composition of claim 30, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, a nucleic acid sequence of SEQ ID NO:41, and a nucleic acid sequence comprising SEQ ID NO:43.

47. The composition of claim 30, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:37, a nucleic acid sequence comprising SEQ ID NO:39, and a nucleic acid sequence of SEQ ID NO:41.

48. The composition of claim 30, wherein the nucleic acid preparation comprises nucleic acid molecules comprising a nucleic acid sequence comprising SEQ ID NO:33, a nucleic acid sequence comprising SEQ ID NO:37, a nucleic acid sequence comprising SEQ ID NO:39, and a nucleic acid sequence of SEQ ID NO:41.

49. The composition of claim 30, wherein the nucleic acid preparation comprises nucleic acid molecules comprising a nucleic acid sequence comprising SEQ ID NO:33, a nucleic acid sequence comprising SEQ ID NO:35, a nucleic acid sequence comprising SEQ ID NO:39, and a nucleic acid sequence of SEQ ID NO:41.

50. The composition of claim 30, wherein the nucleic acid preparation comprises nucleic acid molecules comprising a nucleic acid sequence comprising SEQ ID NO:35, a nucleic acid sequence comprising SEQ ID NO:37, a nucleic acid sequence comprising SEQ ID NO:39, and a nucleic acid sequence of SEQ ID NO:41.

51. The composition of claim 30, wherein the nucleic acid preparation comprises nucleic acid molecules comprising a nucleic acid sequence comprising SEQ ID NO:33, a nucleic acid sequence comprising SEQ ID NO:39, and a nucleic acid sequence of SEQ ID NO:41.

52. The composition of claim 30, wherein the nucleic acid preparation comprises nucleic acid molecules comprising a nucleic acid sequence comprising SEQ ID NO:37, a nucleic acid sequence comprising SEQ ID NO:39, and a nucleic acid sequence of SEQ ID NO:41.

53. The composition of claim 30, wherein the nucleic acid preparation comprises nucleic acid molecules comprising a nucleic acid sequence comprising SEQ ID NO:35, a nucleic acid sequence comprising SEQ ID NO:39, and a nucleic acid sequence of SEQ ID NO:41.

54. The composition of claim 30, wherein the nucleic acid preparation comprises nucleic acid molecules comprising a nucleic acid sequence comprising SEQ ID NO:33, a nucleic acid sequence comprising SEQ ID NO:37, and a nucleic acid sequence comprising SEQ ID NO:39.

55. The composition of claim 30, wherein the nucleic acid preparation comprises nucleic acid molecules comprising a nucleic acid sequence comprising SEQ ID NO:33, a nucleic acid sequence comprising SEQ ID NO:35, and a nucleic acid sequence comprising SEQ ID NO:39.

56. The composition of claim 30, wherein the nucleic acid preparation comprises nucleic acid molecules comprising a nucleic acid sequence comprising SEQ ID NO:35, a nucleic acid sequence comprising SEQ ID NO:37, and a nucleic acid sequence comprising SEQ ID NO:39.

57. A method of screening a test compound, the method comprising contacting an induced hepatocyte obtained by the method of claim 1 or claim 2 with a test compound, and determining the effect of the test compound on the induced hepatocyte.


SEQUENCE LISTING (66 sequences)

SEQ ID NO: 1 - DNA, 23 bp, Artificial Sequence
  Description of Artificial Sequence Synthetic primer
ttaggaactgtgaagatggaagg

SEQ ID NO: 2 - DNA, 24 bp, Artificial Sequence
  Description of Artificial Sequence Synthetic primer
ctaggaagtgtttaggacgggtct

SEQ ID NO: 3 - DNA, 20 bp, Artificial Sequence
  Description of Artificial Sequence Synthetic primer
cactcggcttccagtatgct

SEQ ID NO: 4 - DNA, 22 bp, Artificial Sequence
  Description of Artificial Sequence Synthetic primer
ttaagaggagttcataatgggc

SEQ ID NO: 5 - DNA, 20 bp, Artificial Sequence
  Description of Artificial Sequence Synthetic primer
ctgggctcagtgaagatgga

SEQ ID NO: 6 - DNA, 23 bp, Artificial Sequence
  Description of Artificial Sequence Synthetic primer
ctaggatgcattaagcaaagagc

SEQ ID NO: 7 - DNA, 20 bp, Artificial Sequence
  Description of Artificial Sequence Synthetic primer
cgactctccaaaaccctcgt

SEQ ID NO: 8 - DNA, 23 bp, Artificial Sequence
  Description of Artificial Sequence Synthetic primer
ctagataacttcctgcttggtga

SEQ ID NO: 9 - DNA, 22 bp, Artificial Sequence
  Description of Artificial Sequence Synthetic primer
gtttctaaactgagccagctgc

SEQ ID NO: 10 - DNA, 19 bp, Artificial Sequence
  Description of Artificial Sequence Synthetic primer
ttactgggaggaagaggcc

SEQ ID NO: 11 - DNA, 19 bp, Artificial Sequence
  Description of Artificial Sequence Synthetic primer
tatcagagcttggccatgg

SEQ ID NO: 12 - DNA, 21 bp, Artificial Sequence
  Description of Artificial Sequence Synthetic primer
ttacgcagtgattatgtcccc

SEQ ID NO: 13 - DNA, 34 bp, Artificial Sequence
  Description of Artificial Sequence Synthetic primer
cctaagaagaagcgtaaggagagcgacgagagcg

SEQ ID NO: 14 - DNA, 20 bp, Artificial Sequence
  Description of Artificial Sequence Synthetic primer
ctattcttcaccggcatctg

SEQ ID NO: 15 - DNA, 48 bp, Artificial Sequence
  Description of Artificial Sequence Synthetic oligonucleotide
ccttccatcttcacagttcctaacatggtggctcttatatttcttctt

SEQ ID NO: 16 - DNA, 34 bp, Artificial Sequence
  Description of Artificial Sequence Synthetic oligonucleotide
cccgcagaaggcagcctaggaagtgtttaggacg

SEQ ID NO: 17 - DNA, 45 bp, Artificial Sequence
  Description of Artificial Sequence Synthetic oligonucleotide
agcatactggaagccgagtgcatggtggctcttatatttcttctt

SEQ ID NO: 18 - DNA, 37 bp, Artificial Sequence
  Description of Artificial Sequence Synthetic oligonucleotide
cccgcagaaggcagcttaagaggagttcataatgggc

SEQ ID NO: 19 - DNA, 44 bp, Artificial Sequence
  Description of Artificial Sequence Synthetic oligonucleotide
ccatcttcactgagcccagcatggtggctcttatatttcttctt

SEQ ID NO: 20 - DNA, 37 bp, Artificial Sequence
  Description of Artificial Sequence Synthetic oligonucleotide
cccgcagaaggcagcctaggatgcattaagcaaagag

SEQ ID NO: 21 - DNA, 44 bp, Artificial Sequence
  Description of Artificial Sequence Synthetic oligonucleotide
cgagggttttggagagtcgcatggtggctcttatatttcttctt

SEQ ID NO: 22 - DNA, 35 bp, Artificial Sequence
  Description of Artificial Sequence Synthetic oligonucleotide
cccgcagaaggcagcctagataacttcctgcttgg

SEQ ID NO: 23 - DNA, 47 bp, Artificial Sequence
  Description of Artificial Sequence Synthetic oligonucleotide
gcagctggctcagtttagaaaccatggtggctcttatatttcttctt

SEQ ID NO: 24 - DNA, 33 bp, Artificial Sequence
  Description of Artificial Sequence Synthetic oligonucleotide
cccgcagaaggcagcttactgggaggaagaggc

SEQ ID NO: 25 - DNA, 44 bp, Artificial Sequence
  Description of Artificial Sequence Synthetic oligonucleotide
ccatggccaagctctgatacatggtggctcttatatttcttctt

SEQ ID NO: 26 - DNA, 36 bp, Artificial Sequence
  Description of Artificial Sequence Synthetic oligonucleotide
cccgcagaaggcagcttacgcagtgattatgtcccc

SEQ ID NO: 27 - DNA, 48 bp, Artificial Sequence
  Description of Artificial Sequence Synthetic oligonucleotide
ctctccttacgcttcttcttaggcatggtggctcttatatttcttctt

SEQ ID NO: 28 - DNA, 35 bp, Artificial Sequence
  Description of Artificial Sequence Synthetic oligonucleotide
cccgcagaaggcagcctattcttcaccggcatctg

SEQ ID NO: 29 - DNA, 87 bp, Artificial Sequence
  Description of Artificial Sequence Synthetic oligonucleotide
ttggaccctcgtacagaagctaatacgactcactatagggaaataagagagaaaagaagagtaagaagaaatataagagccaccatg

SEQ ID NO: 30 - DNA, 119 bp, Artificial Sequence
  Description of Artificial Sequence Synthetic polynucleotide
gctgccttctgcggggcttgccttctggccatgcccttcttctctcccttgcacctgtacctcttggtctttgaataaagcctgagtaggaagtgagggtctagaactagtgtcgacgc

SEQ ID NO: 31 - DNA, 20 bp, Artificial Sequence
  Description of Artificial Sequence Synthetic primer
ttggaccctcgtacagaagc

SEQ ID NO: 32 - DNA, 144 bp, Artificial Sequence
  Description of Artificial Sequence Synthetic primer
ttttttttttttttttttttttttttttttttttttttttttttttttttttttttttttttttttttttttttttttttttttttttttttttttttttttttttttttttttttttttcttcctactcaggctttattcaaa

SEQ ID NO: 33 - DNA, 1419 bp, Homo sapiens
atgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtaaccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctag

SEQ ID NO: 34 - protein, 472 aa, Homo sapiens [sequence omitted]

SEQ ID NO: 35 - DNA, 1392 bp, Homo sapiens
atgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggctggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggggcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccagccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaa

SEQ ID NO: 36 - protein, 463 aa, Homo sapiens
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 37 - DNA, 954 bp, Homo sapiens
atggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcctag

SEQ ID NO: 38 - protein, 317 aa, Homo sapiens [sequence omitted]

SEQ ID NO: 39 - DNA, 1545 bp, Homo sapiens
atggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacgggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccggcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagtaa

SEQ ID NO: 40 - protein, 514 aa, Homo sapiens
MVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHALYSHKPEVAQYTHTGLLPQTMLITDTTNLSALASLTPTKQVFTSDTEASSESGLHTPASQATTLHVPSQDPAGIQHLQPAHRLSASPTVSSSSLVLYQSSDSSNGQSHLLPSNHSVIETFISTQMASSSQ

SEQ ID NO: 41 - DNA, 1425 bp, Homo sapiens
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 42 - protein, 474 aa, Homo sapiens
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 43 - DNA, 1329 bp, Homo sapiens
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggtctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 44 - protein, 442 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 45 - DNA, 19 bp, Artificial Sequence
  Description of Artificial Sequence Synthetic primer
gagtcggccgacttctacg

SEQ ID NO: 46 - DNA, 18 bp, Artificial Sequence
  Description of Artificial Sequence Synthetic primer
tcacgcgcagttgcccat

SEQ ID NO: 47 - DNA, 17 bp, Artificial Sequence
  Description of Artificial Sequence Synthetic primer
gccttgactgacggcgg

SEQ ID NO: 48 - DNA, 17 bp, Artificial Sequence
  Description of Artificial Sequence Synthetic primer
tcaggccagggccaggg

SEQ ID NO: 49 - DNA, 16 bp, Artificial Sequence
  Description of Artificial Sequence Synthetic primer
cagtacccgcaccccg

SEQ ID NO: 50 - DNA, 27 bp, Artificial Sequence
  Description of Artificial Sequence Synthetic primer
tcatccagcattaaaatagcttttatc

SEQ ID NO: 51 - DNA, 18 bp, Artificial Sequence
  Description of Artificial Sequence Synthetic primer
gtgtccaagctcacgtcg

SEQ ID NO: 52 - DNA, 21 bp, Artificial Sequence
  Description of Artificial Sequence Synthetic primer
ccaggcttgtagaggacactg

SEQ ID NO: 53 - DNA, 17 bp, Artificial Sequence
  Description of Artificial Sequence Synthetic primer
aacgcgcagctgaccat

SEQ ID NO: 54 - DNA, 21 bp, Artificial Sequence
  Description of Artificial Sequence Synthetic primer
tcatgctttggtacaagtgct

SEQ ID NO: 55 - DNA, 44 bp, Artificial Sequence
  Description of Artificial Sequence Synthetic oligonucleotide
cgtagaagtcggccgactccatggtggctcttatatttcttctt

SEQ ID NO: 56 - DNA, 33 bp, Artificial Sequence
  Description of Artificial Sequence Synthetic oligonucleotide
cccgcagaaggcagctcacgcgcagttgcccat

SEQ ID NO: 57 - DNA, 42 bp, Artificial Sequence
  Description of Artificial Sequence Synthetic oligonucleotide
ccgccgtcagtcaaggccatggtggctcttatatttcttctt

SEQ ID NO: 58 - DNA, 32 bp, Artificial Sequence
  Description of Artificial Sequence Synthetic oligonucleotide
cccgcagaaggcagctcaggccagggccaggg

SEQ ID NO: 59 - DNA, 44 bp, Artificial Sequence
  Description of Artificial Sequence Synthetic oligonucleotide
cgggccccacgcccatgaccatggtggctcttatatttcttctt

SEQ ID NO: 60 - DNA, 35 bp, Artificial Sequence
  Description of Artificial Sequence Synthetic oligonucleotide
cccgcagaaggcagctcatccagcattaaaatagc

SEQ ID NO: 61 - DNA, 43 bp, Artificial Sequence
  Description of Artificial Sequence Synthetic oligonucleotide
cgacgtgagcttggacaccatggtggctcttatatttcttctt

SEQ ID NO: 62 - DNA, 35 bp, Artificial Sequence
  Description of Artificial Sequence Synthetic oligonucleotide
cccgcagaaggcagcccaggcttgtagaggacact

SEQ ID NO: 63 - DNA, 42 bp, Artificial Sequence
  Description of Artificial Sequence Synthetic oligonucleotide
atggtcagctgcgcgttcatggtggctcttatatttcttctt

SEQ ID NO: 64 - DNA, 35 bp, Artificial Sequence
  Description of Artificial Sequence Synthetic oligonucleotide
cccgcagaaggcagctcatgctttggtacaagtgc

SEQ ID NO: 65 - DNA, 120 bp, Artificial Sequence
  Description of Artificial Sequence Synthetic polynucleotide
tttttttttttttttttttttttttttttttttttttttttttttttttttttttttttttttttttttttttttttttttttttttttttttttttttttttttttttttttttttttt

SEQ ID NO: 66 - DNA, 120 bp, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa